SEQUENCE LISTING (26 sequences)

SEQ ID NO: 1 - protein, 229 aa, synthetic construct
MSQPRPLLSPPETEEQLLAQAQQLSGYTLGELAALVGLVTPENLKRDKGWIGVLLEIWLGASAGSKPEQDFAALGVELKTIPVDSLGRPLETTFVCVAPLTGNSGVTWETSHVRHKLKRVLWIPVEGERSIPLAQRRVGSPLLWSPNEEEDRQLREDWEELMDMIVLGQVERITARHGEYLQIRPKAANAKALTEAIGARGERILTLPRGFYLKKNFTSALLARHFLIQ

SEQ ID NO: 2 - protein, 229 aa, synthetic construct
MSQPRPLLSPPETEEQLLAQAQQLSGYTLGELAALVGLVTPENLKRDKGWIGVLLEIWLGASAGSKPEQDFAALGVELKTIPVDSLGRPLATTAVCVAPLTGNSGVTWETSHVRHKLKRVLWIPVEGERSIPLAQRRVGSPLLWSPNEEEDRQLREDWEELMDMIVLGQVERITARHGEYLQIRPKAANAKALTEAIGARGERILTLPRGFYLKKNFTSALLARHFLIQ

SEQ ID NO: 3 - protein, 223 aa, synthetic construct
RQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 4 - protein, 434 aa, synthetic construct
FKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSSGGGGSGGGGSGGGGSGGSGGGSSGGGGSGGGGSGGGGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 5 - protein, 330 aa, synthetic construct
CRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKEDGNALLVINDKIIDKINEIKNILQDIPDEYKLLNEKISEQFNSEEVSDELLRDLVTHLPTKESEPANFKLARKYLQEIMEIVGDELSKMWEDERYVRQTFADLD

SEQ ID NO: 6 - protein, 330 aa, synthetic construct
CRSHADRGRWEKNLRSYTTDRDAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNEKISEQFNSEEVSDELLRDLVTHLPTKESEPANFKLARKYLQEIMEIVGDELSKMWEDERYVRQTFADLD

SEQ ID NO: 7 - protein, 315 aa, synthetic construct
KDPGRHDDNMRLYNHDRRAFMWWSEGDWALADALYNKAGAGKCADPDCQKEVEKISPDHVGPISCGFKQIPFFKPLCASCNSAKNRRFSYQDVKELLKYENYTGDSVASWQVRALWDNCKHLVKNDDDSKLLSNLMRSLQDYYLRSLYKLFSNGFAHLLSYFLTPEYAHYKITFEGLNTSTLEYERYYKTFKKTKSTSSLAARIVRIAFEELEIYNSKDINEDKLIKFDTSSWEKDFENIISYATKNLSLDEEASKWNKVLTDKNLSSTEKDKKISSLLEDKNYEVYKKQFYILKDLLVEHFNKIGEQIAKDYMK

SEQ ID NO: 8 - protein, 266 aa, synthetic construct
CRASQDKGRSKENLKSYTKDRDAYEYWSDGNIHAANQFMGSPFFNNISADHIGPISLGFVHDPRYLQPMSGGDNSSKRDRLQLDDIEKIIETEKRTNVYPMSWYSKLIWEYIKKNYSTHKSLISGVYRDALKQNMSNFMYILWYILEHCNQDGEHFLEEALLKPNYDYFQYSYTFNELGEIVSINPRHFTDRNQYETERYKRIAFESVYDYNEKENRNIKANLIDNEQRMLNKLCQEISSGVPVEQCKKLLIELMEVIQKRIISTL

SEQ ID NO: 9 - protein, 227 aa, synthetic construct
IPEELFNWPRTDKVNFKSPQGLIKYDELCYQLEKAVGSKKAYCLSNNAGAKPQKLESLKEWINSQKKLFDKAPKLTPPAEFNMKLDAFPVTSNNNYYVTTSKNILYLFDYWKDLRIAIETAFPRLKGKLPTDIDEKPALIYICNSVKPGRLFGDPFTGQLSAFSTIFGKKNIDMPRIVVAYYPHQIYSQALPKNNKSNKGITLKKELTDFLIFHGGVVVKLNEGKAY

SEQ ID NO: 10 - protein, 348 aa, synthetic construct
MYIYMSTPQAKTKYYEQRFVNDFYKELERNKVSLPVTIVLKDNLGIKQVIQNGSGVRVLRDKANAKSPSKIKSEELGRHVTSKADIALFTEEKNGTKVDVAWISPQSHKDFLGKKITPAQYFDASSDVMFKTKIGQPKEIKELKNKMISLSVPLTATKYCWPKYKSGTSLRIWDDVQSTILMNMAIFGVEFGKAYCRNNANILMVGDPLIEVKDDKTIILTTKENGFSLANGFAEYIPSKDKPIFFTKPTSGKKTVVDGKTIEGVSVWIIYRSYAGSKNRKIDDVLKNKIELISSSCSVKKKDNFVSIMQSKKITSPPKSKKITSPPKSKKITSPSKSKKITNFFMKK

SEQ ID NO: 11 - protein, 197 aa, synthetic construct
NNSFNPVRTKDQLHESAVITREKKILLKEPEILQKIKNRNNGEGLKSIIWKKFDKKCFNCEKELTIEEVRLDHTRPLAYLWPIDEHATCLCEKCNNTKHDMFPIDFYQGDEDKLRRLARITGLDYESLVKRDVNEVELARIINNIEDFATNVEARTFRSIRNKVKEVRPDTDLFEILKSKNINLYNELQYELLTRKD

SEQ ID NO: 12 - protein, 249 aa, synthetic construct
YHLEELLFENNEKKFAENQKNEWDEILAYMDLLISPKPISIEIADKEISIPSGERPAYFEWVLWRAFLALNHLIIEPQQCRRFKVDQDFKPIHNAPGGGADVIFEYENFKILGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 13 - protein, 488 aa, synthetic construct
MTEYDLHLYADSFHEGHWCCENLAKIAQSDGGKHQIDYLQGFIPRHSLIFSDLIINITVFGSYKSWKHLPKQIKDLLFWGKPDFIAYDPKNDKILFAVEETGAVPTGNQALQRCERIYGSARKQIPFWYLLSEFGQHKDGGTRRDSIWPTIMGLKLTQLVKTPSIILHYSDINNPEDYNSGNGLKFLFKSLLQIIINYCTLKNPLKGMLELLSIQYENMLEFIKSQWKEQIDFLPGEEILNTKTKELARMYASLAIGQTVKIPEELFNWPRTDKVNFKSPQGLIKYDELCYQLEKAVGSKKAYCLSNNAGAKPQKLESLKEWINSQKKLFDKAPKLTPPAEFNMKLDAFPVTSNNNYYVTTSKNILYLFDYWKDLRIAIETAFPRLKGKLPTDIDEKPALIYICNSVKPGRLFGDPFTGQLSAFSTIFGKKNIDMPRIVVAYYPHQIYSQALPKNNKSNKGITLKKELTDFLIFHGGVVVKLNEGKAY

SEQ ID NO: 14 - protein, 328 aa, synthetic construct
MQIEQLMKSLTIYFDDIQEGLWFKNLHPLLESASLEAITGSLKRNPNLADVLKYDRPDIILTLNQTPILVIERTIEVPSGHNVGQRYGRLAAASEAGVPLVYFGPYAARKHGGATEGPRYMNLRLFYALDVMQKVNGSAITTINWPVDQNFEILQDPSKDKRMKEYLEMFFDNLLKYGIAGINLAIRNSSFQAEQLAEREKFVETMITNPEQYDVPPDSVQILNAERFFNELGISENKRIICDEVVLYQVGMTYVRSDPYTGMALLYKYLYILGSERNRCLILKFPNITTDMWKKVAFGSRERKDVRIYRSVSDGILFADGYLSKEEL

SEQ ID NO: 15 - protein, 188 aa, synthetic construct
KSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSQGPSAD

SEQ ID NO: 16 - protein, 186 aa, synthetic construct
MQDILDFYEEVEKTINPPNYFEWNTYRVFKKLGSYKNLVPNFKLDDSGHPIGNAIPGVEDILVEYEHFSILIECSLTIGEKQLDYEGDSVVRHLQEYKKKGIEAYTLFLGKSIDLSFARHIGFNKESEPVIPLTVDQFKKLVTQLKGDGEHFNPNKLKEILIKLLRSDLGYDQAEEWLTFIEYNLK

SEQ ID NO: 17 - protein, 217 aa, synthetic construct
MTDYRYSFELSEEIARWAFEIKTKNTDWFVAFSNPTAGPWKRVMAIDKASNREGEVHRFGREDERPDIILVNDNISLILILEAKEKLNQLISKSQVDKSVDVFLTLSSILKEKSDNNYWGDRTKYINVLGILWGSEQETSQKDIDNAFRVYRDSLVKNLKEINPTPTNICTDILVGVESIKNKKEEISIKIHVSNIYAEIYPKFTGKHLLEKLAVLN

SEQ ID NO: 18 - protein, 164 aa, synthetic construct
MKITEGIVHVAMRHFLKSNGWKLIAGQYPGGSDDELTALNIVDPVVARDNSPDPRRHSLGKIVPDLIAYKNDDLLVIEAKPKYSQDDRDKLLYLLSERKHDFYAALEKFATERNHPELLPVSKLNIIPGLAFSASENKFKKDPGFVYIRVSGIFEAFMEGYDWG

SEQ ID NO: 19 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 20 - protein, 409 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 21 - protein, 397 aa, synthetic construct
MSQPRPLLSPPETEEQLLAQAQQLSGYTLGELAALVGLVTPENLKRDKGWIGVLLEIWLGASAGSKPEQDFAALGVELKTIPVDSLGRPLETTFVCVAPLTGNSGVTWETSHVRHKLKRVLWIPVEGERSIPLAQRRVGSPLLWSPNEEEDRQLREDWEELMDMIVLGQVERITARHGEYLQIRPKAANAKALTEAIGARGERILTLPRGFYLKKNFTSALLARHFLIQGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 22 - protein, 397 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINGSMSQPRPLLSPPETEEQLLAQAQQLSGYTLGELAALVGLVTPENLKRDKGWIGVLLEIWLGASAGSKPEQDFAALGVELKTIPVDSLGRPLETTFVCVAPLTGNSGVTWETSHVRHKLKRVLWIPVEGERSIPLAQRRVGSPLLWSPNEEEDRQLREDWEELMDMIVLGQVERITARHGEYLQIRPKAANAKALTEAIGARGERILTLPRGFYLKKNFTSALLARHFLIQ

SEQ ID NO: 23 - protein, 391 aa, synthetic construct
RQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRFGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 24 - protein, 391 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINGSRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 25 - protein, 22 aa, synthetic construct
AEAAAKEAAAKEAAAKEAAAKA

SEQ ID NO: 26 - protein, 32 aa, synthetic construct
GSGSSGGSSGSETPGTSESATPESSGGSSGGS

(19) United States
(12) Patent Application Publication
(10) Pub. No.: US 2026/0207779 A1
(43) Pub. Date: Jul. 23, 2026

(54) NUCLEOBASE EDITOR SYSTEMS AND METHODS OF USE THEREOF

(71) Applicant: Peking University, Beijing (CN)

(72) Inventors: Wensheng WEI, Beijing (CN); Zongyi YI, Beijing (CN); Xiaoxue ZHANG, Beijing (CN)

(21) Appl. No.: 19/144,170

(22) PCT Filed: Dec. 25, 2023
(86) PCT No.: PCT/CN2023/141468

§ 371 Date: Jun. 27, 2025

(30) Foreign Application Priority Data
Dec. 30, 2022 (WO) PCT/CN2022/144031
Apr. 13, 2023 (WO) PCT/CN2023/088117

(51) Int. Cl. A61K 48/00; C12N 9/16; C12N 9/78; C12N 15/11

(52) U.S. Cl. CPC A61K 48/005; C12N 9/16; C12N 9/78; C12N 15/11; C07K 2319/07; C07K 2319/81; C12Y 301/21004; C12Y 305/04002; C12Y 305/04005

(57) ABSTRACT

Provided are systems for strand-specific editing of DNA, including mitochondrial DNA in humans. The systems provided herein comprise a single stand nickase and a deaminase each, or together, associated with a double-stranded DNA binding polypeptide such that DNA editing occurs in an editing region. Also provided are components of the systems for editing DNA, and methods of use thereof.

### CROSS-REFERENCE TO RELATED APPLICATIONS

This patent application claims priority to and benefit of International Application Nos. PCT/CN2022/144031, filed on Dec. 30, 2022, and PCT/CN2023/088117, filed on Apr. 13, 2023, the contents of each of which are incorporated herein by reference in their entirety.

### TECHNICAL FIELD

The present application is directed to systems for strand-specific editing of DNA, including mitochondrial DNA in humans. The systems provided herein comprise a single-stranded nickase and a deaminase each, or together, associated with a double-stranded DNA binding polypeptide such that DNA editing occurs in an editing region. Also provided herein are components of the systems for editing DNA taught herein, and methods of use thereof.

### BACKGROUND

Mitochondrial DNA (mtDNA) exists in multiple copies and is heteroplasmic in the majority of human cells with mitochondrial disease. mtDNA mutations have been linked to many human diseases, approximately 95% of which are point mutations. In certain mitochondrial diseases, wild-type mtDNA coexists with mutant mtDNA, and the ratio of wild-type to mutant mtDNA often correlates with the severity of the clinical phenotype. Theoretically, treatments for such mtDNA-associated disease could involve mtDNA editing systems. The CRISPR system has been widely used in nuclear genome base editing. However, it is still not feasible to apply such a system to edit the mitochondrial genome because of the lack of an effective way to deliver guide RNA into this organelle. Thus, robust technologies for mtDNA base editing are therefore highly desirable to help reveal the underlying mechanisms of pathogenesis and identify ways to correct disease-causing mutations for a cure.

### BRIEF SUMMARY

In some aspects, provided herein is a nucleobase editor system comprising: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase such that a site action for the ss-nickase and a site of action for the deaminase are within an editing region on the dsDNA.

In some embodiments, the ss-nickase of the first unit recognizes a recognition sequence present in the editing region of the dsDNA. In some embodiments, the recognition sequence of the ss-nickase is a palindromic recognition sequence. In some embodiments, the recognition sequence of the ss-nickase is 5′-GATC-3′. In some embodiments, the recognition sequence of the ss-nickase is a non-palindromic recognition sequence. In some embodiments, the recognition sequence of the ss-nickase is 5′-GATD-3′, where D is a base selected from the group consisting of G, A, and T. In some embodiments, the recognition sequence of the ss-nickase is 5′-GAGTC-3′. In some embodiments, the recognition sequence of the ss-nickase is hemimethylated.

In some embodiments, the first dsDNA binding polypeptide of the first unit comprises a transcription activator-like effector (TALE) domain. In some embodiments, the first dsDNA binding polypeptide of the first unit comprises a zinc finger (ZF) domain. In some embodiments, the first dsDNA binding polypeptide of the first unit specifically associates with a portion of the dsDNA upstream of the editing region on the dsDNA sequence. In some embodiments, the first dsDNA binding polypeptide of the first unit specifically associates with a portion of the dsDNA downstream of the editing region on the dsDNA sequence.

In some embodiments, the site of action of the ss-nickase is on the same strand of the dsDNA that is bound by the first dsDNA binding polypeptide of the first unit. In some embodiments, the first dsDNA binding polypeptide of the first unit binds the dsDNA 5-9 bases away from the recognition sequence of the ss-nickase. In some embodiments, the site of action of the ss-nickase is on the opposite strand of the dsDNA that is bound by the first dsDNA binding polypeptide of the first unit. In some embodiments, the first dsDNA binding polypeptide of the first unit binds 0-4 bases away from the recognition sequence of the ss-nickase.

In some embodiments, the ss-nickase is heterologous. In some embodiments, the ss-nickase of the first unit is a type I nickase, type II nickase, type III nickase, or a type IV nickase. In some embodiments, the ss-nickase is MutH or Nt.BspD6I, or a nickase derived therefrom.

In some embodiments, the second dsDNA binding polypeptide of the second unit comprises a transcription activator-like effector (TALE) domain. In some embodiments, the second dsDNA binding polypeptide of the second unit comprises a zinc finger (ZF) domain. In some embodiments, the second dsDNA binding polypeptide binds to the stand of the dsDNA not bound by the first dsDNA binding polypeptide of the first unit. In some embodiments, the second dsDNA binding polypeptide of the second unit specifically associates with a portion of the dsDNA downstream of the editing region on the dsDNA sequence. In some embodiments, the second dsDNA binding polypeptide of the second unit specifically associates with a portion of the dsDNA upstream of the editing region on the dsDNA sequence.

In some embodiments, the site of action of the deaminase is part of the non-nicked strand of the dsDNA. In some embodiments, the deaminase is heterologous. In some embodiments, the deaminase is a single-stranded deaminase (ss-deaminase). In some embodiments, the deaminase of the second unit is a cytosine-to-uracil deaminase, a 5-methylcytosine-to-thymine deaminase, a guanine-to-xanthine deaminase, an adenine-to-hypoxanthine deaminase, or an adenine-to-inosine deaminase. In some embodiments, the deaminase is TadA8e, APOBEC, or AID.

In some embodiments, the editing region on the dsDNA is 1-24 base pairs in length. In some embodiments, the site of action of the ss-nickase is no more than 10 base pairs from the site of action of the deaminase.

In some embodiments, the first unit is a fusion polypeptide, wherein the first dsDNA binding polypeptide of the first unit is fused to the ss-nickase of the first unit. In some embodiments, the first dsDNA binding polypeptide of the first unit is fused to the C-terminus of the ss-nickase of the first unit. In some embodiments, the first dsDNA binding polypeptide of the first unit is fused to the N-terminus of the ss-nickase of the first unit. In some embodiments, the first unit further comprises a linker associating the first dsDNA binding polypeptide and the ss-nickase. In some embodiments, the second unit is a fusion polypeptide, wherein the second dsDNA binding polypeptide of the second unit is fused to the deaminase of the second unit. In some embodiments, the second dsDNA binding polypeptide of the second unit is fused to the C-terminus of the deaminase of the second unit. In some embodiments, the second dsDNA binding polypeptide of the second unit is fused to the N-terminus of the deaminase of the second unit. In some embodiments, the second unit further comprises a linker associating the second dsDNA binding polypeptide and the deaminase. In some embodiments, the linker of the first unit and/or the linker of the second unit comprise a polypeptide linker. In some embodiments, the polypeptide linker is from 2-100 amino acids in length.

In some embodiments, the first dsDNA binding polypeptide and the ss-nickase of the first unit associate non-covalently. In some embodiments, the second dsDNA binding polypeptide and the deaminase of the second unit associate non-covalently.

In some embodiments, the first unit further comprises a mitochondrial localization signal (MLS). In some embodiments, the MLS is positioned at the N-terminus of the first unit. In some embodiments, the second unit further comprises a mitochondrial localization signal (MLS). In some embodiments, the MLS is positioned at the N-terminus of the second unit.

In some embodiments, the dsDNA is a circularized dsDNA. In some embodiments, the dsDNA is mitochondrial DNA (mtDNA). In some embodiments, the dsDNA is a B-DNA conformation.

In other aspects, provided herein is a nucleobase editor system comprising: a single-stranded (ss-) nickase; a deaminase; and a double-stranded (ds) DNA binding polypeptide, wherein the ss-nickase, the deaminase, and dsDNA binding polypeptide form a complex configured such that the dsDNA binding polypeptide, when associated with a dsDNA, positions the ss-nickase and the deaminase such that a site action for the ss-nickase and a site of action for the deaminase are within an editing region on the dsDNA.

In other aspects provided herein is a non-naturally occurring polynucleotide encoding: a first unit comprising a first double-stranded (ds) DNA binding polypeptide associated with a single-stranded (ss-) nickase; and/or a second unit comprising a second dsDNA binding polypeptide associated with a deaminase.

In other aspects provided herein is a non-naturally occurring polynucleotide encoding: a first unit comprising a first double-stranded (ds) DNA binding polypeptide associated with a single-stranded (ss-) nickase; and a second unit comprising a second dsDNA binding polypeptide associated with a deaminase, wherein the first unit and second unit, when expressed, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In other aspects, provided herein is a non-naturally occurring polynucleotide encoding: a single-stranded (ss-) nickase; a deaminase; and a double-stranded (ds) DNA binding polypeptide, wherein the ss-nickase, the deaminase, and dsDNA binding polypeptide, when expressed, form a complex configured such that the dsDNA binding polypeptide, when associated with a dsDNA, positions the ss-nickase and the deaminase with a site action for the ss-nickase and a site of action for the deaminase within an editing region on the dsDNA.

In other aspects, provided herein is a method of editing a target nucleotide in an editing region in a cell, the method comprising delivering any nucleobase editor system described herein or the polynucleotide of any nucleobase editor system described herein to the cell. In some embodiments, the editing region is on a mitochondrial DNA.

In other aspects, provided herein is a method of treating an individual having a disease associated with a DNA mutation, the method comprising administering one or more polynucleotides encoding: a first unit comprising a first double-stranded (ds) DNA binding polypeptide associated with a single-stranded (ss-) nickase; and a second unit comprising a second dsDNA binding polypeptide associated with a deaminase, wherein the first unit and second unit, when expressed in the individual, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA. In some embodiments, the mutated DNA is mitochondrial DNA.

In other aspects, provided herein is a kit for a nucleobase editing system, the kit comprising: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In other aspects, provided herein is a kit for a nucleobase editor system, the kit comprising one or more polynucleotides encoding: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system, when expressed, is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In other aspects, provided herein is a non-naturally occurring polypeptide having nickase activity, the non-naturally occurring polypeptide comprising the amino acid sequence of SEQ ID NO:1 with the following mutations of E91A and F94A (SEQ ID NO:2). In some embodiments, the non-naturally occurring polypeptide is isolated.

In other aspects, provided herein is a polynucleotide encoding a non-naturally occurring polypeptide of SEQ ID NO:2.

All references cited herein, including patent applications and publications, are incorporated by reference in their entirety.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIG. 1A shows the mitochondrial A-to-G editing efficiency of HEK293T cells treated with paired TALE-TadA8e(V106W) at the MT-RNR2 site. FIG. 1B shows the mitochondrial A-to-G editing efficiency of HEK293T cells treated with paired TALE-TadA8e(V106W) at the MT-ND1 site. FIG. 1C shows the mitochondrial A-to-G editing efficiency of HEK293T cells treated with paired TALE-TadA8e(V106W) at the MT-ND4 site. FIG. 1D shows the mitochondrial A-to-G editing efficiency of HEK293T cells treated with Left TALE-MutH and Right TALE-TadA8e(V106W) at MT-RNR2 site. FIG. 1E shows the mitochondrial A-to-G editing efficiency of HEK293T cells treated with Left TALE-MutH and Right TALE-TadA8e(V106W) at MT-ND) site. FIG. 1F shows the mitochondrial A-to-G editing efficiency of HEK293T cells treated with Left TALE-MutH and Right TALE-TadA8e(V106W) at MT-ND4 site. FIG. 1G shows the mitochondrial A-to-G editing efficiency of HEK293T cells treated with Left TALE-MutH(D70A) and Right TALE-TadA8e(V106W) at MT-RNR2. FIG. 1H shows the mitochondrial A-to-G editing efficiency of HEK293T cells treated with Left TALE-MutH(D70A) and Right TALE-TadA8e(V106W) at MT-ND1. FIG. 1I shows the mitochondrial A-to-G editing efficiency of HEK293T cells treated with Left TALE-MutH(D70A) and Right TALE-TadA8e(V106W) at MT-ND4. FIG. 1J shows product distributions at MT-RNR2 site from mitochondrial A-to-G editing of HEK293T cells treated with Left TALE-MutH and Right TALE-TadA8e(V106W). FIG. 1K shows product distributions at MT-ND1 site from mitochondrial A-to-G editing of HEK293T cells treated with Left TALE-MutH and Right TALE-TadA8e(V106W). FIG. 1L shows product distributions at MT-ND4 site from mitochondrial A-to-G editing of HEK293T cells treated with Left TALE-MutH and Right TALE-TadA8e(V106W). FIG. 1M shows a time course analysis of editing efficiencies at the MT-RNR2 site from mitochondrial A-to-G editing of HEK293T cells treated with Left TALE-MutH and Right TALE-TadA8e(V106W). FIG. 1N shows a time course analysis of editing efficiencies at the MT-ND) site from mitochondrial A-to-G editing of HEK293T cells treated with Left TALE-MutH and Right TALE-TadA8e(V106W). FIG. 1O shows a time course analysis of editing efficiencies at the MT-ND4 site from mitochondrial A-to-G editing of HEK293T cells treated with Left TALE-MutH and Right TALE-TadA8e(V106W). FIG. 1P shows a predictive model for improving the editing efficiency of mitoDNA by combining a nickase with TadA8e(V106W). TALE-nickase binds the target DNA and nicks the dsDNA. The nicked dsDNA is prone to form single-stranded DNA structures. TALE-TadA8e(V106W) binds the target DNA and efficiently deaminates the adenine(s) on the resulting ssDNA. The resulting inosine(s) can be permanently converted to guanine(s) following DNA repair or DNA replication.

FIG. 2A shows different orientations of mitoABE^{MutH} when MutH is moved away from 3 bp to 5′-GATC-3′ and the adenines on different strands are edited at MT-RNR2 site 1, MT-ND) site 1 and MT-ND4 site 1. Up, Left TALE-fused MutH and Right TALE-fused TadA8e(V106W). Down, Left TALE-fused TadA8e(V106W) and Right TALE-fused MutH. Values and errors reflect the mean±s.d. of n=3 independent biological replicates. FIG. 2B shows different orientations of mitoABE^{MutH} when the 5 bp distance from 5′-GATC-3′ and the adenines on different strands are edited at MT-RNR2 site 2 and MT-ND4 site 1. Top, Left TALE-fused TadA8e(V106W) and Right TALE-fused MutH. Down, Left TALE-fused MutH and Right TALE-fused TadA8e(V106W). Values and errors reflect the mean±s.d. of n=3 independent biological replicates. FIG. 2C shows the editing efficiency of mitoABE^{MutH*} with MutH and TadA8e(V106W) at different distances from 5′-GATC-3′ at MT-ND4. Values and errors reflect the mean±s.d. of n=3 independent biological replicates. FIG. 2D shows the editing efficiency of mitoABE^{MutH} with MutH and TadA8e(V106W) at different distances from 5′-GATC-3′ at MT-RNR2 site 1. FIG. 2E shows the editing efficiency of mitoABE^{MutH} with MutH and TadA8e(V106W) at different distances from 5′-GATC-3′ at MT-ND4. FIG. 2F shows the strand-specific nicking of MutH at different distances from 5′-GATC-3′. When the distance between TALE-MutH and 5′-GATC-3′ is 0-4 bp, the opposite strand is nicked, thus causing the editing of the TALE-MutH recognition strand; when the distance between TALE-MutH and 5′-GATC-3′ is 5-9 bp, the TALE-MutH recognition strand is nicked, thus causing the editing of the opposite strand. FIG. 2G shows editing efficiency of a Left-TALE-MutH, right-TALE-TadA8E(V106W) system containing a linker sequence between the TALE and MutH at the MT-ND1 site. FIG. 2H shows editing efficiency of a Left-TALE-MutH, right-TALE-TadA8E(V106W) system containing a linker sequence between the TALE and MutH at the MT-ND4 site. FIG. 2G shows editing efficiency of a Left-TALE-TadA8E(V106W), right-TALE-MutH system containing a linker sequence between the TALE and MutH at the MT-ND1 site. FIG. 2H shows editing efficiency of a Left-TALE-TadA8E(V106W), right-TALE-MutH system containing a linker sequence between the TALE and MutH at the MT-ND4 site.

FIG. 3A shows the crystal structure of key amino acids of MutH interacting with hemimethylated 5′-GATC-3′. FIG. 3B shows the editing efficiency of MutH mutants (including K48A, E91A, F94A, R184A, Y212S and double mutation of E91A and F94A) combined with TadA8e(V106W) at the 5′-GATC-3′ position. FIG. 3C shows the editing efficiencies of target spacing regions at the 5′-GATA-3′ with different mitoABE^{MutH*} orientations and distances. FIG. 3D shows the editing efficiencies of target spacing regions at the 5′-GATG-3′ with different mitoABE^{MutH*} orientations and distances. FIG. 3E shows the editing efficiencies of target spacing regions at the 5′-GATT-3′ positions with different mitoABE^{MutH*} orientations and distances. FIG. 3F shows the editing efficiencies of target spacing regions at the 5′-GATA-3′ with different mitoABE^{MutH} orientations and distances to 5′-GATD-3′. All data points from n=3 biologically independent experiments are shown. FIG. 3G shows the editing efficiencies of target spacing regions at the 5′-GATG-3′ with different mitoABE^{MutH} orientations and distances to 5′-GATD-3′. All data points from n=3 biologically independent experiments are shown. FIG. 3H shows the editing efficiencies of target spacing regions at the 5′-GATT-3′ positions with different mitoABE^{MutH} orientations and distances to 5′-GATD-3′. All data points from n=3 biologically independent experiments are shown. FIG. 3I shows the designable targeting range of TALE-MutH in human mitochondria. FIG. 3J shows the designable targeting range of TALE-MutH* in human mitochondria. The numbers 0, 2, 4, and 6 indicate the frequency of MutH and MutH* recognition sequences within a 40 bp region.

FIG. 4A shows the predicted structure of the screened nickases, BsaI, BsmBI, BsmAI, Nb. BsrDI, Nt. CviPII, BspQI. The arrow indicates the segmentation location, and the C-terminus of the protein is the cleavage domain of the corresponding protein that was selected. Full-length Nt.CviPII was used. FIG. 4B shows the results of a mitochondrial base editing screen of nickases without sequence restriction. All data points from n=3 biologically independent experiments are shown. FIG. 4C shows the editing efficiencies of different mitochondrial sites when Left TALE-Nt.BspD6I(C) was combined with Right TALE-TadA8e(V106W). All data points from n=3 biologically independent experiments are shown. FIG. 4D shows the editing efficiencies of different mitochondrial sites when Right TALE-Nt.BspD6I(C) combined with Left TALE-TadA8e(V106W). All data points from n=3 biologically independent experiments are shown. FIG. 4E shows the editing efficiency of a Left-TALE-Nt.BspD6I(C), Right-TALE-TadA8e(V100W) system containing a linker sequence between the TALE and Nt.BspD6I(C) at the MT-ND5 site 2. FIG. 4F shows the editing efficiency of a Left-TALE-Nt.BspD6I(C), Right-TALE-TadA8e(V100W) system containing a linker sequence between the TALE and Nt.BspD6I(C) at the MT-ND4 site. FIG. 4G shows the editing efficiency of a Left-TALE-TadA8e(V100W), Right-TALE-Nt.BspD6I(C) system containing a linker sequence between the TALE and Nt.BspD6I(C) at the MT-ND1 site. FIG. 4H shows the editing efficiency of a Left-TALE-TadA8e(V100W), Right-TALE-Nt.BspD6I(C) system containing a linker sequence between the TALE and Nt.BspD6I(C) at the MT-ND4 site.

FIG. 5A shows the editing efficiency of mitoCBE at MT-ND4. FIG. 5B shows the editing efficiency of mitoCBE at MT-RNR2 site 3. FIG. 5C shows the editing efficiency of mitoCBE at MT-RNR2 site 1. FIGS. 5D-5F compare the editing profiles of mitoCBEs and DdCBEs at MT-ND4 (FIG. 5D), MT-RNR2 site 3 (FIG. 5E), and MT-RNR2 site 1 (FIG. 5F). For each position, the editing percentage is shown for DdCBE (Left-G1397-N), DdCBE (Left-G1397-C), and mitoCBE (Right-MutH) as three consecutive bars in that order.

FIG. 6A shows the editing efficiency of monomeric mitoABEs, including TALE-MutH-TadA8e(V106W), TALE-TadA8e(V106W)-MutH, and TALE-Nt.BspD6I-TadA8e(V106W), TALE-TadA8e(V106W)-Nt.BspD6I at MT-ND1. FIG. 6B shows the editing efficiency of monomeric mitoABEs, including TALE-MutH-TadA8e(V106W), TALE-TadA8e(V106W)-MutH, and TALE-Nt.BspD6I-TadA8e(V106W), TALE-TadA8e(V106W)-Nt.BspD6I at MT-ND4. In FIG. 6A and FIG. 6B, the box represents the editing window of the dimeric mitoABEs. FIG. 6C shows the editing efficiency of monomeric mitoCBEs, including TALE-MutH-rAPOBEC1-UGI, TALE-rAPOBEC1-UGI-MutH, TALE-Nt.BspD6I(C)-rAPOBEC1-UGI, and TALE-rAPOBEC1-UGI-Nt.BspD6I(C) at MT-ND1. FIG. 6D shows the editing efficiency of monomeric mitoCBEs, including TALE-MutH-rAPOBEC1-UGI, TALE-rAPOBEC1-UGI-MutH, TALE-Nt.BspD6I(C)-rAPOBEC1-UGI, and TALE-rAPOBEC1-UGI-Nt.BspD6I(C) at MT-ND4. All data points form n=3 biologically independent experiments.

FIGS. 7A-7K show the average frequency and mitochondrial genome position of each unique single nucleotide variant (SNV) for untreated HEK293T cells (FIG. 7A) and HEK293T cells treated with nontargeting mitoABE^{MutH} (FIG. 7B), nontargeting mitoABE^{Nt.BspD6I(C)} (FIG. 7C), MT-ND4-targeting mitoABE^{MutH} (Left-TALE-MutH with Right-TALE-TadA8e(V106W)) (FIG. 7D), MT-ND4-targeting mitoABE^{MutH} (Left-TALE-TadA8e(V106W) with Right-TALE-MutH) (FIG. 7E), MT-RNR2-targeting mitoABE^{MutH} (Left-TALE-MutH with Right-TALE-TadA8e(V106W)) (FIG. 7F), MT-RNR2-targeting mitoABE^{MutH} (Left-TALE-TadA8e(V106W) with Right-TALE-MutH) (FIG. 7G), MT-ND1-targeting mitoABE^{Nt.BspD6I(C)} (Left-TALE-Nt.BspD6I(C) with Right-TALE-TadA8e(V106W)) (FIG. 7H), MT-ND1-targeting mitoABE^{Nt.BspD6I(C)} (Left-TALE-TadA8e(V106W) with Right-TALE-Nt.BspD6I(C)) (FIG. 7I), MT-ND4-targeting mitoCBE^{MutH} (Left-TALE-rAPOBEC1-2×UGI with Right TALE-MutH) (FIG. 7J), MT-RNR2-targeting mitoCBE^{MutH} (Left-TALE-MutH with Right-TALE-rAPOBEC1-2×UGI) (FIG. 7K). FIG. 7L shows the deep sequencing average coverage of the mitochondrial genome. FIG. 7M shows the deep sequencing average coverage of the nuclear genome. FIG. 7N shows the nuclear genome average frequency of each unique single nucleotide variant (SNV) are shown for the EGFP group (Control), non-targeting groups and targeting groups. FIGS. 7A-7K and 7N, all data are three or more biological replicates, the arrow points to the targeted editing site and the grey dots represent the editing efficiency of adenines or cytosines in the editing window. FIG. 7O shows the copy number of mtDNA that was detected by qPCR. All data points from n=3 biologically independent experiments are shown. FIGS. 7P-7W show the average frequency and mitochondrial genome position of each unique single nucleotide variant (SNV) for MT-ND1-targeting monomeric mitoABE^{MutH} (TALE-MutH-TadA8e(V106W)) (FIG. 7P), MT-ND4-targeting monomeric mitoABE^{MutH} (TALE-MutH-TadA8e(V106W)) (FIG. 7Q), MT-ND1-targeting monomeric mitoABE^{MutH} (TALE-TadA8e(V106W)-MutH) (FIG. 7R), MT-ND4-targeting monomeric mitoABE^{MutH} (TALE-TadA8e(V106W)-MutH) (FIG. 7S), MT-ND1-targeting monomeric mitoABE^{Nt.BspD6I}(C) (TALE-Nt.BspD6I(C)-TadA8e(V106W)) (FIG. 7T), MT-ND4-targeting monomeric mitoABE^{Nt.BspD6I(C)} (TALE-Nt.BspD6I(C)-TadA8e (TALE-TadA8e(V106W)) (FIG. 7U), MT-NDe-targeting monomeric mitoABENt.BspD6I(C) (TALE-TadA8e(V106W)-Nt.BspD6I(C)) (FIG. 7V), MT-ND4-targeting monomeric mitoABE^{Nt.BspD6I(C)} (TALE-TadA8e(V106W)-Nt.BspD6I(C)) (FIG. 7W). FIGS. 7P-7W show three or more biological replicates. The arrow points to the targeted editing site and the grey dots represent the editing efficiency of adenines in the editing window. FIG. 7X shows the average frequency and mitochondrial genome position of each unique single nucleotide variant (SNV) for MT-ND4-targeting DdCBE (Left TALE-DddA-G1397-N with Left TALE-DddA-G1397-C). FIG. 7Y shows the average frequency and mitochondrial genome position of each unique single nucleotide variant (SNV) for MT-RNR2-targeting DdCBE (Left TALE-DddA-G1397-N with Left TALE-DddA-G1397-C). FIGS. 7Z-HH show the analysis of indels using high throughput sequencing data for untreated HEK293T cells (FIG. 7Z) and HEK293T cells treated with nontargeting mitoABE^{MutH} (FIG. 7AA), nontargeting mitoABE^{Nt.BspD6I(C)} (FIG. 7BB), MT-ND4-targeting mitoABE^{MutH} (Left-TALE-MutH with Right-TALE-TadA8e(V106W)) (FIG. 7CC), MT-ND4-targeting mitoABE^{MutH} (Left-TALE-TadA8e(V106W) with Right-TALE-MutH) (FIG. 7DD), MT-RNR2-targeting mitoABE^{MutH} (Left-TALE-MutH with Right-TALE-TadA8e(V106W)) (FIG. 7EE), MT-RNR2-targeting mitoABE^{MutH} (Left-TALE-TadA8e(V106W) with Right-TALE-MutH) (FIG. 7FF), MT-ND1-targeting mitoABE^{Nt.BspD6I(C)} (Left-TALE-Nt.BspD6I© with Right-TALE-TadA8e(V106W)) (FIG. 7GG), MT-ND1-targeting mitoABE^{Nt.BspD6I(C)} (Left-TALE-TadA8e(V106W) with Right-TALE-Nt.BspD6I(C)) (FIG. 7HH). FIG. 7HI shows the detection of indels in mtDNA deletion by long range PCR.

FIG. 8A shows an overview of circRNA-encoded mitoABE^{MutH}-transfected cells. FIG. 8B shows editing efficiency of mitoABE^{MutH} encoded by two circular RNAs which were transfected into different cell lines to achieve top strand-specific editing. Genomic DNA was collected 2 days post-transfection. All data points from n=3 biologically independent experiments are shown. FIG. 8C shows editing efficiency of mitoABE^{MutH} encoded by two circular RNAs which were transfected into different cell lines to achieve bottom strand-specific editing. Genomic DNA was collected 2 days post-transfection. All data points from n=3 biologically independent experiments are shown. FIG. 8D shows an overview of circRNA-encoded mitoABE_{Nt.BspD6I(C)}-transfected HEK293T cells and genomic DNA collected 2 days post-transfection. FIG. SE shows the editing efficiencies of circRNA-encoded mitoABE^{Nt.BspD6I(C)} targeted the start codons of MT-ND4. All data points from n=3 biologically independent experiments are shown. FIG. 8F shows the editing efficiencies of circRNA-encoded mitoABE^{Nt.BspD6I(C)} targeted the start codons of MT-CYB and MT-CO1. All data points from n=3 biologically independent experiments are shown. FIG. 8G shows the ATP levels of cells transfected with circRNA-encoded mitoABE^{Nt.BspD6I(C)} targeted the start codon of MT-ND4. All data points from n=3 biologically independent experiments are shown. FIG. 8H shows the ATP levels of cells transfected with circRNA-encoded mitoABE^{Nt.BspD6I(C)} targeted the start codons of MT-CYB and MT-CO1. All data points from n=3 biologically independent experiments are shown. FIG. 8I shows the oxygen consumption rate (OCR) in HEK293T cells treated with circRNA-encoded mitoABENt.BspD6I(C) targeted the start codon of MT-ND4 for 2 days. All data points from n=3 biologically independent experiments are shown. FIG. 8J shows an overview of the circRNA-encoded mitoABE^{Nt.BspD6I(C)} system. GM10742 LHON disease cells were transfected with mitoABE^{Nt.BspD6I(C)}, and genomic DNA was collected 3 days post-transfection for analysis. FIG. 8K shows the editing efficiency of mitoABE^{Nt.BspD6I(C)} for correction of the 11778G>A mutation causing LHON disease in GM10742 cells. All data points from n=3 biologically independent experiments are shown. FIG. 8L shows the ATP levels of cells transfected with circRNA-encoded mitoABE^{Nt.BspD6I(C)} targeting the 11778G>A mutation causing LHON disease in GM10742 cells. All data points from n=3 biologically independent experiments are shown. FIG. 8M shows OCR of the LHON disease cell line GM10742 treated with circRNA-encoded mitoABE^{Nt.BspD6I(C)} targeting the 11778G>A mutation for 2 days. All data points from n=3 biologically independent experiments are shown. FIG. 8N shows types of mitochondrial diseases (MITOMAP) and the proportion of diseases that can theoretically be treated by mitoBEs.

### DETAILED DESCRIPTION

Provided herein, in some aspects, are systems for editing double-stranded DNA, including strand- and base-specific editing of double-stranded mitochondrial DNA in humans. In certain provided aspects, the systems for editing DNA taught herein use a single-stranded (ss-) nickase to introduce a nick in double-stranded (ds) DNA, and then a deaminase can catalyzes a desired base change which is retained on the non-nicked strand of the dsDNA. Various configurations for the systems described herein are possible. For example, in some embodiments, the description is directed to a nucleobase editor system comprising: a first unit comprising a first dsDNA binding polypeptide associated with a ss-nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase relative to an editing region on the dsDNA to effect a desired base edit. In some embodiments, the nucleobase editor system comprises: a first unit comprising a first dsDNA binding polypeptide associated with a ss-nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase such that a site action for the ss-nickase and a site of action for the deaminase are within an editing region on the dsDNA. In other aspects, provided here are components of the systems for editing DNA described herein, such as system encoding polynucleotides and novel deaminases, and methods of use thereof, such as methods of treating.

The disclosed provided herein is based on the inventors' unique perspective and unexpected findings regarding nucleobase editor systems comprising a sequence specific, single-stranded DNA nickase and a deaminase, including base-specific deaminases, that provide efficient and specific DNA base editing. Such nucleobase editor systems can be delivered to and function in a mitochondrion, and thus are capable of editing mitochondrial DNA, including human mitochondrial DNA. As demonstrated herein, the nucleobase editor systems described herein are specific enough to target single loci within the mitochondrial genome. In certain aspects demonstrated herein, it is unexpectedly shown that nucleobase editor systems allow for the assembly of a precise, strand-specific A-to-G or C-to-T, e.g., mitochondrial DNA editing system. In some embodiments, strand specificity is provided based on the unexpected finding that the distance between the dsDNA binding polypeptide binding sequence and nickase target sequence could control which DNA strand was nicked. It was found that subsequent deamination events were only retained on the un-nicked DNA strand after repair and mitochondrial DNA replication. Moreover, the nucleobase editing systems described herein can be arranged in different configurations (e.g., monomeric versus dimeric) depending on the application and desired editing, can be effectively delivered to the cell and features therein such as a mitochondrion (e.g., dimeric configurations generally have smaller units and thus less payload to deliver), and have the potential to address all known A·T to G·C and C·G to T·A disease-associated mtDNA mutations. Such capabilities greatly expand the scope and safety of current DNA editing technology and offer a way to perform programable and precise mitochondrial DNA editing.

As demonstrated herein, nucleobase editor systems for strand specific mitochondrial DNA editing offer a significant reduction in the rates of off-target editing events over previous systems. This provides a clinically significant improvement in safety profile. Moreover, by avoiding the creation of double-stranded mitochondrial DNA brakes, the nucleobase editor systems provided herein avoid rapid degradation of mitochondrial DNA or largescale deletions and rearrangements within the mitochondrial genome. Furthermore, many human disorders are characterized by mitochondrial dysfunction due to mutations within mitochondrial DNA. Currently, several molecular characteristics of mitochondria have limited the development of therapies for mitochondrial dysfunction. For example, each cell in an organism contains multiple copies of mtDNA which could be either identical in sequence or a mixture of distinct mutations. The effectiveness of pharmacologic therapies to mitochondrial disease has been limited by the proportion of mitochondria targeted. The presently disclosed methodology, through the use of mitochondrial targeting signals, can localize the mitochondrial DNA editing system homogeneously throughout the cell. Together, these unexpected safety improvements yield a mitochondrial DNA editing system suitable for precise genome targeting in humans. Moreover, the nucleobase editor systems provided herein can be used to treat mitochondrial diseases with homogeneous mutation where no wild-type mtDNA is present.

Additionally, the nucleobase editor systems taught herein provide tools for the generation of novel mitochondrial disease models. Such models would allow for significant advancements in clinical human mitochondrial disease research, as this field lacks sophisticated tools to assess the impact of specific mitochondrial DNA mutations on cell function and organismal development.

Thus, in some aspects, provided herein is a nucleobase editor system comprising: a first unit comprising a first dsDNA binding polypeptide associated with a ss-nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase such that a site action for the ss-nickase and a site of action for the deaminase are within an editing region on the dsDNA.

In other aspects, provided herein is a nucleobase editor system comprising: a ss-nickase; a deaminase; and a dsDNA binding polypeptide, wherein the ss-nickase, the deaminase, and dsDNA binding polypeptide form a complex configured such that the dsDNA binding polypeptide, when associated with a dsDNA, positions the ss-nickase and the deaminase such that a site action for the ss-nickase and a site of action for the deaminase are within an editing region on the dsDNA.

In other aspects, provided herein is a non-naturally occurring polynucleotide encoding: a first unit comprising a first dsDNA binding polypeptide associated with a ss-nickase; and/or a second unit comprising a second dsDNA binding polypeptide associated with a deaminase.

In other aspects, provided herein is a non-naturally occurring polynucleotide encoding: a first unit comprising a first dsDNA binding polypeptide associated with a ss-nickase; and a second unit comprising a second dsDNA binding polypeptide associated with a deaminase, wherein the first unit and second unit, when expressed, are configured such that the first dsDNA binding polypeptide of the first unit and the second dsDNA binding polypeptide of the second unit, when associated with a dsDNA, position the ss-nickase and the deaminase such that a site of action for the ss-nickase and a site of action for the deaminase are within an editing region on the dsDNA.

In other aspects, provided herein is a non-naturally occurring polynucleotide encoding: a ss-nickase; a deaminase; and a dsDNA binding polypeptide, wherein the ss-nickase, the deaminase, and dsDNA binding polypeptide, when expressed, form a complex configured such that the dsDNA binding polypeptide, when associated with a dsDNA, positions the ss-nickase and the deaminase such that a site action for the ss-nickase and a site of action for the deaminase are within an editing region on the dsDNA.

In other aspects, provided herein is a method of editing a target nucleotide in an editing region in a cell, the method comprising delivering a nucleobase editor system described herein or the polynucleotide encoding the nucleobase editor system described herein to the cell.

In other aspects, provided herein is a method of treating an individual having a disease associated with a DNA mutation, the method comprising administering one or more polynucleotides encoding: a first unit comprising a first dsDNA binding polypeptide associated with a ss-nickase; and a second unit comprising a second dsDNA binding polypeptide associated with a deaminase, wherein the first unit and second unit, when expressed in the individual, are configured such that the first dsDNA binding polypeptide of the first unit and the second dsDNA binding polypeptide of the second unit, when associated with a dsDNA, position the ss-nickase and the deaminase such that a site of action for the ss-nickase and a site of action for the deaminase are within an editing region on the dsDNA.

In other aspects, provided herein is a kit for a nucleobase editing system, the kit comprising: a first unit comprising a first dsDNA binding polypeptide associated with a ss-nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase such that a site of action for the ss-nickase and a site of action for the deaminase are within an editing region on the dsDNA.

In other aspects, provided herein is a kit for a nucleobase editor system, the kit comprising one or more polynucleotides encoding: a first unit comprising a first dsDNA binding polypeptide associated with a ss-nickase; and a second unit comprising a second dsDNA binding polypeptide associated with a deaminase, wherein the nucleobase editor system, when expressed, is configured such that the first dsDNA binding polypeptide of the first unit and the second dsDNA binding polypeptide of the second unit, when associated with a dsDNA, position the ss-nickase and the deaminase such that a site of action for the ss-nickase and a site of action for the deaminase are within an editing region on the dsDNA.

In other aspects, provided herein is a non-naturally occurring polypeptide having nickase activity, the non-naturally occurring polypeptide comprising the amino acid sequence of SEQ ID NO:1 with the following mutations of E91A and F94A (SEQ ID NO:2).

In other aspects, provided herein is a non-naturally occurring polypeptide comprising the amino acid sequence of SEQ ID NO:2.

In other aspects, provided herein is a polynucleotide encoding the non-naturally occurring polypeptide of SEQ ID NO:2.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The term “polynucleotide,” as used herein, refers to a polymeric form of nucleotides of any length, either ribonucleotides (RNA) or deoxyribonucleotides (DNA). Thus, this term includes, but is not limited to unless specifically stated to be so limited, single-, double- or multi-stranded DNA or RNA, genomic DNA, mitochondrial DNA (mtDNA), cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

As used herein, “treatment” or “treating” is an approach for obtaining beneficial or desired results, including clinical results. For purposes of this invention, beneficial or desired clinical results include, but are not limited to, alleviating one or more symptoms of a disease associated with a DNA mutation, e.g., a mitochondrial disease, reducing one or more symptoms of a disease, preventing one or more symptoms of a disease, treating one or more symptoms of a disease, ameliorating one or more symptoms of a disease, delaying onset of one or more symptoms associated with having a disease, diminishing the extent of one or more symptoms of a disease, stabilizing the disease (e.g., preventing or delaying the worsening of the disease), delaying or slowing the progression of the disease, ameliorating one or more symptoms of a disease, decreasing the dose of one or more other medications and/or treatments required to treat the disease, increasing the quality of life of the individual, and/or prolonging survival of the individual. Also encompassed by “treatment” is a reduction of a pathological consequence of a disease associated with a DNA mutation, e.g., a mitochondrial disease. The methods of the invention contemplate any one or more of these aspects of treatment.

The term “individual” refers to a mammal and includes, but is not limited to, human, bovine, horse, feline, canine, rodent, or primate. In some embodiments, the individual is human.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. Nucleobase Editor Systems

In certain aspects, provided herein are nucleobase editor systems for editing double-stranded DNA, including strand- and base-specific editing of mitochondrial DNA in humans. In some embodiments, the nucleobase editor systems taught herein comprise a single stranded (ss-) nickase, a deaminase, and one or more double-stranded (ds) DNA binding polypeptides, wherein the components of the system are configured such that the ss-nickase and deaminase are brought into proximity of an editing region to catalyze, at least in part, a desired nucleotide base edit. In some embodiments, the nucleobase editor system is configured to edit dsDNA in a strand specific manner. In some embodiments, the nucleobase editor system is configured as a two polypeptide system, e.g., a first polypeptide comprising a first double-stranded (ds) DNA binding polypeptide and a single-stranded (ss-) nickase; and a second polypeptide comprising a second dsDNA binding polypeptide and a deaminase. In some embodiments, the nucleobase editor system is configured to as an adenine base editing system, e.g., the deaminase converts adenine to guanine. In some embodiments, the nucleobase editor system is configured to as a cytosine base editing system, e.g., the deaminase converts cytosine to thymine. In some embodiments, the dsDNA binding polypeptide is a transcription activator-like (TAL) effector. In some embodiments, the ss-nickase recognizes a palindromic recognition sequence. In some embodiments, the ss-nickase recognizes a non-palindromic recognition sequence. In some embodiments, the ss-nickase is selected from the group consisting of MutH, MutH* (SEQ ID NO:2), and BspD6I, or a derivative thereof such as BspD6I(C). In some embodiments, the dsDNA is mitochondrial DNA. In some embodiments, the dsDNA is mitochondrial genomic DNA.

Certain aspects of the nucleobase editor systems taught herein are discussed in more detail in a modular fashion below. One of ordinary skill in the art will readily understand how the aspects of the present description can be combined to obtain any nucleobase editor system encompassed by the teachings provided herein. The discussion of nucleobase editor systems, including components and configurations thereof, in a modular fashion does not limit the scope of the description encompassed herein.

### A. Single-Stranded Nickases

The nucleobase editor systems provided herein comprise one or more single-stranded (ss-) nickases. As described herein, the ss-nickases are a polypeptide having single strand DNA nicking enzymatic activity, such as to nick a single strand of a double-stranded DNA molecule. The ss-nickase can be a full-length nickase or can be a portion of a ss-nickase comprising a functional domain catalyzing the DNA nick, e.g., is a portion of a naturally occurring nickase, or derivative thereof, having nickase activity. In some embodiments, the ss-nickase has high specificity for nicking at a specific location relative to a recognition sequence, such as at or near a recognition sequence. In some embodiments, the ss-nickase does not nick at a specific location relative to a recognition sequence, e.g., the ss-nickase comprises a domain for cleavage and does not comprise a domain for recognizing a DNA sequence. In some embodiments, ss-nickase retains nickase activity but does not bind to a recognition sequence with high specificity. In some embodiments, where the ss-nickase does not bind to a recognition sequence, the site of action of the nickase is dependent on the dsDNA binding polypeptide positioning the ss-nickase at the correct DNA location. In certain aspects of the description, the site of the nick created by the ss-nickase is referred to as the site of action of the ss-nickase.

In some embodiments, the ss-nickase recognizes a recognition sequence present in the editing region of dsDNA. In some embodiments, the ss-nickase recognizes a recognition sequence near the editing region of dsDNA, e.g., the ss-nickase recognizes a recognition sequence and then nicks at a certain distance away from the recognition sequence. In some embodiments, the recognition sequence is present on both strands of the dsDNA, e.g., as present in a palindromic recognition sequence. In some embodiments, the recognition sequence is present on a single strand of the dsDNA. In some embodiments, the recognition sequence of the ss-nickase is a palindromic recognition sequence. In some embodiments, the recognition sequence of the ss-nickase is 5′-GATC-3′, where the ss-nickase cleaves on the 5′ side of the guanine. In some embodiments, the recognition sequence of the ss-nickase is a non-palindromic recognition sequence. In some embodiments, the recognition sequence of the ss-nickase is 5′-GATD-3′, where D is a base selected from the group consisting of G, A, and T, and wherein the ss-nickase cleaves on the 5′ side of the guanine. In some embodiments, the recognition sequence of the ss-nickase is 5′-GAGTC-3′, where the ss-nickase cleaves only the strand having the recognition site at a distance of four nucleotides downstream of the recognition site toward the 3′-terminus.

In some embodiments, the recognition sequence of the ss-nickase is unmethylated. In some embodiments, the recognition sequence of the ss-nickase is methylated. In some embodiments, the recognition sequence of the ss-nickase is hemimethylated.

The nucleobase editor systems described herein can be configured with strand specificity. In some embodiments, stand specificity is conferred based on positioning of the ss-nickase relative to the ss-nickase recognition site, such as designed by the binding site of the dsDNA binding polypeptide and association of the dsDNA binding polypeptide and the ss-nickase (e.g., a linker). In some embodiments, the site of action of the ss-nickase is on the same strand of the dsDNA that is bound by the dsDNA binding polypeptide associated with the ss-nickase. In some embodiments, the dsDNA binding polypeptide associated with the ss-nickase binds the dsDNA 5-9 bases away from the site of action of the ss-nickase. In some embodiments, the site of action of the ss-nickase is on the opposite strand of the dsDNA that is bound by the dsDNA binding polypeptide associated with the ss-nickase. In some embodiments, the dsDNA binding polypeptide associated with the ss-nickase binds 0-4 bases away from the site of action of the ss-nickase.

In some embodiments, the ss-nickase is heterologous. In some embodiments, the ss-nickase of the first unit is a type I nickase, type II nickase, type III nickase, or a type IV nickase. In some embodiments, the ss-nickase is MutH or Nt.BspD6I, or a nickase derived therefrom. In some embodiments, the ss-nickase is MutH* (SEQ ID NO:2). In some embodiments, the ss-nickase is Nt.BspD6I(C) (SEQ ID NO:3). In some embodiments the nickase is FokI-FokI (D450A) (SEQ ID NO:4), Nb.BsaI (C, N441D/R442G) (SEQ ID NO:5), Nt.BsaI (C, R236D) (SEQ ID NO:6), Nb.BsmBI (C, R438D) (SEQ ID NO:7), Nt.BsmAI (C, R221D) (SEQ ID NO:8), Nb.BsrDI (C) (SEQ ID NO:9), Nt.CviPII (SEQ ID NO:10), BspQI (C) (SEQ ID NO:11), N.AlwI (C) (SEQ ID NO:12), Nt.BsrDI (SEQ ID NO:13), Nt.BtsI (SEQ ID NO:14), or I-TEV-I (SEQ ID NO:15). In some embodiments, the nickase comprises (e.g., is) the small subunit of BspD6I (ss.BspD6I) (SEQ ID NO:16), the small subunit of BsrDI (ss.BsrDI) (SEQ ID NO:17), or the small subunit of BtsI (ss.BtsI) (SEQ ID NO:18). In some embodiments, the ss-nickase is a ss-nickase reported in Desai & Shankar, FEMS Microbiology Reviews, 26, 2003, which is incorporated herein by reference in its entirety, such as S1 nuclease, P1 nuclease, Mycelia, Conidia, Slow form (S) BAL 31 nuclease, Fast form (F) BAL 31 nuclease, α U. Maydis nuclease, β U. Maydis nuclease, nuclease Bhl, aspergillus nuclease, Physarum nuclease, SP nuclease, mung bean nuclease, wheat chloroplast nuclease, nuclease I, pea seeds nuclease, tobacco nuclease I, acid nuclease of Alfalfa seedling, neutral nuclease of Alfalfa seedling, SK nuclease, hen liver nuclease, rat liver nuclei nuclease, and mouse mitochondria nuclease.

### B. Deaminases

The nucleobase editor systems provided herein comprise one or more deaminases. As described herein, the deaminases are a polypeptide having nucleotide base conversion enzymatic activity, such as to convert one nucleotide base to another, e.g., adenine (A) to guanine (G). The deaminase can be a full-length deaminase or can be a portion of a deaminase comprising a functional domain catalyzing the nucleotide base conversion, e.g., is a portion of a naturally occurring deaminase, or derivative thereof, having nucleotide base conversion activity. In some embodiments, the deaminase has high specificity for converting one nucleotide base type. In certain aspects of the description, the desired converted nucleotide base that is retained following editing (e.g., the targeted nucleotide base to be edited) is referred to as the site of action of the deaminase.

In some embodiments, the site of action of the deaminase is on the non-nicked strand of the dsDNA. For instance, as described herein, the strand of dsDNA that is not nicked retains the edited nucleotide base, and as such the strand of dsDNA that is not nicked will comprise the deaminase site of action. In some embodiments, the deaminase is heterologous. In some embodiments, the deaminase is a single-stranded deaminase (ss-deaminase). In some embodiments, wherein the deaminase is a cytosine-to-uracil deaminase, a 5-methylcytosine-to-thymine deaminase, a guanine-to-xanthine deaminase, an adenine-to-hypoxanthine deaminase, or an adenine-to-inosine deaminase.

Many deaminases are known in the art suitable for the nucleobase editor systems described herein. In some embodiments, the deaminase is TadA8e, APOBEC, or AID, or a derivative thereof. In some embodiments, the deaminase is TadA8e(V106W) (SEQ ID NO: 19). In some embodiments, the deaminase comprises (e.g., is) TadA-DE, TadA-CDa, TadA-CDa(V106W), evoAPOBEC1, evoCDA1, or evoFERNY. In some embodiments, the deaminase is APOBEC-1, APOBEC-2, APOBEC-3A, APOBEC-3B, APOBEC-3C, APOBEC-3E, APOBEC-3F, APOBEC-3G, APOBEC-3H, or APOBEC-4. In some embodiments, the deaminase is AICDA, CDA, DCTD, AMPD1, ADAT, ADAR, ADARB1, ADA, GDA, TadA, ecTadA, pCDM or ABE8, or a derivative thereof. In some embodiments, the deaminase is paired with another functional component to perform the desired nucleotide base conversion. For example, in some embodiments, the nucleobase editor system comprises a deaminase, such as APOBEC1, with a uracil glycosylase inhibitor (UGI) such as to perform C-to-T editing. For example, in some embodiments, the nucleobase editor system comprises a deaminase, such as APOBEC1, with a Uracil-DNA glycosylase (UNG) such as to perform C-to-G or C-to-A editing (rAPOBEC1-2×UGI; SEQ ID NO: 20). For example, in some embodiments, the nucleobase editor system comprises a deaminase, such as APOBEC1, such as to perform C-to-G or C-to-A editing.

### C. Double-Stranded DNA Binding Polypeptides

The nucleobase editor systems provided herein comprise one or more double-stranded (ds) DNA binding polypeptides. As described herein, the dsDNA binding polypeptides comprise a polypeptide configured to bind to dsDNA, such as at a specific location. As described in more detail below, the dsDNA binding polypeptide can be configured (e.g., is programmable) to bind to a specific location, including strand, of dsDNA relative to the desired editing region containing the nucleotide base for editing. In some embodiments, the dsDNA binding polypeptide is a TALE or zinc finger, or a portion thereof capable of binding to the dsDNA. In other embodiments, the dsDNA binding polypeptide comprises a domain of a CRISPR-Cas protein (e.g., Cas9) that binds DNA. In some embodiments, the dsDNA binding polypeptide is a catalytically inactive form of a nickase, such as an endonuclease.

In some embodiments, the nucleobase editor system comprises more than one dsDNA binding polypeptide. In such embodiments, the nucleobase editor system may comprise the same type of dsDNA binding polypeptide (e.g., TALE) or more than one different type of dsDNA binding polypeptide (e.g., TALE and zinc finger).

The nucleobase editor systems described herein may be configured in various formats, wherein the ss-nickase and/or deaminase approach an editing region from different directions. For example, in some embodiments, wherein the nucleobase editor system comprises a first unit comprising a first dsDNA binding polypeptide associated with a ss-nickase, the first dsDNA binding polypeptide of the first unit specifically associates with a portion of the dsDNA upstream of the editing region on a dsDNA sequence. In some embodiments, wherein such nucleobase editor system comprises a second dsDNA binding polypeptide associated with a deaminase, the second dsDNA binding polypeptide of the second unit specifically associates with a portion of the dsDNA downstream of the editing region on the dsDNA sequence. In some embodiments, the second dsDNA binding polypeptide binds to the stand of the dsDNA not bound by the first dsDNA binding polypeptide of the first unit.

In some embodiments, wherein the nucleobase editor system comprises a first unit comprising a first dsDNA binding polypeptide associated with a ss-nickase, the first dsDNA binding polypeptide of the first unit specifically associates with a portion of the dsDNA downstream of the editing region on a dsDNA sequence. In some embodiments, wherein such nucleobase editor system comprises a second dsDNA binding polypeptide associated with a deaminase, the second dsDNA binding polypeptide of the second unit specifically associates with a portion of the dsDNA upstream of the editing region on the dsDNA sequence. In some embodiments, the second dsDNA binding polypeptide binds to the stand of the dsDNA not bound by the first dsDNA binding polypeptide of the first unit.

As described elsewhere herein, the dsDNA binding polypeptide(s) of a nucleobase editor system may be configured to position an associated ss-nickase and/or deaminase relative to an editing region and/or site of action. In some embodiments, such position may change the editing capabilities of a nucleobase editing system. In some embodiments, additional adjustments may be included for configuring the position of the ss-nickase and/or deaminase, such as by adjusting a linker length connecting a dsDNA binding polypeptide and a ss-nickase. The present disclosure encompasses such variations of the nucleobase editor systems described herein, e.g., many variations of a nucleobase editor system may be designed based on the teachings provided herein that perform the same DNA edit. In some embodiments, the dsDNA binding polypeptide is configured to bind 0-25 base pairs away from an editing region and/or site of action of a ss-nickase or deaminase.

In some embodiments, the dsDNA binding polypeptide(s) of a nucleobase editor system may be configured to position an associated ss-nickase and/or deaminase to a specific location on the mitochondrial genome. In some embodiments, the specific location in the mitochondrial genome is within the MT-ATP6, MT-ATP8, MT-CO), MT-CO2, MT-CO3, MT-CYB, MT-ND), MT-ND2, MT-ND3, MT-ND4, MT-ND4L, MT-ND5, or MT-ND6 gene. In some embodiments, the specific location in the mitochondrial genome is within a mitochondrial ribosomal ribonucleic acid (rRNA) or transfer RNA (tRNA) gene. In some embodiments, the specific location in the mitochondrial genome is within a mitochondrial short peptide gene such as MT-RNR2 (humanin), MOTS-c, or gau.

D. Configurations of Units Comprising a Single-Stranded Nickases and/or Deaminase

As described herein, the nucleobase editor systems are configured such that a ss-nickase and deaminase are brought into proximity of an editing region to catalyze, at least in part, a desired nucleotide base edit. In some embodiments, the nucleobase editor systems comprise a single stranded (ss-) nickase, a deaminase, and one or more double-stranded (ds) DNA binding polypeptides, wherein such components may be configured in a multitude of different configurations (such as monomeric or dimeric configurations) capable of performing the desired nucleotide base edit, all of which are encompassed by the description provided herein. As described herein, following nicking and nucleotide base conversion, DNA repair mechanisms, such as performed by endogenous DNA repair proteins, may be involved in aspects of forming the final edited dsDNA.

In some embodiments, provided herein is a nucleobase editor system, comprising: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase such that a site action for the ss-nickase and a site of action for the deaminase are within an editing region on the dsDNA. Such embodiment is an example of a dimeric configuration. In some embodiments, the dimeric nucleobase editor system comprises the ss-nickase and the deaminase on separate molecules.

In some embodiments, provided is a nucleobase editor system comprising: a single-stranded (ss-) nickase; a deaminase; and a double-stranded (ds) DNA binding polypeptide, wherein the ss-nickase, the deaminase, and dsDNA binding polypeptide form a complex configured such that the dsDNA binding polypeptide, when associated with a dsDNA, positions the ss-nickase and the deaminase such that a site action for the ss-nickase and a site of action for the deaminase are within an editing region on the dsDNA. Such embodiment is an example of a monomeric configuration. In some embodiments, the monomeric nucleobase editor system comprises the ss-nickase and deaminase on a single molecule.

In some embodiments, provided is a nucleobase editor system configured for editing two or more nucleotide bases, e.g., nucleotide bases at different DNA locations. In some embodiments, when the nucleobase editor system is configured for editing a plurality of nucleotide bases, at least two or more nucleotide bases of the plurality are in different editing regions. Various configurations of nucleobase editor systems described herein can be used to perform such editing. For example, in some embodiments, the nucleobase editor system configurated for editing a plurality of nucleotide bases comprises a first unit comprising a first double-stranded (ds) DNA binding polypeptide associated with a first single-stranded (ss-) nickase; a second unit comprising a second dsDNA binding polypeptide associated with a second ss-nickase, wherein the first dsDNA binding polypeptide and the second dsDNA binding polypeptide recognize different recognition sequences; a third unit comprising a third dsDNA binding polypeptide associated with a first deaminase; and a fourth unit comprising a fourth dsDNA binding polypeptide associated with a second deaminase, wherein the nucleobase editor system is configured such that the first dsDNA binding polypeptide of the first unit and the third dsDNA binding polypeptide of the third unit, when associated with a dsDNA, position the first ss-nickase and the first deaminase such that a site action for the first ss-nickase and a site of action for the first deaminase are within a first editing region on the dsDNA, and wherein the nucleobase editor system is configured such that the third dsDNA binding polypeptide of the third unit and the fourth dsDNA binding polypeptide of the fourth unit, when associated with the dsDNA, position the second ss-nickase and the second deaminase such that a site action for the second ss-nickase and a site of action for the second deaminase are within a second editing region on the dsDNA. In some embodiments, the first ss-nickase and the second ss-nickase recognize different recognition sequences. In some embodiments, the first deaminase and the second deaminase are the same. In some embodiments, the first deaminase and the second deaminase catalyze the same nucleotide base conversions. In some embodiments, the first deaminase and the second deaminase catalyze different nucleotide base conversions.

In some embodiments, the nucleobase editor systems comprise many components, such as a dsDNA binding polypeptide and a ss-nickase, that while they can be described separately herein are configured as a unit when performing the desired editing. Such components can be associated via numerous ways, such as via direct fusion (e.g., as a single expressed polypeptide) non-covalent interaction, or covalent linkage (e.g., via a polypeptide linkers). In some embodiments, wherein the nucleobase editor system comprises a first unit comprising a first dsDNA binding polypeptide and a ss-nickase, the first unit is a fusion polypeptide, wherein the first dsDNA binding polypeptide of the first unit is fused to the ss-nickase of the first unit. In some embodiments, the first dsDNA binding polypeptide of the first unit is fused to the C-terminus of the ss-nickase of the first unit. In some embodiments, the first dsDNA binding polypeptide of the first unit is fused to the N-terminus of the ss-nickase of the first unit. In some embodiments, the first unit further comprises a linker associating the first dsDNA binding polypeptide and the ss-nickase. In some embodiments, wherein the nucleobase editor system comprises a second unit comprising a second dsDNA binding polypeptide and a deaminase, the second unit is a fusion polypeptide, wherein the second dsDNA binding polypeptide of the second unit is fused to the deaminase of the second unit. In some embodiments, the second dsDNA binding polypeptide of the second unit is fused to the C-terminus of the deaminase of the second unit. In some embodiments, the second dsDNA binding polypeptide of the second unit is fused to the N-terminus of the deaminase of the second unit. In some embodiments, the second unit further comprises a linker associating the second dsDNA binding polypeptide and the deaminase. In some embodiments, the linker of the first unit and/or the linker of the second unit comprise a polypeptide linker. In some embodiments, the polypeptide linker is from 1-100 amino acids in length, such as any of 1-60 amino acids in length, 1-50 amino acids in length, 1-40 amino acids in length, or 2-32 amino acids in length. In some embodiments, the polypeptide linker is any of the following amino acids in lengths: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, the linker comprises, including consists essentially of or consists of, GS, AEAAAKEAAAKEAAAKEAAAKA (SEQ ID NO:25), or GSGSSGGSSGSETPGTSESATPESSGGSSGGS (SEQ ID NO:26).

In some embodiments, the dsDNA binding polypeptide and a ss-nickase associate non-covalently. In some embodiments, the dsDNA binding polypeptide and a deaminase associate non-covalently.

In some embodiments, the nucleobase editor system comprises a first unit comprising, from N- to C-terminus, a dsDNA binding polypeptide (e.g., a TALE), such as from an array (e.g., a TALE N-terminal non-repeat-TALE Repeat array), a linker (e.g., a 2 amino acid linker), and a ss-nickase, and a second unit comprising, from N- to C-terminus, a dsDNA binding polypeptide (e.g., a TALE), such as from an array (e.g., a TALE N-terminal non-repeat-TALE Repeat array), a linker (e.g., a 2 amino acid linker), and a deaminase. Such embodiment is an example of a dimeric configuration.

In some embodiments, the nucleobase editor system comprises, from N- to C-terminus, a dsDNA binding polypeptide (e.g., a TALE), such as from an array (e.g., a TALE N-terminal non-repeat-TALE Repeat array), a linker (e.g., a 2 amino acid linker), a ss-nickase, a linker (e.g., a 2 amino acid linker), and a deaminase. In some embodiments, the nucleobase editor system comprises, from N- to C-terminus, a dsDNA binding polypeptide (e.g., a TALE), such as from an array (e.g., a TALE N-terminal non-repeat-TALE Repeat array), a linker (e.g., a 2 amino acid linker), a deaminase, a linker (e.g., a 2 amino acid linker), and a ss-nickase. Such embodiments are an example of monomeric configurations.

E. Editing region and double-stranded DNA type

To facilitate description of the nucleobase editing systems provided herein, the term editing region is used in reference to a region of dsDNA where the targeted (desired) ss-nickase and deaminase activity occur (the targeted sites of action of the nickase and the deaminase). Nucleotide editing can occur outside of the editing region, such as off-target editing. In some embodiments, a base pair length is provided to describe subject matter provided herein. It is noted that sites of action will occur on opposite strands (such as for the site of action for the ss-nickase and deaminase), and descriptions including base pair distance may be assessed based on a complementary dsDNA form of the DNA.

In some embodiments, the editing region on the dsDNA is 0-24 base pairs in length, including any of 1-20 base pairs in length, 1-10 base pairs in length, 10-20 base pairs in length, 10-16 base pairs in length, 14-20 base pairs in length, or 14-16 base pairs in length. In some embodiments, the site of action of the ss-nickase is no more than 10 base pairs, such as no more than any of 9 base pairs, 8 base pairs, 7 base pairs, 6 base pairs, 5 base pairs, 4 base pairs, 3 base pairs, or 1 base pairs, from the site of action of the deaminase.

In some embodiments, the nucleobase editing system is strand-biased, e.g., at least about 65%, such as at least about any of 70%, 75%, 80%, 85%, 90%, or 95%, of edits are performed on a desired strand of the dsDNA. In some embodiments, strand-specific is equivalent to strand-biased.

In some embodiments, the dsDNA targeted for editing by the nucleobase editor systems described herein can be any type of double-stranded DNA. In some embodiments, the dsDNA is a circularized dsDNA. In some embodiments, the dsDNA is mitochondrial DNA (mtDNA). In some embodiments, the mtDNA is located in a mitochondrion, such as mitochondrion in a cell including the cell in an individual. In some embodiments, the dsDNA is mitochondrial genomic DNA. In some embodiments, the dsDNA is a B-DNA conformation. In some embodiments, the dsDNA is an A-DNA conformation. In some embodiments, the dsDNA is a Z-DNA conformation.

### F. Additional Features

In certain aspects, the nucleotide editor systems provided herein may comprise one or more additional features, such as to aid in delivery and/or function of the nucleotide editor systems.

Mitochondria are unique sub-cellular organelles that possess their own DNA and RNA and mechanisms for their translation, yet they express only 10% of the proteins that they contain. Instead, mitochondria rely in part on the translation products of nuclear genes. These products traverse the cytoplasm and are ‘imported’ into the mitochondria via a system of outer- and inner-membrane-bound protein complexes, where they are delivered to the appropriate mitochondrial compartment and rendered active. This mitochondrial import process is regulated by an N-terminal pre-sequence in the nuclear gene of the protein that tags the protein with a sequence that tells the import machinery where the protein should be delivered—these are known as mitochondrial location signal (MLS), which can also be referred to as a mitochondrial targeting signal (MTS), peptides. Once the protein has been transported to the desired compartment, the MTS portion of the protein may be removed by a mitochondrial peptidase, allowing the protein to fold into its functional state and become active.

In some embodiments, the nucleobase editor system, or one or more components thereof, comprise a mitochondrial location signal (MLS), which can also be referred to as a mitochondrial targeting signal (MTS). In some embodiments, the ss-nickase is associated with a localization signal, such as a mitochondrial location signal (MLS). In some embodiments, the deaminase is associated with a localization signal, such as a mitochondrial location signal (MLS). In some embodiments, wherein the nucleobase editor system comprises more than one unit (such as a first unit comprising a dsDNA binding polypeptide and a ss-nickase and a second unit comprising a dsDNA binding polypeptide and a deaminase), any one or more, including all, units of the nucleobase editor system may comprise a MLS.

In some embodiments, wherein the nucleobase editor system comprises a first unit comprising a ss-nickase and a second unit comprising a deaminase, the first unit further comprises a mitochondrial localization signal (MLS). In some embodiments, the MLS is positioned at the N-terminus of the first unit. In some embodiments, wherein the nucleobase editor system comprises a first unit comprising a ss-nickase and a second unit comprising a deaminase, the second unit further comprises a mitochondrial localization signal (MLS). In some embodiments, the MLS is positioned at the N-terminus of the second unit.

In some embodiments, the MLS is about 10 to about 80 amino acids in length, such as about any of 10-15, 15-20, 20-25, 25-30, 30-35, 35-40, 40-45, 45-50, 50-55, 55-60, 60-65, 65-70, 70-75, or 75-80 amino acids in length.

In some embodiments, the MLS comprises an amphipathic helix structural motif. In order to adopt the amphipathic helix structural motif, the MLS can be enriched in basic (e.g., Arg, Lys), hydroxylated (e.g., Ser, Thr) and/or hydrophobic (e.g., Ala, Leu, Ile) residues. In some embodiments, the MLS comprising an amphipathic helix structural motif exhibit alternating hydrophobic and hydrophilic segments. In some embodiments, at least about 20% (such as at least about any of 30%, 40%, 50%, or 60%) of the amino acid residues in the MLS are basic amino acid residues. In some embodiments, at least about 20% (such as at least about any of 30%, 40%, 50%, or 60%) of the amino acid residues in the MLS are hydrophobic amino acid residues. In some embodiments, the MLS is amphipathic, for example forms an amphipathic helix. In some embodiments, the MLS comprises an alternating pattern of hydrophobic and basic residues. In some embodiments, the MLS is derived from a protein selected from the group consisting of ATP synthase, cytochrome C oxidase peptide VIII, Su9, and HSP60. In some embodiments, the MLS can selectively direct a compound to an outer membrane, an inner membrane, and inter-membrane space, or a mitochondrial matrix.

In some embodiments, the nucleobase editor system, when expressed as one or more polypeptides (e.g., monomeric versus dimeric forms of a nucleobase editor system described herein), has a molecular weight of less than about 150 kDa, such as less than about any of 145 kDa, 140 kDa, 135 kDa, 130 kDa, 125 kDa, 120 kDa, 115 kDa, 110 kDa, 105 kDa, 100 kDa, 95 kDa, 90 kDa, 85 kDa, 80 kDa, 75 kDa, 70 kDa, 65 kDa, 60 kDa, 55 kDa, 50 kDa, 45 kDa, or 40 kDa. In some embodiments, the unit of a nucleobase editor system, such as a first unit comprising a first double-stranded (ds) DNA binding polypeptide associated with a single-stranded (ss-) nickase or a second unit comprising a second dsDNA binding polypeptide associated with a deaminase, when expressed as one or more polypeptides, has a molecular weight of less than about 150 kDa, such as less than about any of 145 kDa, 140 kDa, 135 kDa, 130 kDa, 125 kDa, 120 kDa, 115 kDa, 110 kDa, 105 kDa, 100 kDa, 95 kDa, 90 kDa, 85 kDa, 80 kDa, 75 kDa, 70 kDa, 65 kDa, 60 kDa, 55 kDa, 50 kDa, 45 kDa, or 40 kDa.

### G. Polynucleotide Forms of the Nucleobase Editor Systems

Provided herein, in certain aspects, are polynucleotide forms of the nucleobase editor systems described herein. The disclosure provided herein covers the multitude of formats capable of introducing functional nucleobase editor systems described herein in a call, including different types of polynucleotides (e.g., DNA or RNA, such as circular RNA) and different designs of polynucleotides.

“Introducing” or “introduction” used herein in reference to delivering nucleobase editor systems means delivering one or more nucleobase editor system components, or a precursor thereof (e.g., one or more polynucleotides encoding a nucleobase editor system or a nucleobase editor system component comprising a MLS), to a cell. The methods of the present application can employ many delivery systems, including but not limited to, viral, liposome, electroporation, microinjection and conjugation, to achieve the introduction of the construct as described herein into a cell. Conventional viral and non-viral based gene transfer methods can be used to introduce nucleic acids into mammalian cells or target tissues. Such methods can be used to administer nucleic acids of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the cell. As described herein, the polynucleotides taught herein encoding a nucleobase editor system may be one or more polynucleotides. In some embodiments, introducing the nucleobase editor system may comprise introducing two or more different polynucleotides, wherein said two or more different polynucleotides may be introduced simultaneously, sequentially, or concurrently, including introduced simultaneously, sequentially, or concurrently into the cell.

Methods of non-viral delivery of one or more components of a nucleobase editing system, including nucleic acids, include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and trafficking the viral payload to the cellular nuclei. In some embodiments, delivery comprises introducing a viral vector (such as lentiviral vector) encoding the nucleic acid(s) to the cell. In some embodiments, the viral vector is an AAV, e.g., AAV8. In some embodiments, such as for delivery using an AAV, the inventors envision that monomeric forms of the nucleobase editor systems may be more suitable for packaging and as a cargo, e.g., due to the smaller size overall size of a monomer of a nucleobase editor system described herein as compared to a dimeric form of a nucleobase editor system. In some embodiments, delivery comprises introducing a plasmid encoding one or more nucleobase editing system components to the cell. In some embodiments, delivery comprises introducing (e.g., by electroporation) one or more nucleobase editing system components into the cell. In some embodiments, delivery comprises transfection of one or more nucleobase editing system components into the cell.

In some embodiments, the polynucleotide, such as the polynucleotide introduced to a cell, is DNA or RNA. In some embodiments, the RNA is linear RNA. In some embodiments, the RNA is circular RNA. In some embodiments, the linear RNA is capable of forming a circular RNA. The circulation can be performed, for example, by using the Tornado expression system (“Twister-optimized RNA for durable overexpression”) as described in Litke, J. L. & Jaffrey, S. R. Highly efficient expression of circular RNA aptamers in cells using autocatalytic transcripts. Nat Biotechnol 37, 667-675 (2019), which is hereby incorporated herein by reference in its entirety. Briefly, Tornado-expressed transcripts contain an RNA of interest flanked by Twister ribozymes. A twister ribozyme is any catalytic RNA sequences that are capable of self-cleavage. The ribozymes rapidly undergo autocatalytic cleavage, leaving termini that are ligated by an RNA ligase. Non-limiting examples of RNA ligase include: RtcB, T4 RNA Ligase 1, T4 RNA Ligase 2, Rn13 and Trl1. In some embodiments, the RNA ligase is expressly endogenously in the cell. In some embodiments, the RNA ligase is RNA ligase RtcB. In some embodiments, the method further comprises introducing an RNA ligase (e.g., RtcB) into the cell. In some embodiments, the RNA is circularized before being introduced to the cell. In some embodiments, the RNA is chemically synthesized. In some embodiments, the RNA is circularized through in vitro enzymatic ligation (e.g., using RNA or DNA ligase) or chemical ligation (e.g., using cyanogen bromide or a similar condensing agent).

In some embodiments, the polynucleotides described herein comprise additional features useful for expression of a nucleobase editor system in a cell, such as a promoter sequence.

In some embodiments, provided herein is a non-naturally occurring polynucleotide, including one or more polynucleotides, encoding a nucleobase editor system described herein. In some embodiments, provided herein is a non-naturally occurring polynucleotide, including one or more polynucleotides, encoding: a first unit comprising a first double-stranded (ds) DNA binding polypeptide associated with a single-stranded (ss-) nickase; and/or a second unit comprising a second dsDNA binding polypeptide associated with a deaminase.

In some embodiments, provided herein is a non-naturally occurring polynucleotide, including one or more polynucleotides, encoding: a first unit comprising a first double-stranded (ds) DNA binding polypeptide associated with a single-stranded (ss-) nickase; and a second unit comprising a second dsDNA binding polypeptide associated with a deaminase, wherein the first unit and second unit, when expressed, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In some embodiments, provided herein is a non-naturally occurring polynucleotide, including one or more polynucleotides, encoding: a single-stranded (ss-) nickase; a deaminase; and a double-stranded (ds) DNA binding polypeptide, wherein the ss-nickase, the deaminase, and dsDNA binding polypeptide, when expressed, form a complex configured such that the dsDNA binding polypeptide, when associated with a dsDNA, positions the ss-nickase and the deaminase with a site action for the ss-nickase and a site of action for the deaminase within an editing region on the dsDNA.

### H. Example Nucleobase Editor Systems

In some embodiments, provided is a nucleobase editor system comprising a first unit comprising a TALE associated with a single-stranded (ss-) nickase, wherein the ss-nickase is MutH; and a second unit comprising a second TALE associated with an engineered deoxyadenosine deaminase, wherein the deaminase is TadA8e(V106W). In some embodiments, the nucleobase editor system is configured such that the TALE associated MutH unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of MutH. In some embodiments, the TALE of the second unit is fused to the C-terminus of TadA8e(V106W). In some embodiments, the first unit further comprises a linker associating the TALE and MutH. In some embodiments, the second unit further comprises a linker associating the TALE and TadA8e(V106W). In some embodiments, the TALE of the first unit binds to a DNA region upstream of the MutH recognition sequence of 5′-GATC-3′. In some embodiments, the TALE of the second unit binds to a DNA region downstream of the MutH recognition sequence of 5′-GATC-3′. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is TadA8e(V106W); and a second unit comprising a second TALE associated with single-stranded (ss-) nickase, wherein the ss-nickase is MutH. In some embodiments, the nucleobase editor system is configured such that the TALE associated MutH unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of TadA8e(V106W). In some embodiments, the TALE of the second unit is fused to the C-terminus of MutH. In some embodiments, the first unit further comprises a linker associating the TALE and TadA8e(V106W). In some embodiments, the second unit further comprises a linker associating the TALE and MutH. In some embodiments, the TALE of the first unit binds to a DNA region upstream of the MutH recognition sequence of 5′-GATC-3′. In some embodiments, the TALE of the second unit binds to a DNA region downstream of the MutH recognition sequence of 5′-GATC-3′. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with a single-stranded (ss-) nickase, wherein the ss-nickase is MutH*; and a second unit comprising a second TALE associated with an engineered deoxyadenosine deaminase, wherein the deaminase is TadA8e(V106W). In some embodiments, the nucleobase editor system is configured such that the TALE associated MutH* unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of MutH*. In some embodiments, the TALE of the second unit is fused to the C-terminus of TadA8e(V106W). In some embodiments, the first unit further comprises a linker associating the TALE and MutH*. In some embodiments, the second unit further comprises a linker associating the TALE and TadA8e(V106W). In some embodiments, the TALE of the first unit binds to a DNA region upstream of the MutH* recognition sequence of 5′-GATD-3′, where D is a base selected from the group consisting of G, A, and T. In some embodiments, the TALE of the second unit binds to a DNA region downstream of the MutH* recognition sequence of 5′-GATD-3′, where D is a base selected from the group consisting of G, A, and T. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is TadA8e(V106W); and a second unit comprising a second TALE associated with single-stranded (ss-) nickase, wherein the ss-nickase is MutH*. In some embodiments, the nucleobase editor system is configured such that the TALE associated MutH* unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of TadA8e(V106W). In some embodiments, the TALE of the second unit is fused to the C-terminus of MutH*. In some embodiments, the first unit further comprises a linker associating the TALE and TadA8e(V106W). In some embodiments, the second unit further comprises a linker associating the TALE and MutH*. In some embodiments, the TALE of the first unit binds to a DNA region upstream of the MutH* recognition sequence of 5′-GATD-3′, where D is a base selected from the group consisting of G, A, and T. In some embodiments, the TALE of the second unit binds to a DNA region downstream of the MutH* recognition sequence of 5′-GATD-3′, where D is a base selected from the group consisting of G, A, and T. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with a single-stranded (ss-) nickase, wherein the ss-nickase is Nt.BspD6I(C); and a second unit comprising a second TALE associated with an engineered deoxyadenosine deaminase, wherein the deaminase is TadA8e(V106W). In some embodiments, the nucleobase editor system is configured such that the TALE associated Nt.BspD6I(C) unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of Nt.BspD6I(C). In some embodiments, the TALE of the second unit is fused to the C-terminus of TadA8e(V106W). In some embodiments, the first unit further comprises a linker associating the TALE and Nt.BspD6I(C). In some embodiments, the second unit further comprises a linker associating the TALE and TadA8e(V106W). In some embodiments, the TALE of the first unit binds to a DNA region upstream of the Nt.BspD6I(C) site of action. In some embodiments, the TALE of the second unit binds to a DNA region downstream of the Nt.BspD6I(C) site of action. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is TadA8e(V106W); and a second unit comprising a second TALE associated with single-stranded (ss-) nickase, wherein the ss-nickase is Nt.BspD6I(C). In some embodiments, the nucleobase editor system is configured such that the TALE associated Nt.BspD6I(C) unit and the TALE associated TadA8e(V106W) unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of TadA8e(V106W). In some embodiments, the TALE of the second unit is fused to the C-terminus of Nt.BspD6I(C). In some embodiments, the first unit further comprises a linker associating the TALE and TadA8e(V106W). In some embodiments, the second unit further comprises a linker associating the TALE and Nt.BspD6I(C). In some embodiments, the TALE of the first unit binds to a DNA region upstream of the Nt.BspD6I(C) site of action. In some embodiments, the TALE of the second unit binds to a DNA region downstream of the Nt.BspD6I(C) site of action. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with a single-stranded (ss-) nickase, wherein the ss-nickase is MutH; and a second unit comprising a second TALE associated with an engineered deoxyadenosine deaminase, wherein the deaminase is rAPOBEC1-2×UGI. In some embodiments, the nucleobase editor system is configured such that the TALE associated MutH unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of MutH. In some embodiments, the TALE of the second unit is fused to the C-terminus of rAPOBEC1-2×UGI. In some embodiments, the first unit further comprises a linker associating the TALE and MutH. In some embodiments, the second unit further comprises a linker associating the TALE and rAPOBEC1-2×UGI. In some embodiments, the TALE of the first unit binds to a DNA region upstream of the MutH recognition sequence of 5′-GATC-3′. In some embodiments, the TALE of the second unit binds to a DNA region downstream of the MutH recognition sequence of 5′-GATC-3′. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is rAPOBEC1-2×UGI; and a second unit comprising a second TALE associated with single-stranded (ss-) nickase, wherein the ss-nickase is MutH. In some embodiments, the nucleobase editor system is configured such that the TALE associated MutH unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of rAPOBEC1-2×UGI. In some embodiments, the TALE of the second unit is fused to the C-terminus of MutH. In some embodiments, the first unit further comprises a linker associating the TALE and rAPOBEC1-2×UGI. In some embodiments, the second unit further comprises a linker associating the TALE and MutH. In some embodiments, the TALE of the first unit binds to a DNA region upstream of the MutH recognition sequence of 5′-GATC-3′. In some embodiments, the TALE of the second unit binds to a DNA region downstream of the MutH recognition sequence of 5′-GATC-3′. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with a single-stranded (ss-) nickase, wherein the ss-nickase is MutH*; and a second unit comprising a second TALE associated with an engineered deoxyadenosine deaminase, wherein the deaminase is rAPOBEC1-2×UGI. In some embodiments, the nucleobase editor system is configured such that the TALE associated MutH* unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of MutH*. In some embodiments, the TALE of the second unit is fused to the C-terminus of rAPOBEC1-2×UGI. In some embodiments, the first unit further comprises a linker associating the TALE and MutH*. In some embodiments, the second unit further comprises a linker associating the TALE and rAPOBEC1-2×UGI. In some embodiments, the TALE of the first unit binds to a DNA region upstream of the MutH* recognition sequence of 5′-GATD-3′, where D is a base selected from the group consisting of G, A, and T. In some embodiments, the TALE of the second unit binds to a DNA region downstream of the MutH* recognition sequence of 5′-GATD-3′, where D is a base selected from the group consisting of G, A, and T. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is rAPOBEC1-2×UGI; and a second unit comprising a second TALE associated with single-stranded (ss-) nickase, wherein the ss-nickase is MutH*. In some embodiments, the nucleobase editor system is configured such that the TALE associated MutH* unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of rAPOBEC1-2×UGI. In some embodiments, the TALE of the second unit is fused to the C-terminus of MutH*. In some embodiments, the first unit further comprises a linker associating the TALE and rAPOBEC1-2×UGI. In some embodiments, the second unit further comprises a linker associating the TALE and MutH*. In some embodiments, the TALE of the first unit binds to a DNA region upstream of the MutH* recognition sequence of 5′-GATD-3′, where D is a base selected from the group consisting of G, A, and T. In some embodiments, the TALE of the second unit binds to a DNA region downstream of the MutH* recognition sequence of 5′-GATD-3′, where D is a base selected from the group consisting of G, A, and T. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with a single-stranded (ss-) nickase, wherein the ss-nickase is Nt.BspD6I(C); and a second unit comprising a second TALE associated with an engineered deoxyadenosine deaminase, wherein the deaminase is rAPOBEC1-2×UGI. In some embodiments, the nucleobase editor system is configured such that the TALE associated Nt.BspD6I(C) unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of Nt.BspD6I(C). In some embodiments, the TALE of the second unit is fused to the C-terminus of rAPOBEC1-2×UGI. In some embodiments, the first unit further comprises a linker associating the TALE and Nt.BspD6I(C). In some embodiments, the second unit further comprises a linker associating the TALE and rAPOBEC1-2×UGI. In some embodiments, the TALE of the first unit binds to a DNA region upstream of the Nt.BspD6I(C) site of action. In some embodiments, the TALE of the second unit binds to a DNA region downstream of the Nt.BspD6I(C) site of action. In some embodiments provided herein are polynucleotides encoding this nucleobase editor system. In some embodiments the first unit and second unit of the nucleobase editor system are separate single polypeptides.

In some embodiments, provided is a nucleobase editor system comprising a first unit comprising a TALE associated with an engineered deoxyadenosine deaminase, wherein the deaminase is rAPOBEC1-2×UGI; and a second unit comprising a second TALE associated with single-stranded (ss-) nickase, wherein the ss-nickase is Nt.BspD6I(C). In some embodiments, the nucleobase editor system is configured such that the TALE associated Nt.BspD6I(C) unit and the TALE associated rAPOBEC1-2×UGI unit, when associated with a dsDNA, positions the nickase and the deaminase such that a site of action for the ss-nickase and a site of action for the deaminase are within an editing region on the ds DNA, wherein the editing region is 1 to 24 bases in length. In some embodiments, the TALE of the first unit is fused to the N-terminus of rAPOBEC1-2×UGI. In some embodiments, the TALE of the second unit is fused to the C-terminus of Nt.BspD6I(C). In some embodiments, the first unit further comprises a linker associating the TALE and rAPOBEC1-2×UGI. In some embodiments, the second unit further comprises a linker associating the TALE and Nt.BspD6I(C). In some embodiments, the TALE of the first unit binds to a DNA region upstream of the Nt.BspD6I(C) site of action. In some embodiments, the TALE of the second unit binds to a DNA region downstream of the Nt.BspD6I(C) site of action. In some embodiments provided herein are polynucleotides encoding this nucleobase editor system. In some embodiments the first unit and second unit of the nucleobase editor system are separate single polypeptides.

In some embodiments, provided is a nucleobase editor system comprising a single unit comprising a TALE associated with a single-stranded (ss-) nickase, wherein the ss-nickase is MutH, and an engineered deoxyadenosine deaminase, wherein the deaminase is TadA8e(V106W). In some embodiments, the nucleobase editor system is configured such that the TALE associated MutH and TadA8e(V106W), form a complex configured such at the TALE, when associated with a dsDNA, positions the nickase and the deaminase such that a site of action for the ss-nickase and a site of action for the deaminase are within an editing region on the ds DNA, wherein the editing region is 1 to 24 bases in length. In some embodiments single polypeptide is ordered such that the TALE is fused to the N-terminus of MutH, which in turn, is fused to the N-terminus of TadA8e(V106W). In some embodiments linker polypeptides separate the TALE, MutH, and TadA8e(V106W) domains of the nucleobase editor system. In some embodiments, the TALE of the first unit binds to a DNA region upstream of the MutH recognition sequence of 5′-GATC-3′. In some embodiments provided herein are polynucleotides encoding this nucleobase editor system.

In some embodiments, provided is a nucleobase editor system comprising a single unit comprising a TALE associated with an engineered deoxyadenosine deaminase, wherein the deaminase is TadA8e(V106W), and an engineered ss-nickase, wherein the ss-nickase is MutH. In some embodiments, the nucleobase editor system is configured such that the TALE associated TadA8e(V106W) and MutH, form a complex configured such at the TALE, when associated with a dsDNA, positions the nickase and the deaminase such that a site of action for the ss-nickase and a site of action for the deaminase are within an editing region on the ds DNA, wherein the editing region is 1 to 24 bases in length. In some embodiments single polypeptide is ordered such that the TALE is fused to the N-terminus of TadA8e(V106W), which in turn, is fused to the N-terminus of MutH. In some embodiments linker polypeptides separate the TALE, MutH, and TadA8e(V106W) domains of the nucleobase editor system. In some embodiments, the TALE of the first unit binds to a DNA region upstream of the MutH recognition sequence of 5′-GATC-3′. In some embodiments provided herein are polynucleotides encoding this nucleobase editor system.

In some embodiments, provided is a nucleobase editor system comprising a single unit comprising a TALE associated with a single-stranded (ss-) nickase, wherein the ss-nickase is Nt.BspD6I(C), and an engineered deoxyadenosine deaminase, wherein the deaminase is TadA8e(V106W). In some embodiments, the nucleobase editor system is configured such that the TALE associated Nt.BspD6I(C) and TadA8e(V106W), form a complex configured such at the TALE, when associated with a dsDNA, positions the nickase and the deaminase such that a site of action for the ss-nickase and a site of action for the deaminase are within an editing region on the ds DNA, wherein the editing region is 1 to 24 bases in length. In some embodiments single polypeptide is ordered such that the TALE is fused to the N-terminus of Nt.BspD6I(C), which in turn, is fused to the N-terminus of TadA8e(V106W). In some embodiments linker polypeptides separate the TALE, Nt.BspD6I(C), and TadA8e(V106W) domains of the nucleobase editor system. In some embodiments, the TALE of the first unit binds to a DNA region upstream of the Nt.BspD6I(C) site of action. In some embodiments provided herein are polynucleotides encoding this nucleobase editor system.

In some embodiments, provided is a nucleobase editor system comprising a single unit comprising a TALE associated with an engineered deoxyadenosine deaminase, wherein the deaminase is TadA8e(V106W), and an engineered ss-nickase, wherein the ss-nickase is Nt.BspD6I(C). In some embodiments, the nucleobase editor system is configured such that the TALE associated TadA8e(V106W) and Nt.BspD6I(C), form a complex configured such at the TALE, when associated with a dsDNA, positions the nickase and the deaminase such that a site of action for the ss-nickase and a site of action for the deaminase are within an editing region on the ds DNA, wherein the editing region is 1 to 24 bases in length. In some embodiments single polypeptide is ordered such that the TALE is fused to the N-terminus of TadA8e(V106W), which in turn, is fused to the N-terminus of Nt.BspD6I(C). In some embodiments linker polypeptides separate the TALE, Nt.BspD6I(C), and TadA8e(V106W) domains of the nucleobase editor system. In some embodiments, the TALE of the first unit binds to a DNA region upstream of the Nt.BspD6I(C) site of action. In some embodiments provided herein are polynucleotides encoding this nucleobase editor system.

### III. Methods of use and making

Provided herein, in certain aspects, are methods of using the nucleobase editor systems taught herein.

In certain aspects, the methods provided herein are directed to a method of editing a target nucleotide in an editing region in a cell or a feature thereof, such as a mitochondrion. In some embodiments, the editing performed results in an edit that is not transient, e.g., remains in the edited form for at least about 10 days, such as at least about any of 15 days, 25 days, 1 month, 3 months, 6 months, 9 months, or 1 year.

In some embodiments, provided is a method of editing a target nucleotide in an editing region in a cell, the method comprising delivering any nucleobase editor system described herein to the cell (e.g., a monomeric or dimeric nucleobase editor system). In some embodiments, the nucleobase editor system, or at least a component thereof, is in a polypeptide form. In some embodiments, such polypeptide form of a nucleobase editor system, or at least a component thereof, comprises a localization signal, e.g., a mitochondrial localization signal (MLS) configured for transportation of the polypeptide(s) to one or more mitochondria. In some embodiments, the nucleobase editor system, or at least a component thereof, is in a polynucleotide form, such as one or more polynucleotides encoding the nucleobase editor system, or at least the component thereof. In such systems, the one or more polynucleotides are configured such that the associated polypeptide of the nucleobase editor system is expressed in the cell. In some embodiments, the editing region is on mitochondrial DNA.

In some embodiments, provided is a method of editing a target nucleotide in an editing region in a mitochondrion of a cell, the method comprising delivery any nucleobase editor system described herein to the cell (e.g., a monomeric or dimeric nucleobase editor system), wherein the nucleobase editor system is delivered to the cell in a polynucleotide form, and wherein the polynucleotide form of the nucleobase editor system is configured to express the nucleobase editor system in the cell. In some embodiments, the expressed nucleobase editor system comprise one or more mitochondrial localization signals (MLS) such that the nucleobase editor system is transported to the mitochondrion of the cell.

The purposes of editing a target nucleotide in a cell, such as in a mitochondrion, using the nucleobase editor systems described herein are diverse, all of which are encompassed by the description provided herein. For example, in some embodiments, the method of editing a target nucleotide in a cell using a nucleobase editor system provided herein is performed to create a cell model. In some embodiments, the cell model is a model for a mitochondrial disease. In some embodiments, the target nucleotide is the site of a known SNP, wherein the nucleobase editor system is configured to edit the target nucleotide to revert the SNP to the wild type nucleotide, create the SNP, or adjust the SNP associated with a disease to another nucleotide base.

In some embodiments, provided is a method of treating an individual having a disease associated with a DNA mutation, the method comprising administering to the individual a nucleobase editor system described herein (e.g., a monomeric or dimeric nucleobase editor system) or a precursor thereof. In some embodiments, the nucleobase editor system or precursor thereof comprises one or more polynucleotides encoding: a first unit comprising a first double-stranded (ds) DNA binding polypeptide associated with a single-stranded (ss-) nickase; and a second unit comprising a second dsDNA binding polypeptide associated with a deaminase, wherein the first unit and second unit, when expressed in the individual, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA. In some embodiments, the nucleobase editor system or precursor thereof comprises one or more polynucleotides encoding a double-stranded (ds) DNA binding polypeptide associated with a single-stranded (ss-) nickase and a deaminase, wherein, when expressed in the individual, are configured such that the dsDNA binding polypeptide, when associated with a dsDNA, position the ss-nickase and the deaminase with a site of action for the ss-nickase and a site of action for the deaminase within an editing region on the dsDNA. In some embodiments the disease is a mitochondrial disease. In some embodiments, the mitochondrial disease, including condition, is autism spectrum intellectual disability, 3-methylglutaconic aciduria (3-MGA), Charcot-Marie-Tooth disease, mitochondrial encephalopathy, lactic acidosis and stroke-like episodes (MELAS) syndrome, epilepsy, myoclonic epilepsy, myoclonic epilepsy with ragged red fibers (MERRF), neuropathy, ataxia syndromes, spinocerebellar ataxia, ataxia and retinitis pigmentosa (NARP) syndrome, myotonic dystrophy (DM), Duchenne muscular dystrophy (DMD), Leber hereditary optic neuropathy (LHON), Leber optic atrophy and dystonia (LDYT), Leigh syndrome, Kearns-Sayre syndrome (KSS), Pearson syndrome, chronic progressive external ophthalmoplegia (CPEO), focal segmental glomerulosclerosis (FSGS), Gitelman-like syndrome, mitochondrial myopathy lactic acidosis and sideroblastic anemia (MLASA), lactic acidemia, maternally inherited diabetes and deafness (MIDD), Rhabdomyolysis, non-insulin diabetes mellitus (NIDM), aminoglycoside induced hearing disorders, Alpers' disease, Complex I deficiency, Complex II deficiency, Complex III deficiency, Complex IV deficiency, Complex V deficiency, cardiomyopathy, maternally inherited cardiomyopathy (MICM), hypertrophic cardiomyopathy (HCM), infantile cardiomyopathy, encephalomyopathy, progressive encephalomyopathy, progressive mito cytopathy, deafness, dementia, depressive mood disorder, dystonia, progressive dystonia, exercise intolerance, hyperammonemia, IgG nephropathy, leukoencephalopathy, maternally inherited epilepsy, maternally inherited non-syndromic deafness, mito tubulointerstitial kidney disease (MITKD), mitochondrial myopathy, severe adult-onset multisymptom myopathy, multiple myeloma (MM), myelomeningocele (MMC), mitochondrial neurogastrointestinal encephalopathy (MNGIE) syndrome, optic atrophy, myoclonus, post-exertional malaise (PEM), ptosis, renal insufficiency, reversible COX deficiency myopathy, septo-optic dysplasia, sensorineural hearing loss (SNHL), spastic paraplegia, stroke, or mitochondrial cytopathies. In some embodiments, the individual has Leber hereditary optic neuropathy (LHON). In some embodiments, the mutated DNA is mitochondrial DNA. In some embodiments, the editing region comprises the DNA mutation, e.g., the target nucleotide edited by the nucleobase editor system is (or includes) a nucleotide of the DNA mutation(s). In some embodiments, the editing region comprises another DNA feature associated with the disease, DNA mutation, or a mechanism providing, at least in part, treatment of the disease. For example, in some embodiments, the editing region comprises a start codon, e.g., such that the nucleobase editing system modulates (including inhibits or prohibits) expression of a gene. In some embodiments, the one or more polynucleotides administered to the individual comprise RNA, such as a circular RNA.

In some embodiments, the methods of use provided herein, such as a method of treatment, comprise use of a nucleobase editor system configured for editing two or more nucleotide bases, e.g., nucleotide bases at different DNA locations. In some embodiments, when the nucleobase editor system is configured for editing a plurality of nucleotide bases, at least two or more nucleotide bases of the plurality are in different editing regions. Various configurations of nucleobase editor systems described herein can be used to perform such editing. For example, in some embodiments, the nucleobase editor system configurated for editing a plurality of nucleotide bases comprises a first unit comprising a first double-stranded (ds) DNA binding polypeptide associated with a first single-stranded (ss-) nickase; a second unit comprising a second dsDNA binding polypeptide associated with a second ss-nickase, wherein the first dsDNA binding polypeptide and the second dsDNA binding polypeptide recognize different recognition sequences; a third unit comprising a third dsDNA binding polypeptide associated with a first deaminase; and a fourth unit comprising a fourth dsDNA binding polypeptide associated with a second deaminase, wherein the nucleobase editor system is configured such that the first dsDNA binding polypeptide of the first unit and the third dsDNA binding polypeptide of the third unit, when associated with a dsDNA, position the first ss-nickase and the first deaminase such that a site action for the first ss-nickase and a site of action for the first deaminase are within a first editing region on the dsDNA, and wherein the nucleobase editor system is configured such that the third dsDNA binding polypeptide of the third unit and the fourth dsDNA binding polypeptide of the fourth unit, when associated with the dsDNA, position the second ss-nickase and the second deaminase such that a site action for the second ss-nickase and a site of action for the second deaminase are within a second editing region on the dsDNA. In some embodiments, the first ss-nickase and the second ss-nickase recognize different recognition sequences. In some embodiments, the first deaminase and the second deaminase are the same. In some embodiments, the first deaminase and the second deaminase catalyze the same nucleotide base conversions. In some embodiments, the first deaminase and the second deaminase catalyze different nucleotide base conversions. In some embodiments, the treatment is for Leber hereditary optic neuropathy (LHON).

In some embodiments, provided is a use of a nucleobase editor system described herein in the manufacture of a medicament for treating a disease in an individual, such as a mitochondrial disease.

In some embodiments, the nucleobase editor system has a first editing efficiency (such as measured by an editing percentage) in a first cell type and a second editing efficiency in a second cell type, wherein the first editing efficiency is different than the second editing efficiency. For example, in some embodiments, the nucleobase editor system may be configured to have cell type or tissue specificity, wherein the editing efficiency of the nucleobase editor system is higher in a targeted cell type or tissue and lower or not substantially occurring (such as an editing percentage of about 5% or less) in a different cell type or tissue.

In some embodiments, the efficiency of editing of the target DNA nucleotide base is at least about 10%, such as at least about any one of 15%, 20%, 25%, 30%, 35%, 40%, 45%, 50%, 55%, 60%, or 65%, or higher. In some embodiments, the efficiency of editing is determined by Sanger sequencing. In some embodiments, the efficiency of editing is determined by next-generation sequencing.

In some embodiments, the method has a low off-target editing rate. In some embodiments, the method has lower than about 5% (e.g., no more than about any one of 4.0%, 3.0%, 2.0%, 1.0%, 0.5%, 0.1%, 0.05%, 0.01%, 0.001% or lower) editing efficiency on a non-target DNA nucleotide base as compared to the target DNA nucleotide base. In some embodiments, the method does not edit non-target DNA nucleotide bases.

In certain aspects, provided herein are methods of making and practicing the description provided herein. Unless otherwise stated, the methods of making and practicing can be accomplished using convention teachings of molecular biology, microbiology, and cell biology, which are well known by one of ordinary skill in the art. See, e.g., “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). Such teachings and knowledge of one of ordinary skill in the art are applicable to the production of the polynucleotides and polypeptides described herein. Certain techniques are illustrated in the Examples section provided herein.

### IV. Kits, Medicines, and Compositions

Provided herein, in certain aspects, are kits and compositions of the nucleobase editor systems taught herein, including ss-nickases having desired recognition sequences.

In some embodiments, provided herein is a kit for a nucleobase editing system, the kit comprising: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In some embodiments, provided herein is a kit for a nucleobase editor system, the kit comprising one or more polynucleotides encoding: a first unit comprising a first double-stranded (ds) DNA binding polypeptide associated with a single-stranded (ss-) nickase; and a second unit comprising a second dsDNA binding polypeptide associated with a deaminase, wherein the nucleobase editor system, when expressed, is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

In some embodiments, provided herein is a kit for a nucleobase editor system, the kit comprising one or more polynucleotides encoding: a single-stranded (ss-) nickase; a deaminase; and a double-stranded (ds) DNA binding polypeptide, wherein the ss-nickase, the deaminase, and dsDNA binding polypeptide form a complex configured such that the dsDNA binding polypeptide, when associated with a dsDNA, positions the ss-nickase and the deaminase such that a site action for the ss-nickase and a site of action for the deaminase are within an editing region on the dsDNA.

Kits provided herein may include one or more containers, and instruction for use thereof according to the methods provided herein. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits provided herein are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

Also provided are medicines, compositions, and unit dosage forms useful for the methods described herein.

In certain aspects, provided herein is one or more non-naturally occurring polypeptides forming a nucleobase editor system described herein. In some embodiments, one of the one or more non-naturally occurring polypeptides forming a nucleobase editor system described herein comprises a polypeptide comprising nickase activity and a polypeptide comprising ds-DNA binding, such as a TALE. In some embodiments, one of the one or more non-naturally occurring polypeptides forming a nucleobase editor system described herein comprises a polypeptide comprising deaminase activity and a polypeptide comprising ds-DNA binding, such as a TALE. In some embodiments, one of the one or more non-naturally occurring polypeptides forming a nucleobase editor system described herein comprises a polypeptide comprising nickase activity, a polypeptide comprising deaminase activity, and a polypeptide comprising ds-DNA binding, such as a TALE, e.g., a polypeptide comprising a sequence of SEQ ID NOs:21-24.

In certain aspects, provided herein is a non-naturally occurring polypeptide having nickase activity, the non-naturally occurring polypeptide comprising the amino acid sequence of SEQ ID NO:1 with the following mutations of E91A and F94A (SEQ ID NO:2). In certain aspects, provided herein is a non-naturally occurring polypeptide having nickase activity, the non-naturally occurring polypeptide comprising the amino acid sequence of SEQ ID NO:2 (sometimes referred to herein as MutH*). In some embodiments, the non-naturally occurring polypeptide is isolated. In some embodiments, provided herein is a polynucleotide encoding the non-naturally occurring polypeptide comprising the amino acid sequence of SEQ ID NO:2. In some embodiments, the non-naturally occurring polypeptide having nickase activity and comprising the amino acid sequence of SEQ ID NO:2, recognizes the ss-nickase recognition sequence of 5′-GATD-3′, D is for A, T or G.

### V. Sequences

Provided herein, in certain aspects, are sequences useful for the nucleotide editor systems provided herein. Specifically, ss-nickase sequences are provided for MutH and a derivative thereof, MutH*, having the following mutations of E91A and F94A. MutH* has a ss-nickase recognition sequence of 5′-GATD-3′, D is for A, T or G.

MutH (Escherichia coli); protein sequence;
SEQ ID NO: 1
MSQPRPLLSPPETEEQLLAQAQQLSGYTLGELAALVGLVTPENLKRDKGWIGVLLEIWL
GASAGSKPEQDFAALGVELKTIPVDSLGRPLETTFVCVAPLTGNSGVTWETSHVRHKLK
RVLWIPVEGERSIPLAQRRVGSPLLWSPNEEEDRQLREDWEELMDMIVLGQVERITARH
GEYLQIRPKAANAKALTEAIGARGERILTLPRGFYLKKNFTSALLARHFLIQ
MutH*; protein sequence;
SEQ ID NO: 2
MSQPRPLLSPPETEEQLLAQAQQLSGYTLGELAALVGLVTPENLKRDKGWIGVLLEIWL
GASAGSKPEQDFAALGVELKTIPVDSLGRPLATTAVCVAPLTGNSGVTWETSHVRHKL
KRVLWIPVEGERSIPLAQRRVGSPLLWSPNEEEDRQLREDWEELMDMIVLGQVERITAR
HGEYLQIRPKAANAKALTEAIGARGERILTLPRGFYLKKNFTSALLARHFLIQ
Nt.BspD6I(C); protein sequence;
SEQ ID NO: 3
RQLEEVIDLLEVYHEKKNVIEEKIKARFIANKNTVFEWLTWNGFIILGNALEYKNNFVID
EELQPVTHAAGNQPDMEIIYEDFIVLGEVTTSKGATQFKMESEPVTRHYLNKKKELEKQ
GVEKELYCLFIAPEINKNTFEEFMKYNIVQNTRIIPLSLKQFNMLLMVQKKLIEKGRRLSS
YDIKNLMVSLYRTTIECERKYTQIKAGLEETLNNWVVDKEVRF
FokI-FokI(D450A); protein sequence;
SEQ ID NO: 4
FKQLVKSELEEKKSELRHKLKYVPHEYIELIEIARNSTQDRILEMKVMEFFMKVYGYRG
KHLGGSRKPDGAIYTVGSPIDYGVIVDTKAYSGGYNLPIGQADEMQRYVEENQTRNKHI
NPNEWWKVYPSSVTEFKFLFVSGHFKGNYKAQLTRLNHITNCNGAVLSVEELLIGGEMI
KAGTLTLEEVRRKFNNGEINFSSGGGGSGGGGSGGGGSGGSGGGSSGGGGSGGGGSGG
GGSQLVKSELEEKKSELRHKLKYVPHEYIELIEIARNSTQDRILEMKVMEFFMKVYGYR
GKHLGGSRKPAGAIYTVGSPIDYGVIVDTKAYSGGYNLPIGQADEMQRYVEENQTRNK
HINPNEWWKVYPSSVTEFKFLFVSGHFKGNYKAQLTRLNHITNCNGAVLSVEELLIGGE
MIKAGTLTLEEVRRKENNGEINF
Nb.BsaI(C, N441D/R442G); protein sequence;
SEQ ID NO: 5
CRSHADRGRWEKNLRSYTTDRRAFEYWVDGDWVAADKLMGLIRTNEQIKKETCLND
NHPGPCSADHIGPISLGFVHRPEFQLLCNSCNSAKNNRMTFSDVQHLINAENNGEEVAS
WYCKHIWDLRKHDVKNNENALRLSKILRDNRHTAMFILSELLKDNHYLFLSTFLGLQY
AERSVSFSNIKIENHIITGQISEQPRDTKYTEEQKARRMRIGFEALKSYIEKEDGNALLVIN
DKIIDKINEIKNILQDIPDEYKLLNEKISEQFNSEEVSDELLRDLVTHLPTKESEPANFKLA
RKYLQEIMEIVGDELSKMWEDERYVRQTFADLD
Nt.BsaI(C, R236D); protein sequence;
SEQ ID NO: 6
CRSHADRGRWEKNLRSYTTDRDAFEYWVDGDWVAADKLMGLIRTNEQIKKETCLND
NHPGPCSADHIGPISLGFVHRPEFQLLCNSCNSAKNNRMTFSDVQHLINAENNGEEVAS
WYCKHIWDLRKHDVKNNENALRLSKILRDNRHTAMFILSELLKDNHYLFLSTFLGLQY
AERSVSFSNIKIENHIITGQISEQPRDTKYTEEQKARRMRIGFEALKSYIEKENRNALLVIN
DKIIDKINEIKNILQDIPDEYKLLNEKISEQFNSEEVSDELLRDLVTHLPTKESEPANFKLA
RKYLQEIMEIVGDELSKMWEDERYVRQTFADLD
Nb.BsmBI(C, R438D); protein sequence;
SEQ ID NO: 7
KDPGRHDDNMRLYNHDRRAFMWWSEGDWALADALYNKAGAGKCADPDCQKEVEKI
SPDHVGPISCGFKQIPFFKPLCASCNSAKNRRFSYQDVKELLKYENYTGDSVASWQVRA
LWDNCKHLVKNDDDSKLLSNLMRSLQDYYLRSLYKLFSNGFAHLLSYFLTPEYAHYKI
TFEGLNTSTLEYERYYKTFKKTKSTSSLAARIVRIAFEELEIYNSKDINEDKLIKFDTSSWE
KDFENIISYATKNLSLDEEASKWNKVLTDKNLSSTEKDKKISSLLEDKNYEVYKKQFYIL
KDLLVEHFNKIGEQIAKDYMK
Nt.BsmAI(C, R221D); protein sequence;
SEQ ID NO: 8
CRASQDKGRSKENLKSYTKDRDAYEYWSDGNIHAANQFMGSPFFNNISADHIGPISLGF
VHDPRYLQPMSGGDNSSKRDRLQLDDIEKIIETEKRTNVYPMSWYSKLIWEYIKKNYST
HKSLISGVYRDALKQNMSNFMYILWYILEHCNQDGEHFLEEALLKPNYDYFQYSYTFN
ELGEIVSINPRHFTDRNQYETERYKRIAFESVYDYNEKENRNIKANLIDNEQRMLNKLCQ
EISSGVPVEQCKKLLIELMEVIQKRIISTL
Nb.BsrDI(C); protein sequence;
SEQ ID NO: 9
IPEELFNWPRTDKVNFKSPQGLIKYDELCYQLEKAVGSKKAYCLSNNAGAKPQKLESLK
EWINSQKKLFDKAPKLTPPAEFNMKLDAFPVTSNNNYYVTTSKNILYLFDYWKDLRIAI
ETAFPRLKGKLPTDIDEKPALIYICNSVKPGRLFGDPFTGQLSAFSTIFGKKNIDMPRIVVA
YYPHQIYSQALPKNNKSNKGITLKKELTDFLIFHGGVVVKLNEGKAY
Nt.CviPII; protein sequence;
SEQ ID NO: 10
MYIYMSTPQAKTKYYEQRFVNDFYKELERNKVSLPVTIVLKDNLGIKQVIQNGSGVRVL
RDKANAKSPSKIKSEELGRHVTSKADIALFTEEKNGTKVDVAWISPQSHKDFLGKKITPA
QYFDASSDVMFKTKIGQPKEIKELKNKMISLSVPLTATKYCWPKYKSGTSLRIWDDVQS
TILMNMAIFGVEFGKAYCRNNANILMVGDPLIEVKDDKTIILTTKENGFSLANGFAEYIP
SKDKPIFFTKPTSGKKTVVDGKTIEGVSVWIIYRSYAGSKNRKIDDVLKNKIELISSSCSV
KKKDNFVSIMQSKKITSPPKSKKITSPPKSKKITSPSKSKKITNFFMKK
BspQI(C); protein sequence;
SEQ ID NO: 11
NNSFNPVRTKDQLHESAVITREKKILLKEPEILQKIKNRNNGEGLKSIIWKKFDKKCFNC
EKELTIEEVRLDHTRPLAYLWPIDEHATCLCEKCNNTKHDMFPIDFYQGDEDKLRRLAR
ITGLDYESLVKRDVNEVELARIINNIEDFATNVEARTFRSIRNKVKEVRPDTDLFEILKSK
NINLYNELQYELLTRKD
N.AlwI(C); protein sequence;
SEQ ID NO: 12
YHLEELLFENNEKKFAENQKNEWDEILAYMDLLISPKPISIEIADKEISIPSGERPAYFEW
VLWRAFLALNHLIIEPQQCRRFKVDQDFKPIHNAPGGGADVIFEYENFKILGEVTTSKGA
TQFKMESEPVTRHYLNKKKELEKQGVEKELYCLFIAPEINKNTFEEFMKYNIVQNTRIIP
LSLKQFNMLLMVQKKLIEKGRRLSSYDIKNLMVSLYRTTIECERKYTQIKAGLEETLNN
WVVDKEVRF
Nt.BsrDI; protein sequence;
SEQ ID NO: 13
MTEYDLHLYADSFHEGHWCCENLAKIAQSDGGKHQIDYLQGFIPRHSLIFSDLIINITVFG
SYKSWKHLPKQIKDLLFWGKPDFIAYDPKNDKILFAVEETGAVPTGNQALQRCERIYGS
ARKQIPFWYLLSEFGQHKDGGTRRDSIWPTIMGLKLTQLVKTPSIILHYSDINNPEDYNS
GNGLKFLFKSLLQIIINYCTLKNPLKGMLELLSIQYENMLEFIKSQWKEQIDFLPGEEILNT
KTKELARMYASLAIGQTVKIPEELFNWPRTDKVNFKSPQGLIKYDELCYQLEKAVGSKK
AYCLSNNAGAKPQKLESLKEWINSQKKLFDKAPKLTPPAEFNMKLDAFPVTSNNNYYV
TTSKNILYLFDYWKDLRIAIETAFPRLKGKLPTDIDEKPALIYICNSVKPGRLFGDPFTGQ
LSAFSTIFGKKNIDMPRIVVAYYPHQIYSQALPKNNKSNKGITLKKELTDFLIFHGGVVV
KLNEGKAY
Nt.BtsI; protein sequence;
SEQ ID NO: 14
MQIEQLMKSLTIYFDDIQEGLWFKNLHPLLESASLEAITGSLKRNPNLADVLKYDRPDIIL
TLNQTPILVIERTIEVPSGHNVGQRYGRLAAASEAGVPLVYFGPYAARKHGGATEGPRY
MNLRLFYALDVMQKVNGSAITTINWPVDQNFEILQDPSKDKRMKEYLEMFFDNLLKYG
IAGINLAIRNSSFQAEQLAEREKFVETMITNPEQYDVPPDSVQILNAERFFNELGISENKRI
ICDEVVLYQVGMTYVRSDPYTGMALLYKYLYILGSERNRCLILKFPNITTDMWKKVAF
GSRERKDVRIYRSVSDGILFADGYLSKEEL
I-TevI; protein sequence;
SEQ ID NO: 15
KSGIYQIKNTLNNKVYVGSAKDFEKRWKRHFKDLEKGCHSSIKLQRSFNKHGNVFECSI
LEEIPYEKDLIIERENFWIKELNSKINGYNIADATFGDTCSTHPLKEEIIKKRSETVKAKML
KLGPDGRKALYSKPGSKNGRWNPETHKFCKCGVRIQTSAYTCSKCRNRSGENNSFFNH
KHSQGPSAD
ss.BspD6I; protein sequence;
SEQ ID NO: 16
MQDILDFYEEVEKTINPPNYFEWNTYRVFKKLGSYKNLVPNFKLDDSGHPIGNAIPGVE
DILVEYEHFSILIECSLTIGEKQLDYEGDSVVRHLQEYKKKGIEAYTLFLGKSIDLSFARHI
GFNKESEPVIPLTVDQFKKLVTQLKGDGEHENPNKLKEILIKLLRSDLGYDQAEEWLTFI
EYNLK
ss.BsrDI; protein sequence;
SEQ ID NO: 17
MTDYRYSFELSEEIARWAFEIKTKNTDWFVAFSNPTAGPWKRVMAIDKASNREGEVHR
FGREDERPDIILVNDNISLILILEAKEKLNQLISKSQVDKSVDVFLTLSSILKEKSDNNYWG
DRTKYINVLGILWGSEQETSQKDIDNAFRVYRDSLVKNLKEINPTPTNICTDILVGVESIK
NKKEEISIKIHVSNIYAEIYPKFTGKHLLEKLAVLN
ss.BtsI; protein sequence;
SEQ ID NO: 18
MKITEGIVHVAMRHFLKSNGWKLIAGQYPGGSDDELTALNIVDPVVARDNSPDPRRHS
LGKIVPDLIAYKNDDLLVIEAKPKYSQDDRDKLLYLLSERKHDFYAALEKFATERNHPE
LLPVSKLNIIPGLAFSASENKFKKDPGFVYIRVSGIFEAFMEGYDWG
TadA8e(V106W); protein sequence;
SEQ ID NO: 19
SEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGWRNSKRGAAG
SLMNVLNYPGMNHRVEITEGILADECAALLCDFYRMPRQVFNAQKKAQSSIN
rAPOBEC1-2×UGI; protein sequence;
SEQ ID NO: 20
SSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHA
DPRNRQGLRDLISSGVTIQIMTEQESGYCWRNFVNYSPSNEAHWPRYPHLWVRLYVLE
LYCIILGLPPCLNILRRKQPQLTFFTIALQSCHYQRLPPHILWATGLKGSGGSTNLSDIIEK
ETGKQLVIQESILMLPEEVEEVIGNKPESDILVHTAYDESTDENVMLLTSDAPEYKPWAL
VIQDSNGENKIKMLSGGSGGSGGSTNLSDIIEKETGKQLVIQESILMLPEEVEEVIGNKPE
SDILVHTAYDESTDENVMLLTSDAPEYKPWALVIQDSNGENKIKML
MutH-2AA linker-TadA8e(V106W); protein sequence;
SEQ ID NO: 21
MSQPRPLLSPPETEEQLLAQAQQLSGYTLGELAALVGLVTPENLKRDKGWIGVLLEIWL
GASAGSKPEQDFAALGVELKTIPVDSLGRPLETTFVCVAPLTGNSGVTWETSHVRHKLK
RVLWIPVEGERSIPLAQRRVGSPLLWSPNEEEDRQLREDWEELMDMIVLGQVERITARH
GEYLQIRPKAANAKALTEAIGARGERILTLPRGFYLKKNFTSALLARHFLIQG*S*SEVEF
SHEYWMRHALTLAKRARDEREVPVGAVLVLNNRVIGEGWNRAIGLHDPTAHAEIMAL
RQGGLVMQNYRLIDATLYVTFEPCVMCAGAMIHSRIGRVVFGWRNSKRGAAGSLMNV
LNYPGMNHRVEITEGILADECAALLCDFYRMPRQVFNAQKKAQSSIN
*Amino acids (AA) of linker denoted with a trailing asterisk.
TadA8e(V106W)-2AA linker- MutH; protein sequence;
SEQ ID NO: 22
SEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGWRNSKRGAAG
SLMNVLNYPGMNHRVEITEGILADECAALLCDFYRMPRQVFNAQKKAQSSING*S*MS
QPRPLLSPPETEEQLLAQAQQLSGYTLGELAALVGLVTPENLKRDKGWIGVLLEIWLGA
SAGSKPEQDFAALGVELKTIPVDSLGRPLETTFVCVAPLTGNSGVTWETSHVRHKLKRV
LWIPVEGERSIPLAQRRVGSPLLWSPNEEEDRQLREDWEELMDMIVLGQVERITARHGE
YLQIRPKAANAKALTEAIGARGERILTLPRGFYLKKNFTSALLARHFLIQ
*Amino acids of linker denoted with a trailing asterisk.
Nt.BspD6I(C)-2AA linker-TadA8e(V106W); protein sequence;
SEQ ID NO: 23
RQLEEVIDLLEVYHEKKNVIEEKIKARFIANKNTVFEWLTWNGFIILGNALEYKNNFVID
EELQPVTHAAGNQPDMEIIYEDFIVLGEVTTSKGATQFKMESEPVTRHYLNKKKELEKQ
GVEKELYCLFIAPEINKNTFEEFMKYNIVQNTRIIPLSLKQFNMLLMVQKKLIEKGRRLSS
YDIKNLMVSLYRTTIECERKYTQIKAGLEETLNNWVVDKEVRFG*S*SEVEFSHEYWMR
HALTLAKRARDEREVPVGAVLVLNNRVIGEGWNRAIGLHDPTAHAEIMALRQGGLVM
QNYRLIDATLYVTFEPCVMCAGAMIHSRIGRVVFGWRNSKRGAAGSLMNVLNYPGMN
HRVEITEGILADECAALLCDFYRMPRQVFNAQKKAQSSIN
*Amino acids of linker denoted with a trailing asterisk.
TadA8e(V106W)-2AA linker-Nt.BspD6I(C); protein sequence;
SEQ ID NO: 24
SEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGWRNSKRGAAG
SLMNVLNYPGMNHRVEITEGILADECAALLCDFYRMPRQVFNAQKKAQSSING*S*RQL
EEVIDLLEVYHEKKNVIEEKIKARFIANKNTVFEWLTWNGFIILGNALEYKNNFVIDEEL
QPVTHAAGNQPDMEIIYEDFIVLGEVTTSKGATQFKMESEPVTRHYLNKKKELEKQGVE
KELYCLFIAPEINKNTFEEFMKYNIVQNTRIIPLSLKQFNMLLMVQKKLIEKGRRLSSYDI
KNLMVSLYRTTIECERKYTQIKAGLEETLNNWVVDKEVRF
*Amino acids of linker denoted with a trailing asterisk.
3*EAAAK linker; protein sequence;
SEQ ID NO: 25
AEAAAKEAAAKEAAAKEAAAKA
32AA linker; protein sequence;
SEQ ID NO: 26
GSGSSGGSSGSETPGTSESATPESSGGSSGGS

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1: Editing of Mitochondrial DNA by a mitoABE System

This example demonstrates effective mitochondrial DNA editing by a mitoABE nucleobase editor system. Moreover, this example outlines the mechanistic requirements for effective A-to-G mitochondrial base editing.

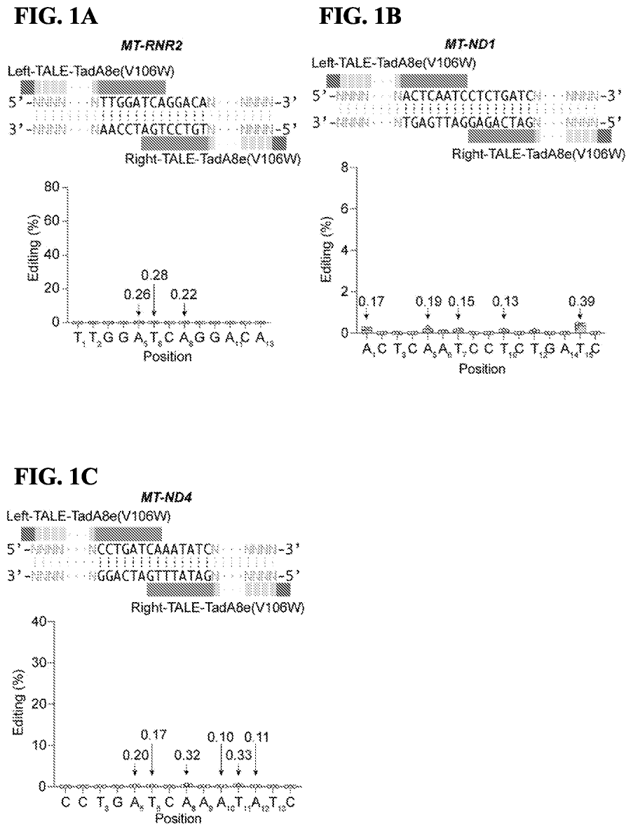

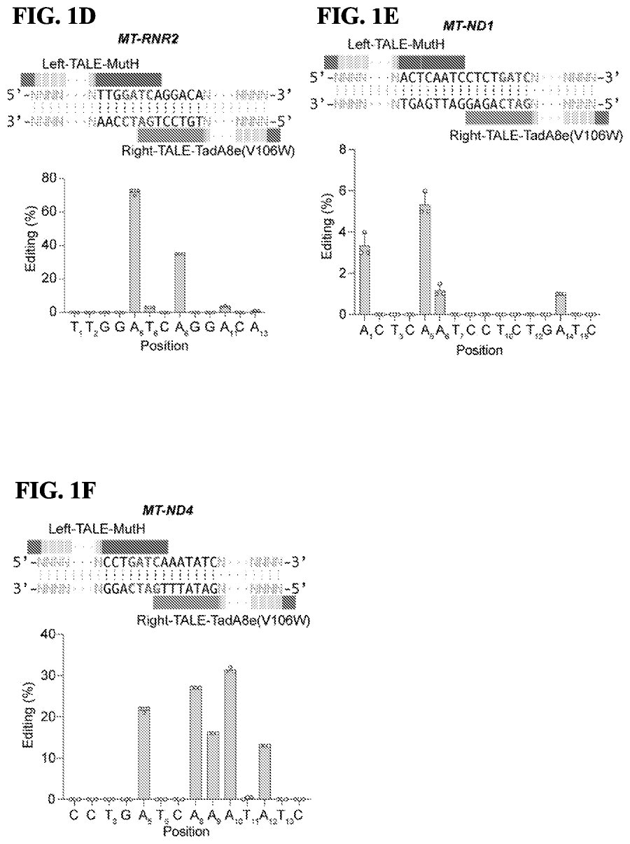

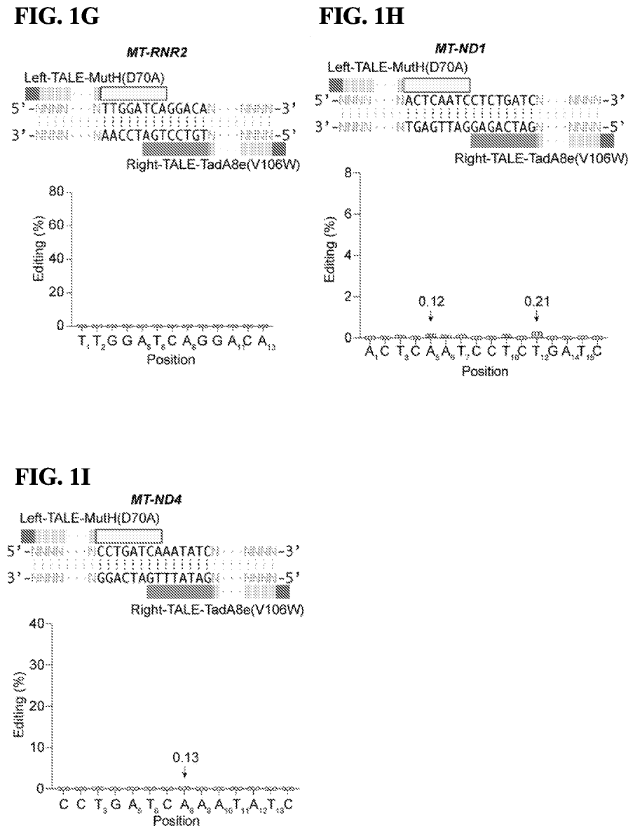

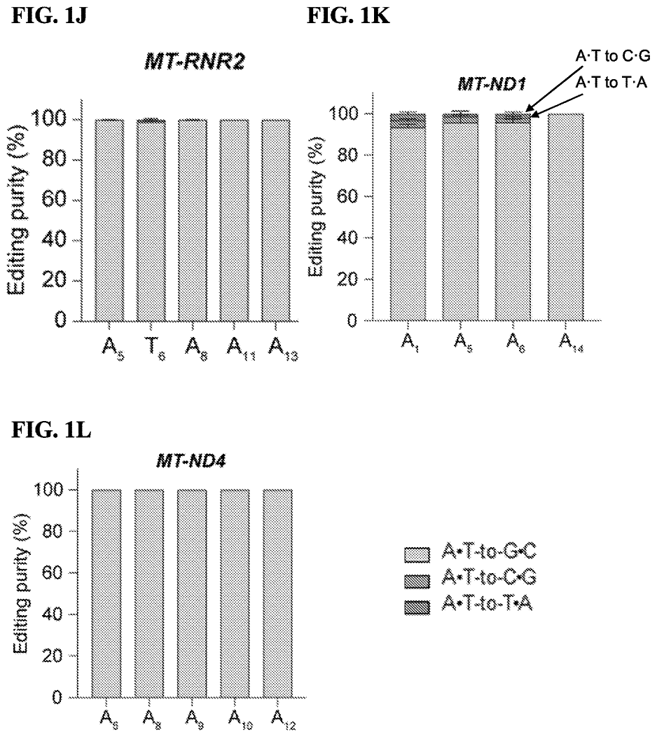

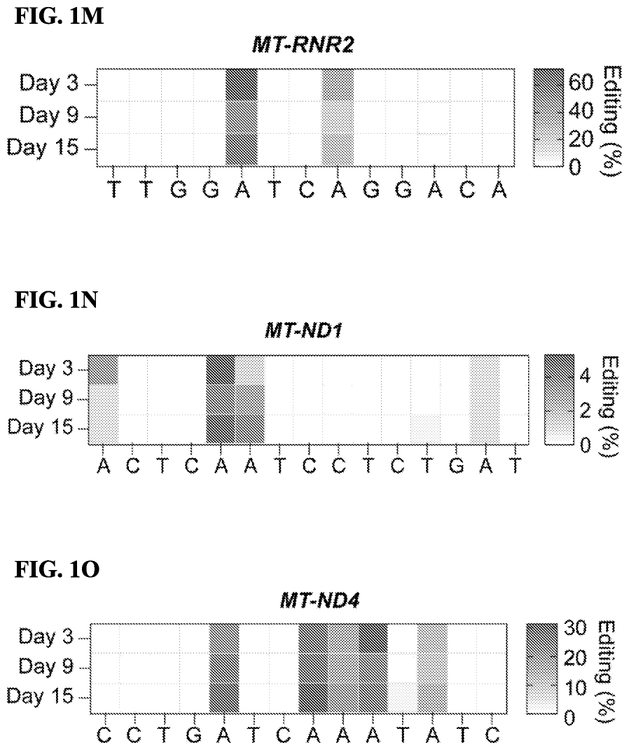

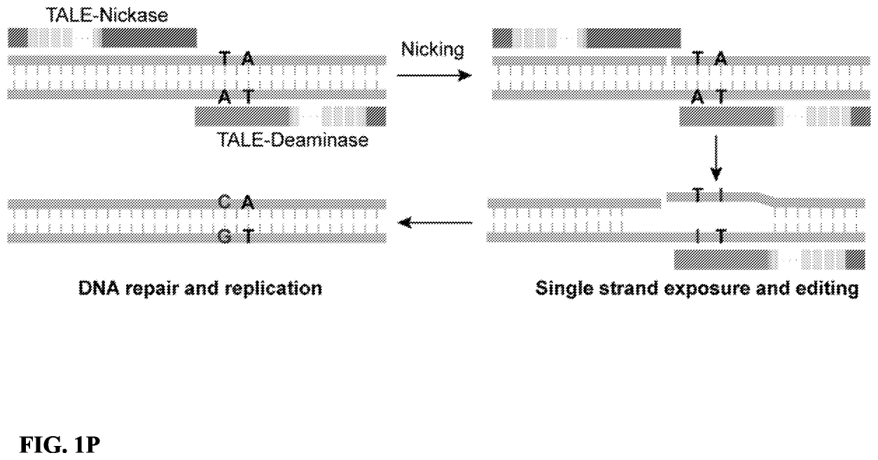

In this example, the engineered deoxyadenosine deaminase, TadA8e(V106W), was fused with an appropriate TALE array and mitochondria targeting sequence (MTS) to target the transcript to the mitochondrial matrix and achieve mitochondrial A-to-G base editing. Mitochondrial DNA editing efficiencies of up to 0.39% were detected in transfected HEK593T cells at all three targeting sites, MT-ND1, MT-ND4, and MT-RNR2 (FIGS. 1A-1C). These low editing efficiencies were barely above the reported deep sequencing error of >0.10%. TadA is an essential tRNA-specific adenosine deaminase originating from Escherichia coli which acts on single-stranded DNA. Because the TALE array is only capable of binding double-stranded DNA and cannot unravel the DNA double helix, a mechanism to induce single-stranded DNA structure at the target site was required to allow TadA8e(V106W) mediated mitochondrial DNA editing.

To induce single-stranded DNA structure at the target site, the sequence-specific (5′-GATC-3′) nickase, MutH, was fused to an appropriate TALE array. The introduction of TALE-MutH and TALE-TadA8e(V106W) constructs in HEK293T cells resulted in targeted A-to-G editing of mitochondrial DNA in all three tested loci (MT-ND1, MT-ND4, and MT-RNR2) with a maximum editing efficiency of 77% (FIGS. 1D-1F). As shown in FIGS. 1G-1I, the catalytically inactive TALE-MutH(D70A), when expressed with TALE-TadA8e(V106W), was unable to effectively edit mitochondrial DNA in transfected HEK293T cells. These results demonstrate that TALE-nickase activity resulted in a single strand nick, exposing single-stranded DNA and generated a substrate for the deoxyadenosine deaminase activity of TadA8e(V106W). This novel mitochondrial A-to-G base editing system will be referred to as mitoABE^{MutH}. The editing purity observed at the three loci tested was greater than 95% at the MT-ND1 locus and close to 100% at the MT-ND4 and MT-RNR2 loci (FIGS. 1J-1L). The durability of mitochondrial DNA editing was confirmed in HEK293T cells at 3, 9, and 15 days at three target loci (MT-ND1, MT-ND4, and MT-RNR2), with mitochondrial DNA editing % consistent throughout the tested timepoints (FIGS. 1M-O).

Based on the above results, a working model was generated whereby the activity of TALE-nickase generates a single strand break at the target site when it binds to mitochondrial DNA. The resulting DNA nick then induces the formation of single-stranded DNA, and TALE-TadA8e(V106W) deaminates the bases on the single-stranded DNA. The editing is then retained only in one strand after repair and mitochondrial DNA replication, resulting in strand-specific A-to-G conversion (FIG. 1P).

### Example 2: Strand-Specific Editing by a mitoABE System

This example demonstrates the strand specific nature of A-to-G editing using a mitoABE system. Strand specificity is established by the strand that is nicked by the TALE-nickase, and adenine deamination is retained only on the non-nicked strand.

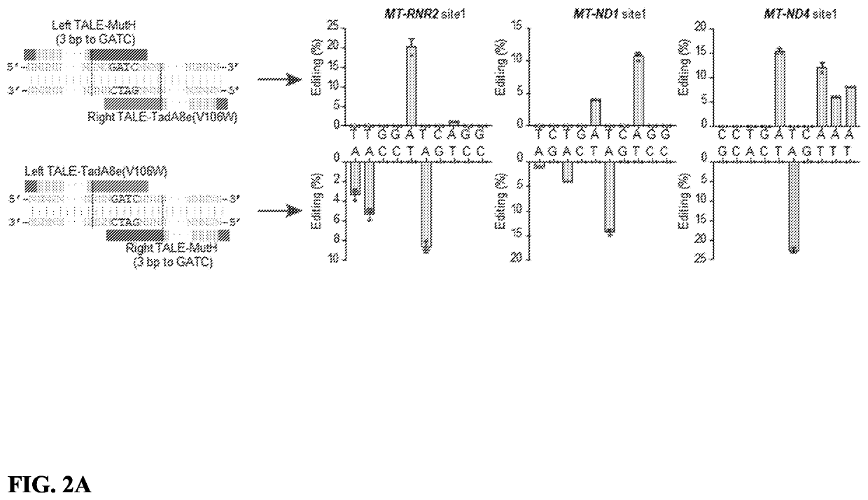

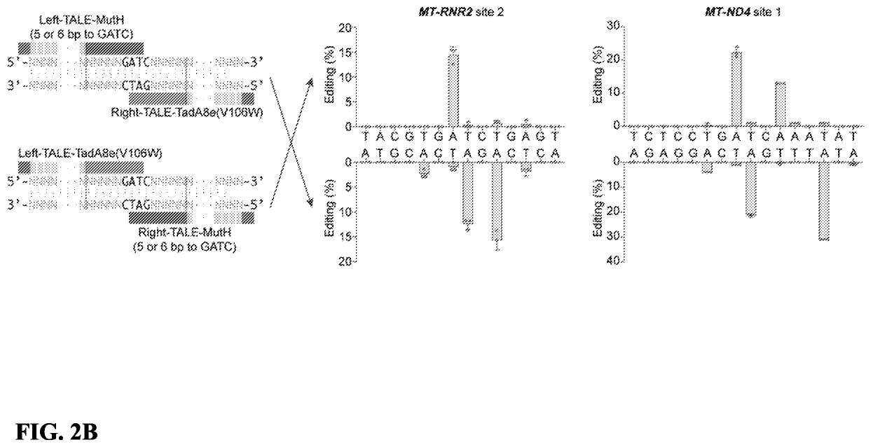

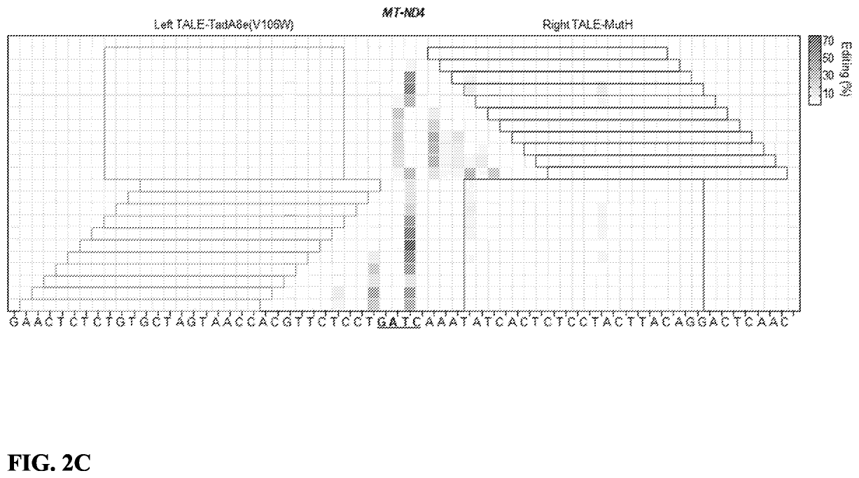

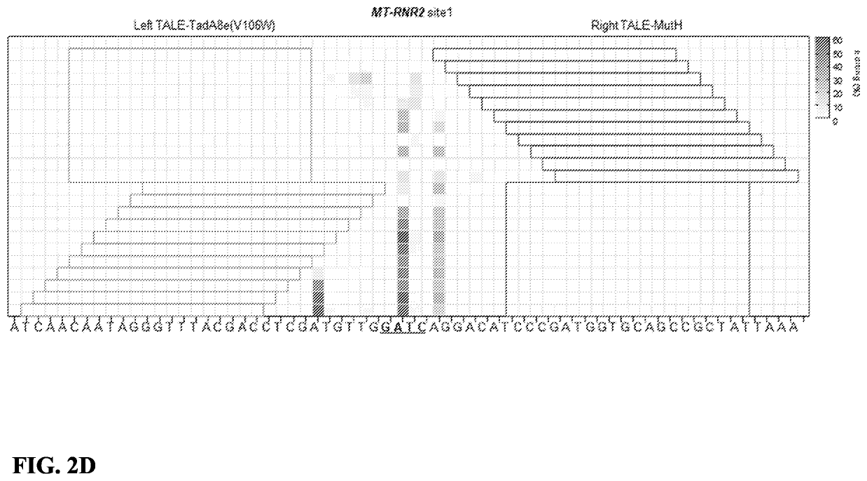

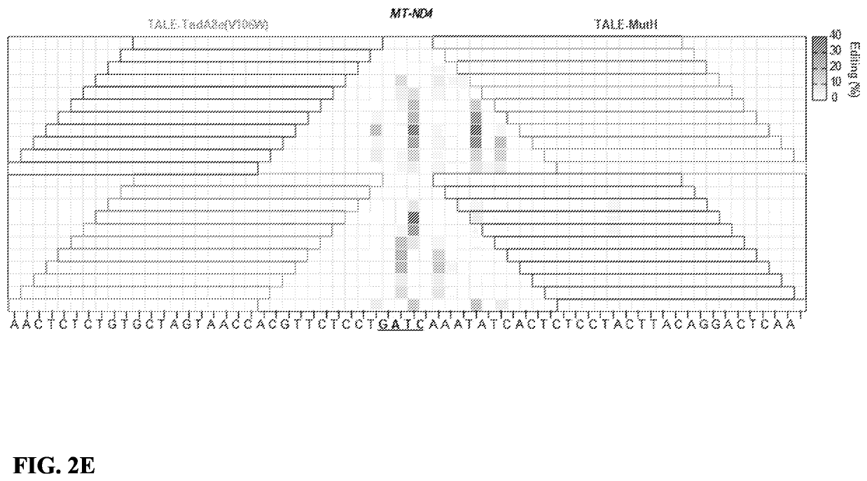

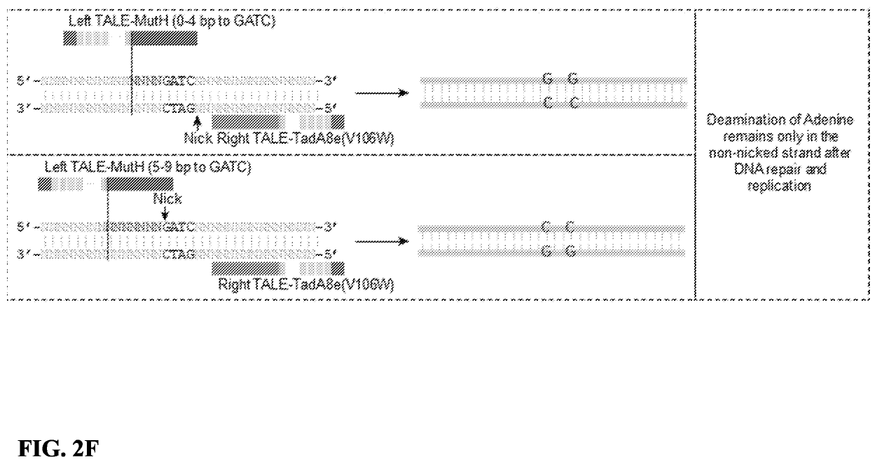

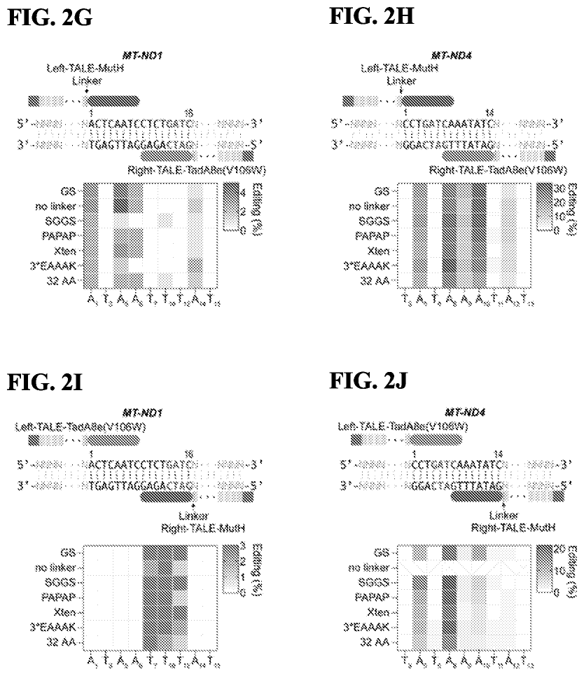

As shown in FIGS. 1D-1F, mitoABE^{MutH}-enabled adenine editing occurred preferentially on the top strands of the above target loci. Despite the presence of multiple adjacent Ts next to the edited A within the editing windows, the A on its opposite strand was not detectibly edited (FIGS. 1E-1F) or was only barely edited (FIG. 1D). These results suggest that the editing of mitoABE^{MutH} may be strand specific. To verify this notion, the TALE arrays were switched for every pair of TALE-MutH and TALE-TadA (V106W) and results demonstrated that the strand-specific edits were reciprocally switched (FIGS. 2A-2B). At MT-RNR2 site 1, MT-ND1 site 1, and MT-ND4 site 1, the MutH nicking sequence (5′-GATC-3′) was placed in the center of the editing window, 3 bp away from each TALE (FIG. 2A). In this case, editing occurred mainly on the top DNA strand using Left-TALE-MutH and Right-TALE-TadA8e(V106W), while editing occurred mainly on the bottom strand when the positions of TALE-MutH and TALE-TadA8e(V106W) were switched (FIG. 2A). At MT-RNR2 site 2 and MT-ND4 site 1, the MutH nicking sequence is 5 or 6 bp away from each end of the editing window (FIG. 2B), and editing occurs mainly on the bottom DNA strand with Left-TALE-MutH and Right-TALE-TadA8e(V106W). The top DNA strand became the A-edited strand after TALEs for MutH and TadA8e(V106W) were switched (FIG. 2B). Strand-specific editing is therefore related to the strand selection of nicking, which in turn is related to the number of bases between TALE binding and the MutH recognition motif (5′-GATC-3′).

Next, the position of Left-TALE-TadA8e(V106W) was fixed and the position of Right-TALE-MutH was shifted from 0 to 10 bp away from the nicking sequence (5′-GATC-3′), successively by 1 bp (FIG. 2C). The editing occurred mainly on the bottom DNA strand at both MT-ND4 and MT-RNR2 site 1 when the distance of the TALE binding and MutH nick motif was within 0 to 4 bp, and the editing occurred mainly on the top DNA strand when the distance of the TALE binding and MutH nick motif was between 5 to 9 bp (FIGS. 2C-2D). In contrast, when the TALE-MutH was fixed and the position of TALE-TadA8e(V106W) was changed, the A-edited strand did not change, but the observed editing windows gradually widened (FIG. 2C).

Since the MutH nick motif (5′-GATC-3′) is a palindromic sequence, it was hypothesized that fusing MutH with the Left-TALE or the Right-TALE should have the same effect. By gradually widening the editing window at the MT-ND4 site (FIG. 2E), results showed that the edited strand switched regardless of whether TALE-MutH was placed to the left or the right of the targeting site. At this site, editing occurred efficiently between the 6- and 24-bp editing windows (FIG. 2E). These results underscore the basic working principle of mitoABE^{MutH}, in which TALE-MutH nicks the opposite strand from its binding strand when the distance of TALE binding and MutH nick motif was between 0-4 bp, and it nicks the same strand when the distance of the TALE binding and the MutH nick motif was between 5-9 bp. Nicking generated two single-stranded DNAs, and all adenines on both strands were subjected to TadA8e(V106W)-mediated deamination within the window. After repair and DNA replication, only deaminated As on the non-nicked strand were retained (FIG. 2F). Collectively, this data demonstrated that the editing outcome of TadA8e(V106W) depends on which strands are nicked by the TALE-nickase, and adenine deamination is retained only on the non-nicked strand. In addition, the linker sequences between the TALE and MutH had no effect on the strand-specific editing of the mitoABE^{MutH} system (FIGS. 2G-J).

### Example 3: Expanding the Targeting Scope of a mitoABE^{MutH} System by Site-Directed Mutations

This example demonstrates that the sequence specificity of nickases can be broadened by targeted mutations. Moreover, this approach can be utilized to improve editing efficiency in the mitoABE system.

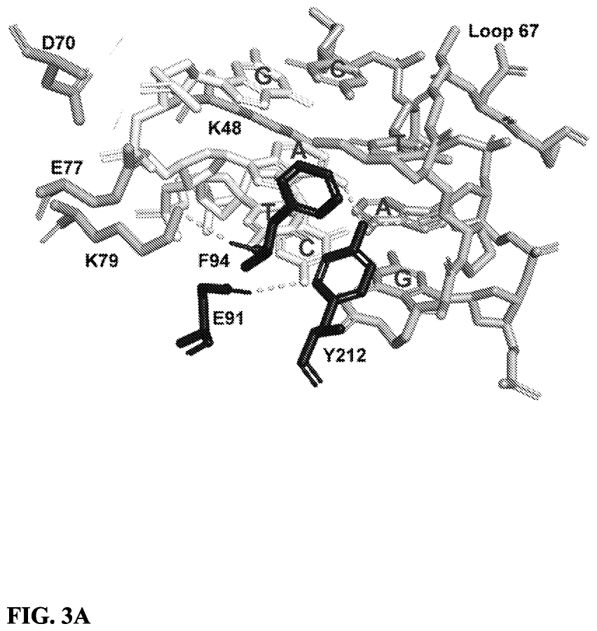

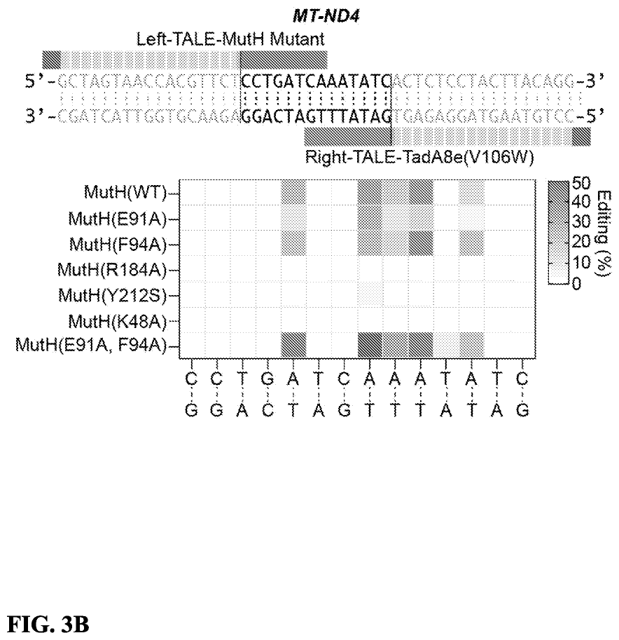

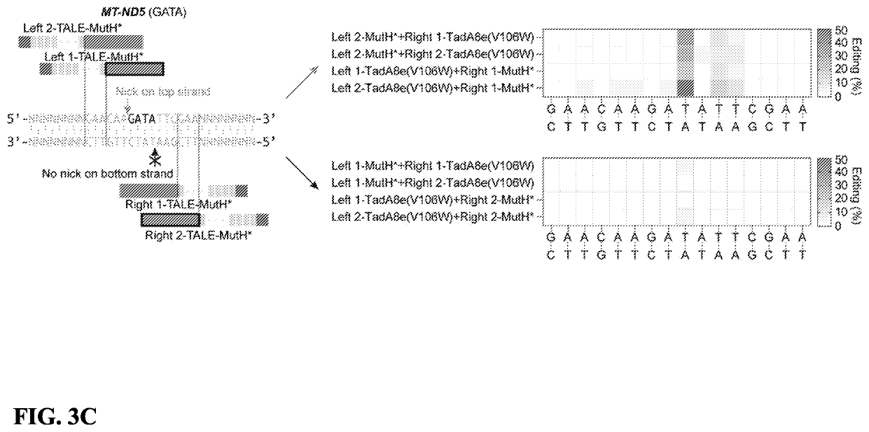

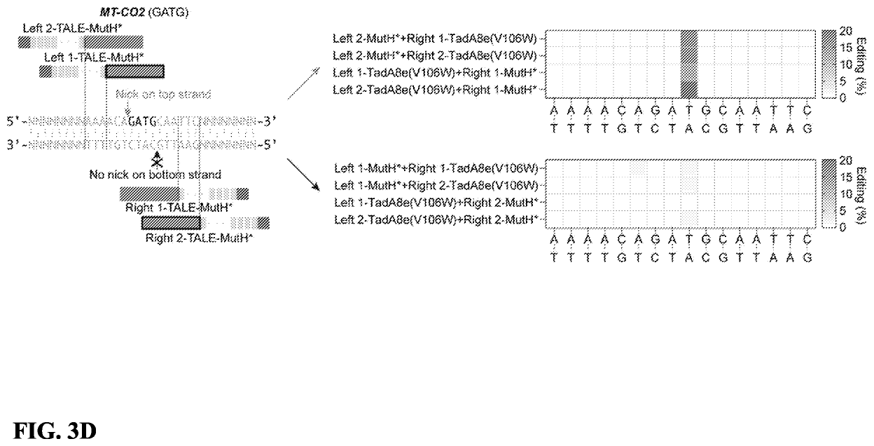

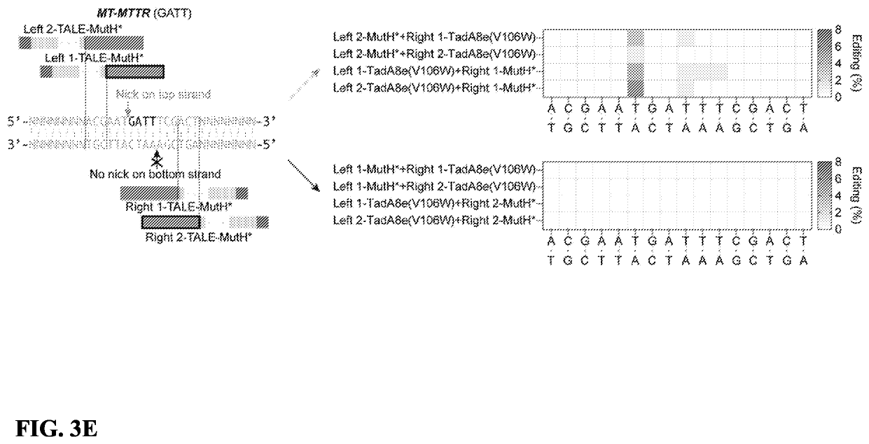

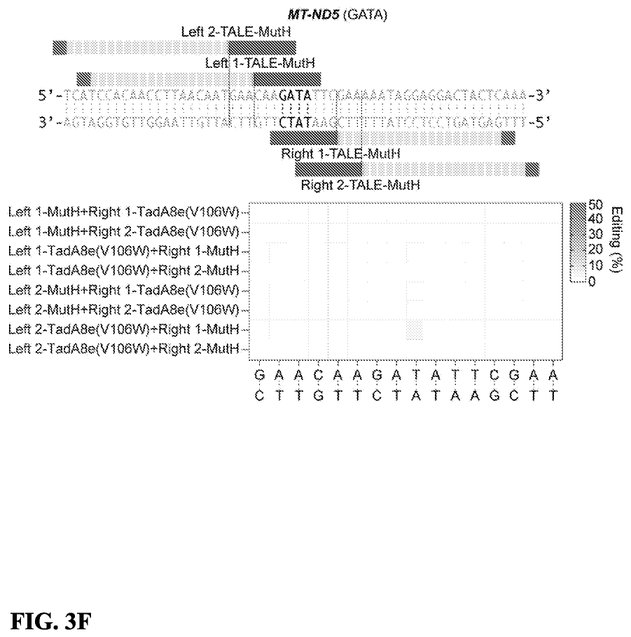

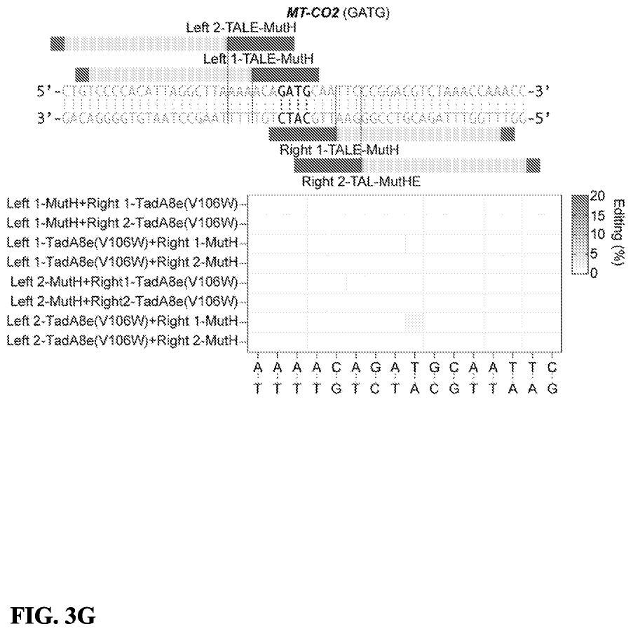

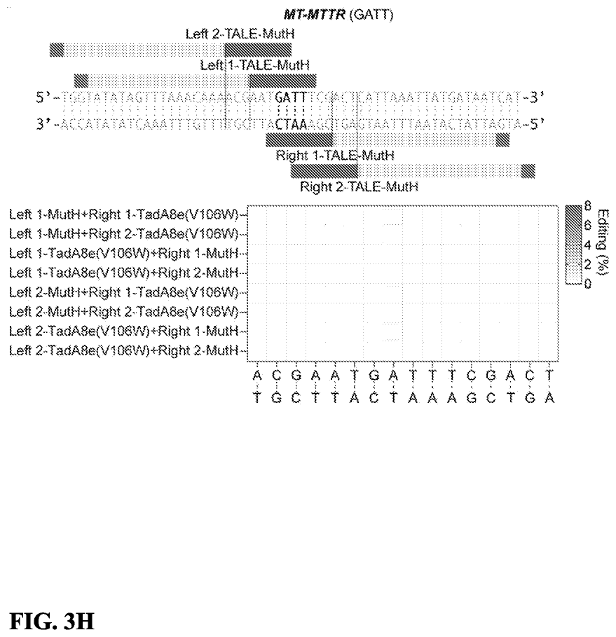

The combination of TALE-MutH and TALE-TadA(V106W) enables targeted strand-specific editing of mitochondrial DNA. However, MutH requires a specific sequence (5′-↓ GATC-3′) for nicking, limiting the editing scope of mitoABE^{MutH}. Based on structural information, the editing scope of mitoABE^{MutH} was expanded by introducing point mutation(s) to MutH. Results demonstrated that K48A, R184A, and Y212S mutations abolished the editing activity of mitoABE^{MutH}, at the MT-ND4 site (FIG. 3A, B). In MutH, F94 helps loop 67 (residues 184-190 AA) to make sequence-specific interactions with 5′-GATC-3′, and F91 interacts with the cytosine in 5′-GATC-3′ (FIG. 3A). The E91A or F94A variant maintained the editing activity of mitoABE^{MutH}, and the combination of these two mutations enhanced the editing efficiency at the ND4 site (FIG. 3B). This MutH enzyme harboring E91A and F94A mutations will hereby be designated as MutH*. As shown in FIG. 3B, MutH* could generate nicks at 5′-GATD-3′ (D stands for A, T or G) sites and become a new type of mtDNA editing tool, mitoABE^{MutH*}. By targeting three loci, MT-ND5, MT-CO2, and MT-MTTR, which, respectively contain 5′-GATA-3′,5′-GATG-3′, and 5′-GATT-3′ sequences on the top strand, results confirmed that mitoABE^{MutH*} indeed worked as an effective editing tool that generated bottom strand edits at all three sites (FIG. 3C-3E). Importantly, none of these sites could be edited by mitoABE^{MutH} because of the absence of the MutH motif (FIGS. 3F-3H).

The MutH motif, 5′-GATC-3′, is a palindromic sequence, thus TALE-MutH* (5′-GATD-3′) can only nick the top strand by the 5′ guanine in certain designs, and only adenine edits on the bottom strand are retained (FIG. 3C-3E). On the other hand, TALE-MutH cannot nick 5′-GATD-3′, resulting in no edits (FIGS. 3F-3H). In addition, MutH* yielded the best editing efficiency when it was located 3 bp away from 5′-GATD-3′ at the right end, which was likely due to the high nicking efficiency (FIGS. 3C-3E). These results confirmed the presumed working principle of the mitoABE^{MutH}, and TALE-MutH* indeed expands its editing scope. There are 23 recognition sites (5′-GATC-3′) of MutH in human mtDNA, but MutH* (5′-GATN-3′) has 485 sites. Consequently, TALE-TadA8e(V106W) could function within a range of 20 bp upstream and downstream of the nick position. Based on this, the proportion of designable mitoABEs in the human mitochondrial genome was estimated. TALE-MutH has a designable targeting range of only about 6% of the mitochondrial genome, while the TALE-MutH* has a range of about 71%, with an average of two 5′-GATN-3′ sites per 40 bp in the mitochondrial genome (FIGS. 3I-J).

### Example 4: Additional Nickases and Use in a mitoABE System

This example demonstrates additional nickases that acan confer functionality to a mitoABE system. In addition, having access to numerous nickases broadens the targetability of the mitoABE system and allows editing of a far greater number of loci within the mitochondrial genome.

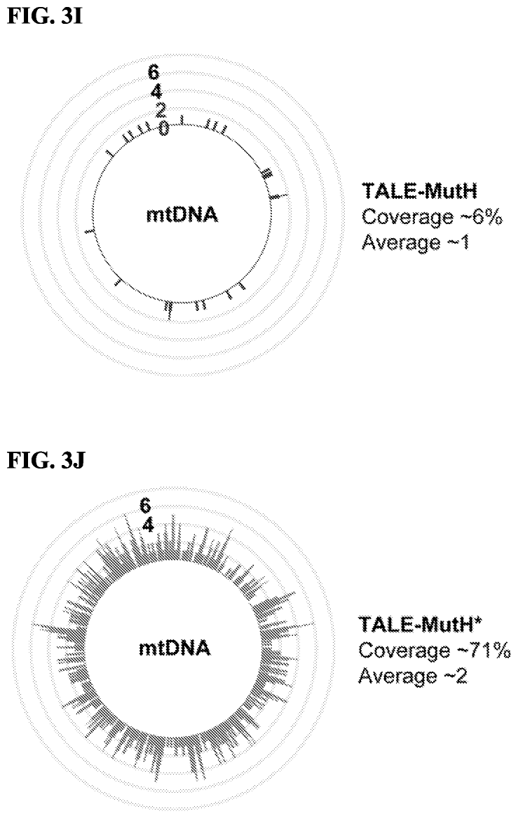

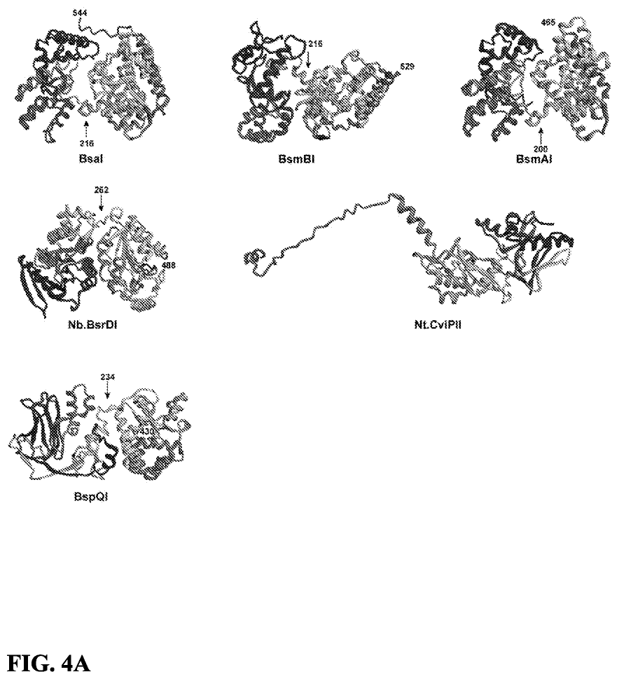

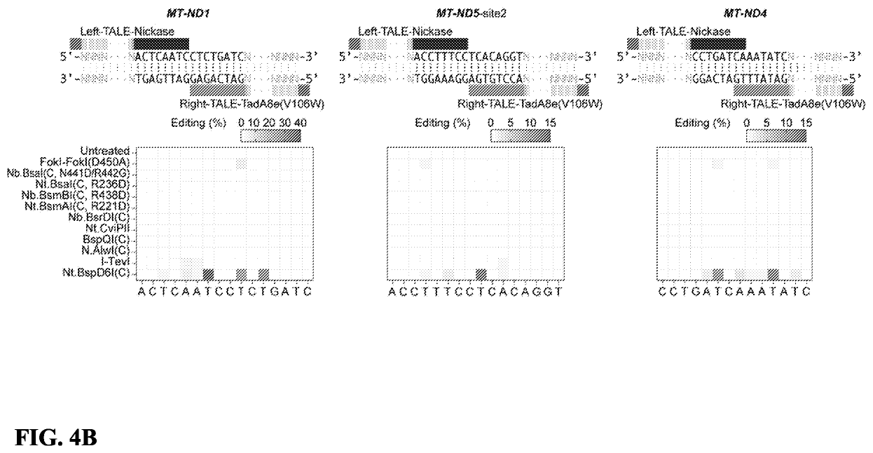

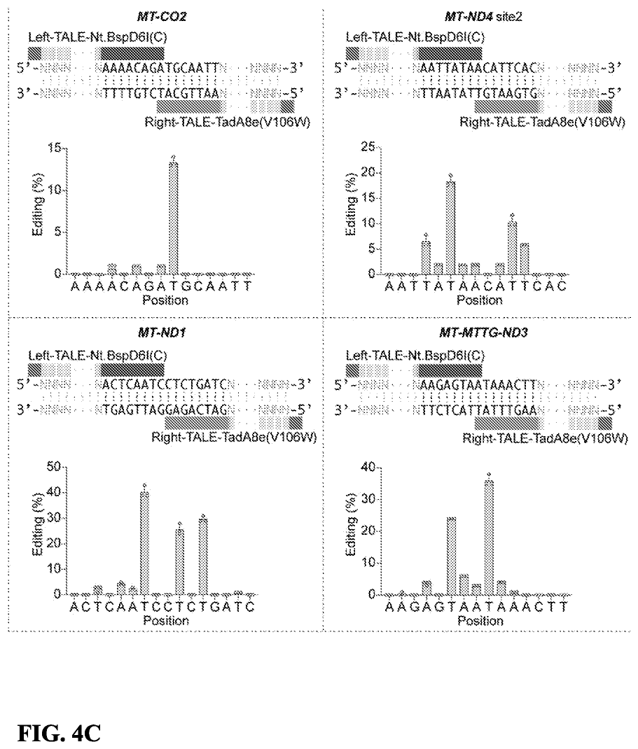

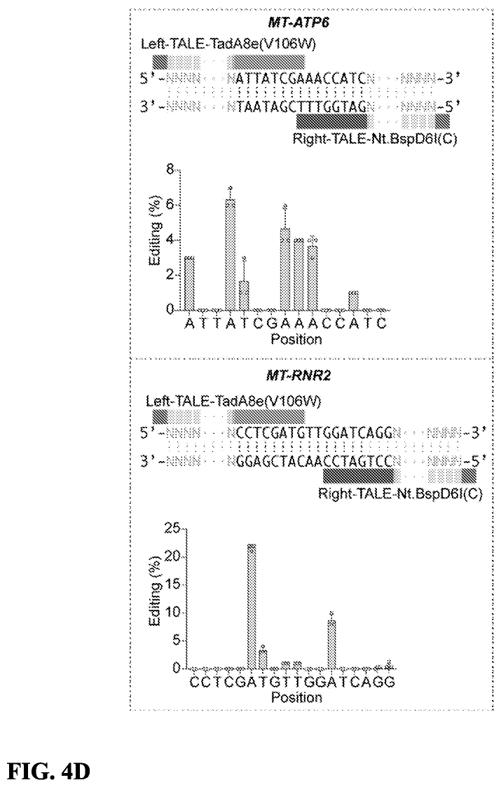

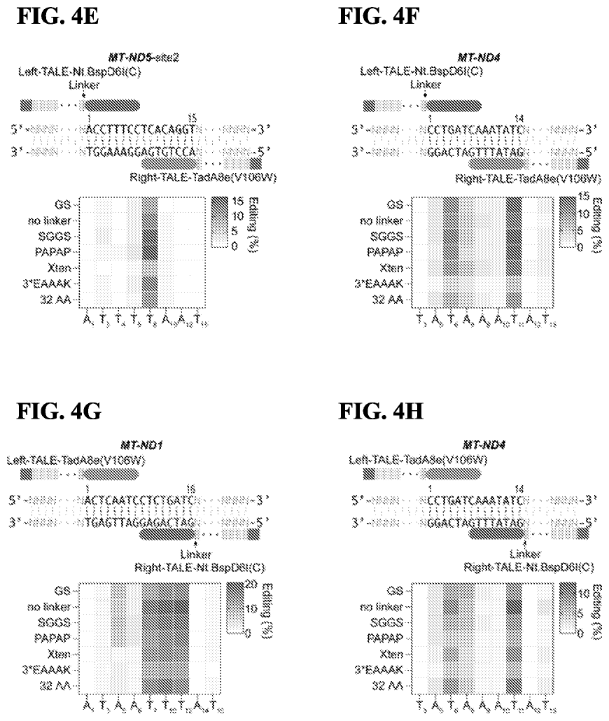

To further broaden the editable scope of mitoABE, multiple enzymes with potential nickase activity were evaluated. Since some nucleases have separate active centers for cutting double-stranded DNA, mutation(s) inactivating one active center might convert the nuclease to a nickase. In particular, the cleavage and recognition domains of type IIS restriction endonuclease are separable, which makes this class of endonuclease an ideal candidate for conversion to a nickase through half-deactivation of their cleavage domains. For enzymes without crystal structures, the cleavage domains were predicted for engineering purposes (FIG. 4A). The MutH component of mitoABE^{MutH} was replaced with the naturally existing nickase Nt.BspD6I(C) and engineered nickases, such as FokI-FokI (D450A), Nb.BsaI (C, N441D/R442G), Nt.BsaI (C, R236D), Nb.BsmBI (C, R438D), Nt.BsmAI (C, R221D), Nb.BsrDI (C), Nt.CviPII (5′-1CCD-3′), BspQI (C), N.AlwI (C), and I-TEV-I (5′-CNNN1G-3′) to verify whether any of these enzymes could nick DNA when fused with an appropriate TALE array. The recognition domains of all enzymes mentioned above were eliminated except for Nt.CviPII (5′-1CCD-3′) and I-TEV-I (5′-CNNN1G-3′), and nickases that don't possess recognition motifs and instead solely rely on the TALE array for recognition were identified.

By fusing the above nickases with Left TALE, their potential editing activities when teamed up with Right-TALE-TadA8e(V106W) were tested (FIG. 4B). The following three editing sites, MT-ND1, MT-ND5 site 2, and MT-ND4, were selected for testing. Among all TALE array-fused candidate nickases, TALE-Nt.BspD6I(C) enabled base editing activity at all three targeted sites when combined with TALE-TadA8e(V106W) (FIG. 4B). Nt.BspD6I is a nickase that can form a heterodimer with BspD6I (the small subunit, 20 kDa) and function as a restriction endonuclease called R.BspD6. The Nt.BspD6I(C) domain, which was fused with a TALE array, is only the C-terminal cleavage domain (382-604 aa). In comparison with TALE-MutH, TALE-Nt.BspD6I(C) showed a certain level of nonstrand-specific editing at the ND4 site (FIG. 4B-4D), possibly due to its imprecise nick on dsDNA. This editing tool will be further referred to as mitoABE^{Nt.BspD6I(C)}.

To further characterize the editing pattern of mitoABE^{Nt.BspD6I(C)}, mitoABE^{Nt.BspD6I(C)} was targeted to a more diverse set of mitochondrial DNA sequences. Among these sites, mitoABE^{Nt.BspD6I(C)} reached up to ~40% editing efficiency with strand specificity (FIG. 4C). In addition, when the TALEs of TALE-Nt.BspD6I(C) and TALE-TadA8e(V106W) were switched, the edited strand was switched reciprocally (FIG. 4D). The linker sequences between TALE and Nt. BspD6I(C) did not affect the editing features of mitoABE^{Nt.BspD6I(C)} (FIGS. 4E-H). From all tested sites, results suggest that TALE-Nt.BspD6I(C) produced the nick on the same DNA strand recognized by itself, resulting in the editing of adenine(s) in the strand recognized by TALE-TadA8e(V106W).

### Example 5: MitoCBE System Using Mitochondrial C-to-T Editing Deaminases

This example demonstrates that by fusing a cytidine deaminase, APOBEC1, to a TALE array and inducing single strand DNA morphology, it is possible to achieve programmed C-to-T mitochondrial DNA editing. This system utilizes the same strand specific mechanism as mitoABE and is termed mitoCBE.

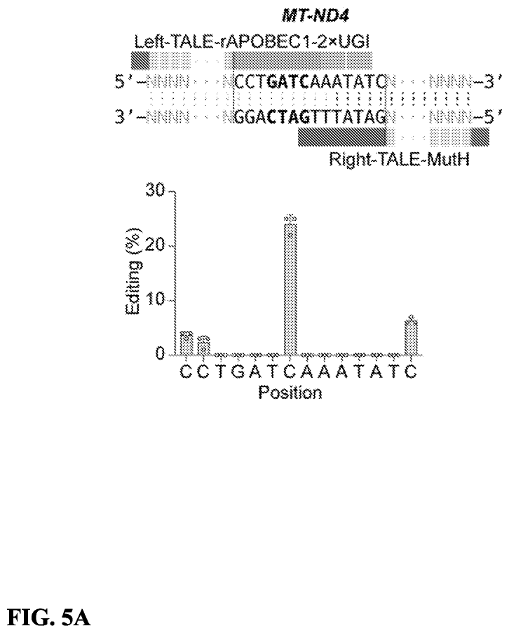

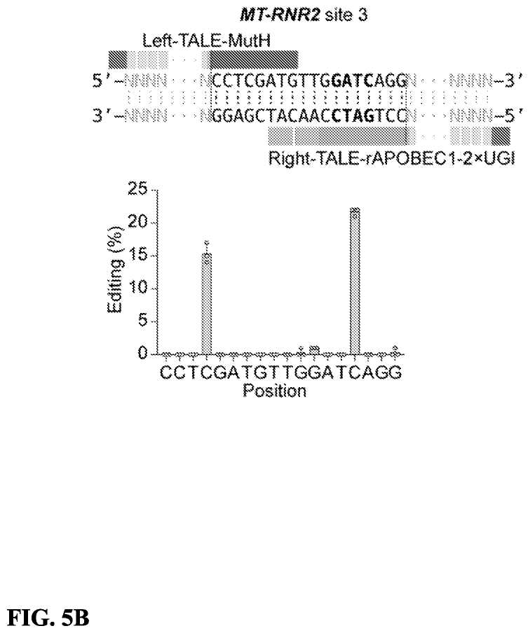

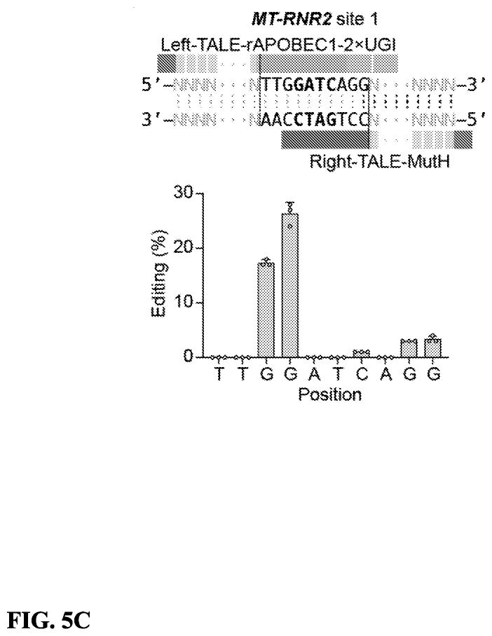

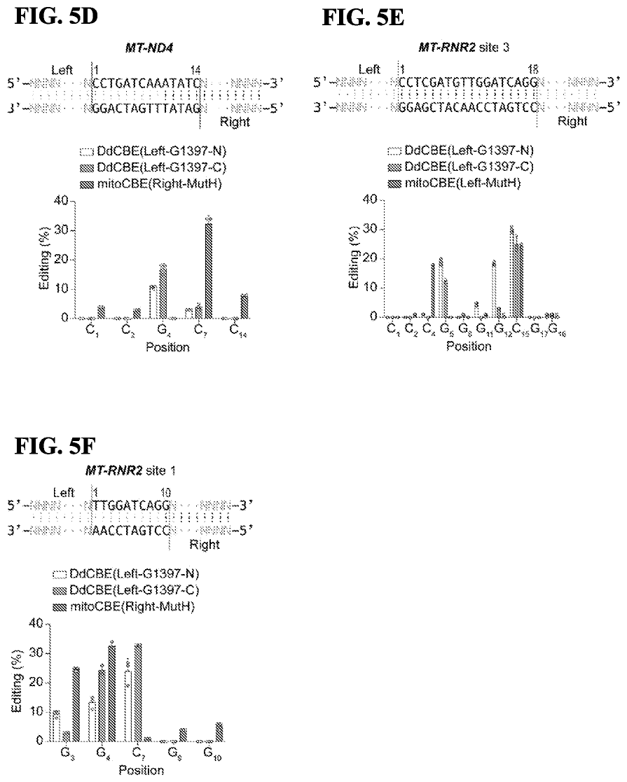

The results of mitoABE suggested that a similar strategy could be readily applied to other types of deaminases, such as APOBEC1, which is responsible for C-to-T editing on ssDNA. By replacing TadA8e(V106W) with rAPOBEC1 fused to a uracil glycosylase inhibitor (UGI), mitochondrial C-to-T editing occurred with a maximum editing efficiency of ~30% using the combination of TALE-rAPOBEC1-2×UGI and TALE-MutH (FIG. 5A-5C). Similar to mitoABE^{MutH}, editing by mitoCBE^{MutH} is also strand specific. For MT-ND4 and MT-RNR2 site 3, the top strands were edited (FIGS. 5A, 5B), and for MT-RNR2 site 1, the bottom strand was edited (FIG. 5C). In comparison, prior mitochondrial C-to-T base editors based on DddA are not strand-specific. In contrast, editing by DbCBEs was not biased towards a specific strand at these three sites (FIG. 5D-F).

### Example 6: Monomeric mitoABE System Enabling A-to-G Conversion

This example demonstrates that encoding monomeric mitoABE and mitoCBE systems with a single TALE array can result in efficient A-to-G and C-to-T editing, respectively.

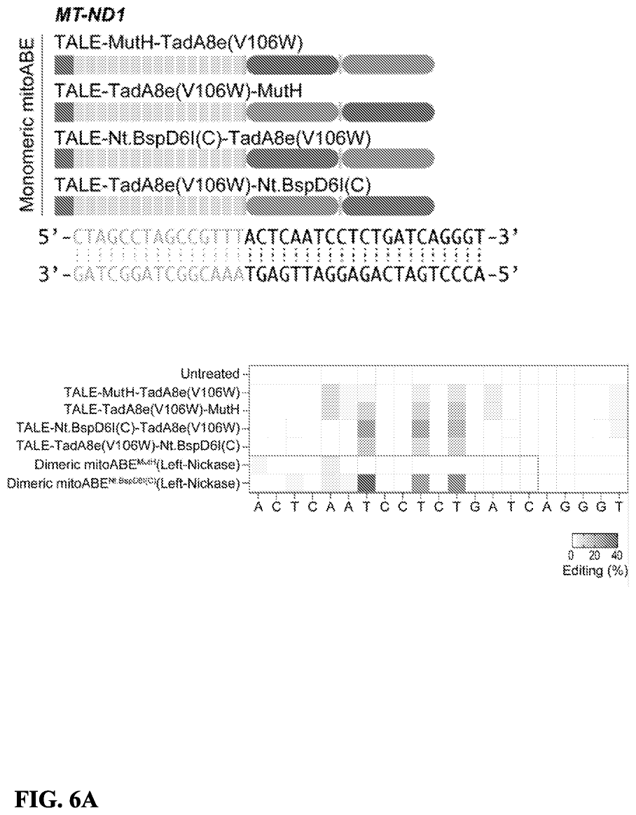

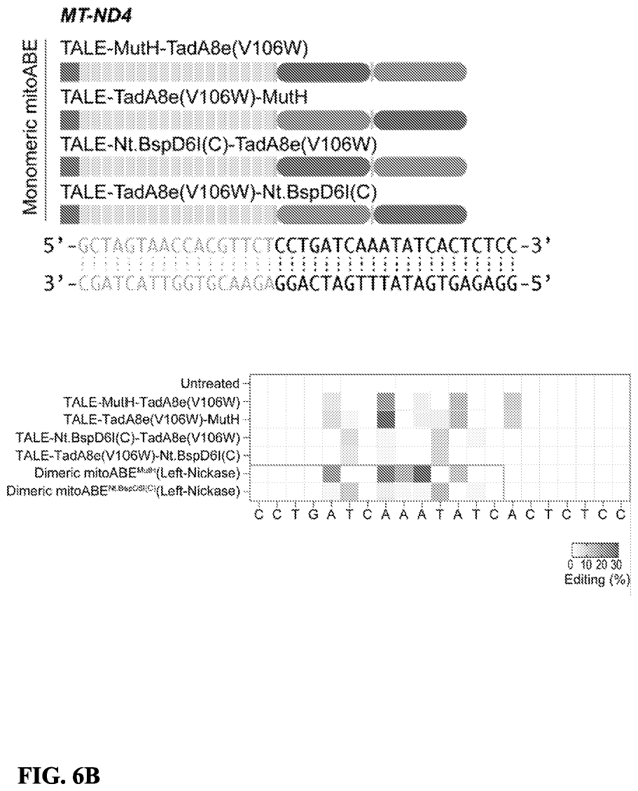

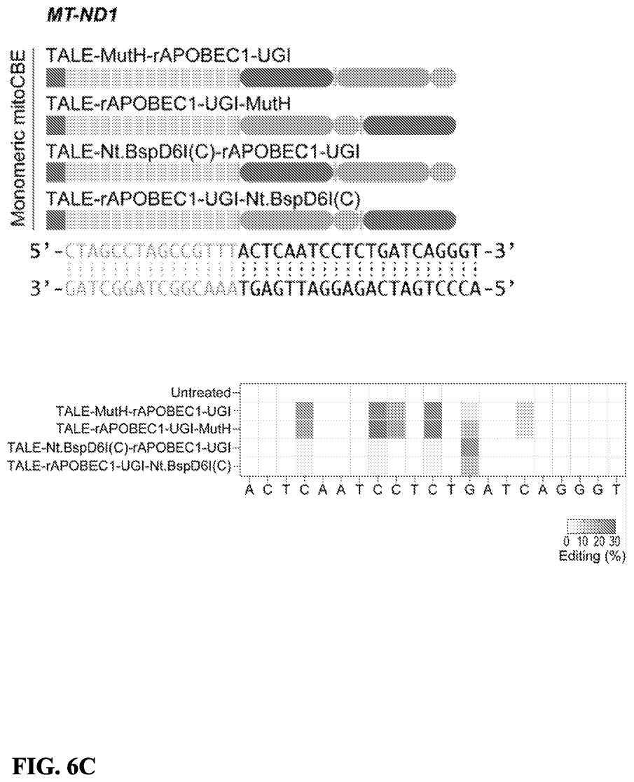

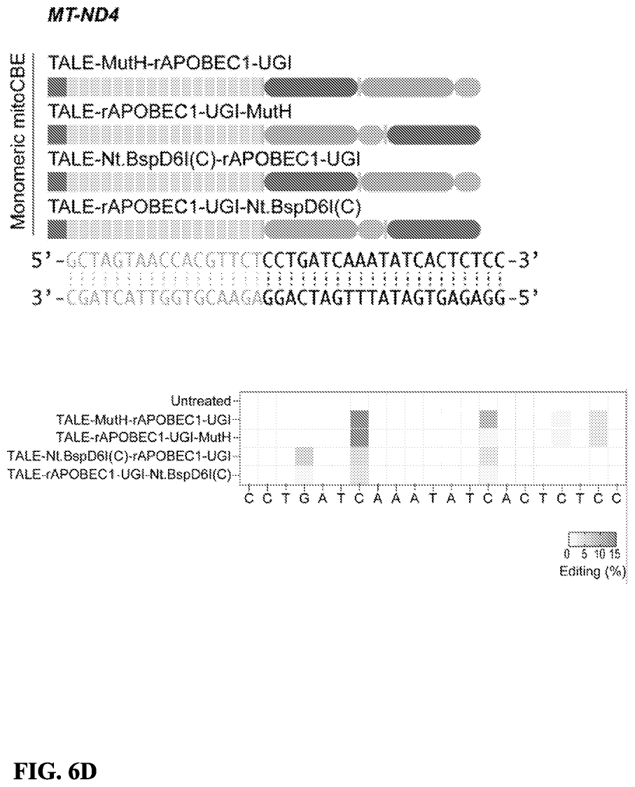

Although it is beneficial to have nickase and deaminase domains in two separate TALE arrays, it is tempting to test if they could still work when fused with the same TALE array. Four versions of such mitoABEs were designed, TALE-MutH-TadA8e(V106W), TALE-TadA8e(V106W)-MutH, and TALE-Nt.BspD6I(C)-TadA8e(V106W) and TALE-TadA8e(V106W)-Nt.BspD6I(C). Monomeric mitoABE, mitoABE^{MutH} and mitoABE^{Nt.BspD6I(C)} all enabled efficient A-to-G editing (FIGS. 6A, 6B). The monomeric versions of mitoABE^{MutH} achieved higher editing efficiency at the MT-ND1 target site compared to dimeric mitoABE^{MutH}, while the dimeric type of mitoABE^{Nt.BspD6I(C)} yielded higher editing efficiency. Moreover, monomeric mitoABEs have a wider editing window compared to dimeric mitoABEs, with a consistent strand-preference observed for both types within the editing windows (FIGS. 6A, 6B). The smaller size of monomeric mitoBEs makes them easier to deliver, especially when using AAV as a vector. In addition, monomeric mitoCBEs (mitoCBE^{MutH} and mitoCBE^{Nt.BspD6I(C)} were successfully constructed, and achieved efficient C-to-T editing at targeted sites (FIG. 6C, 6D).

### Example 7: Editing Specificity of mitoBE Systems

This example demonstrates the editing specificity and safety of mitoBE systems. Using mitochondrial DNA sequencing methods, this example demonstrates that the mitoABE system does not generate off-target A-to-G mitochondrial edits or mitochondrial copy number alterations.

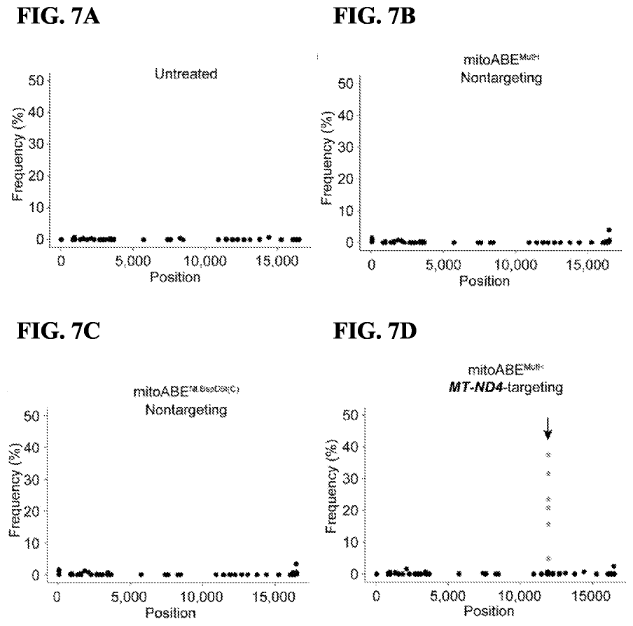

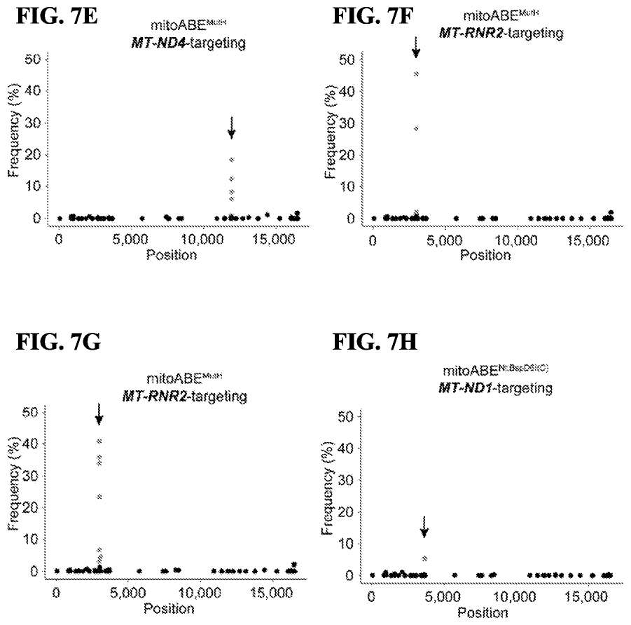

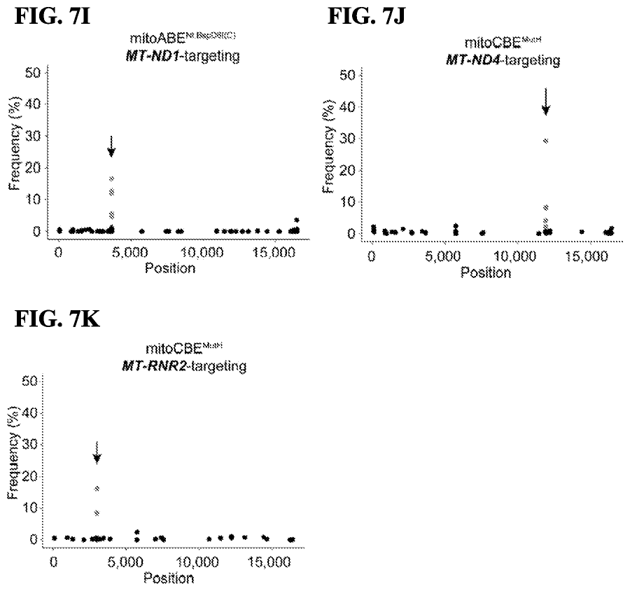

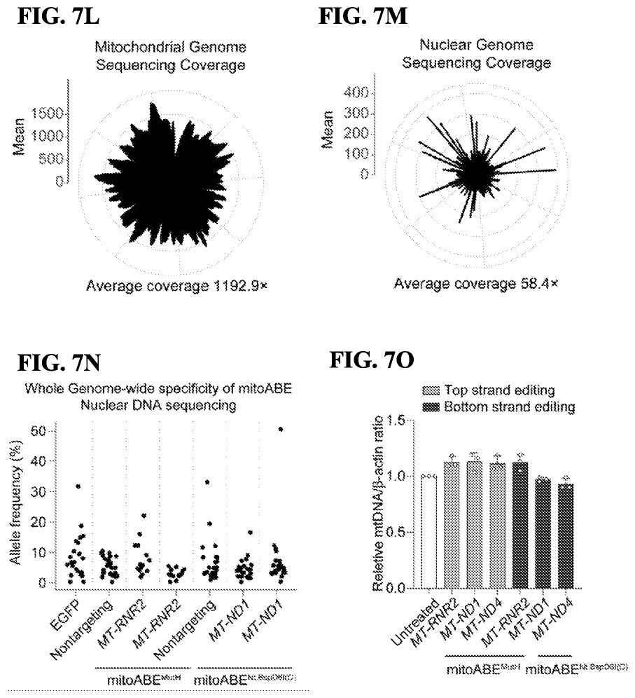

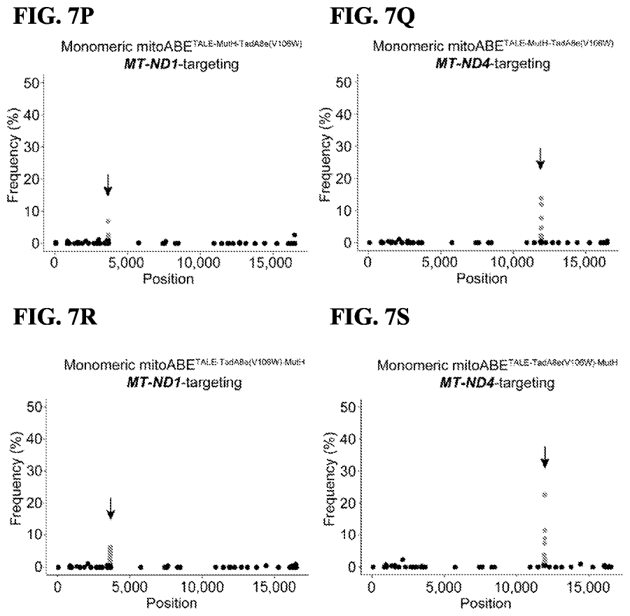

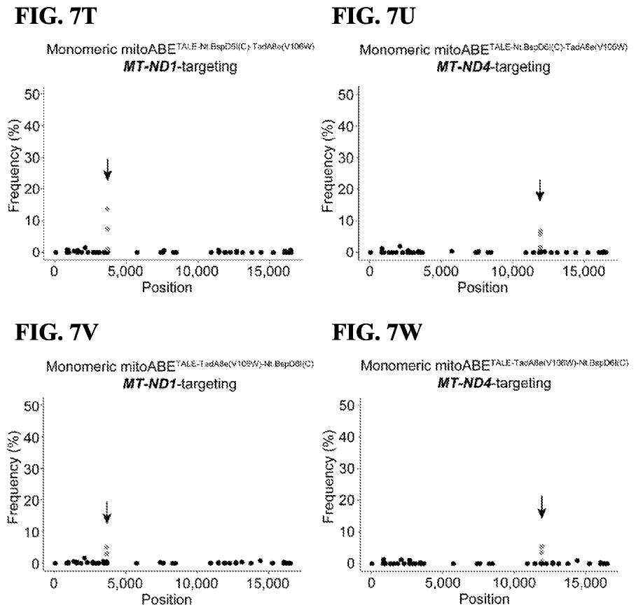

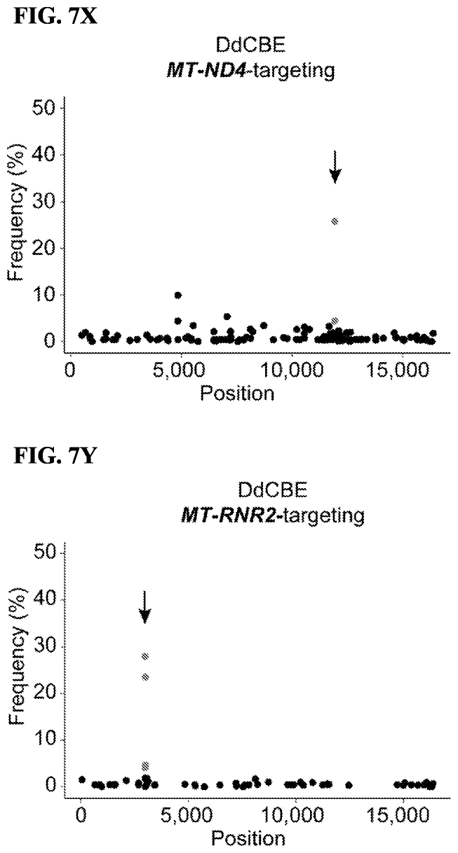

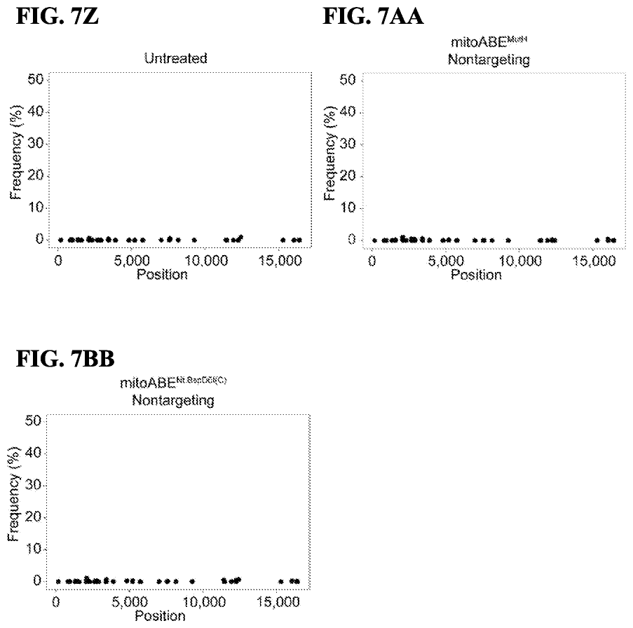

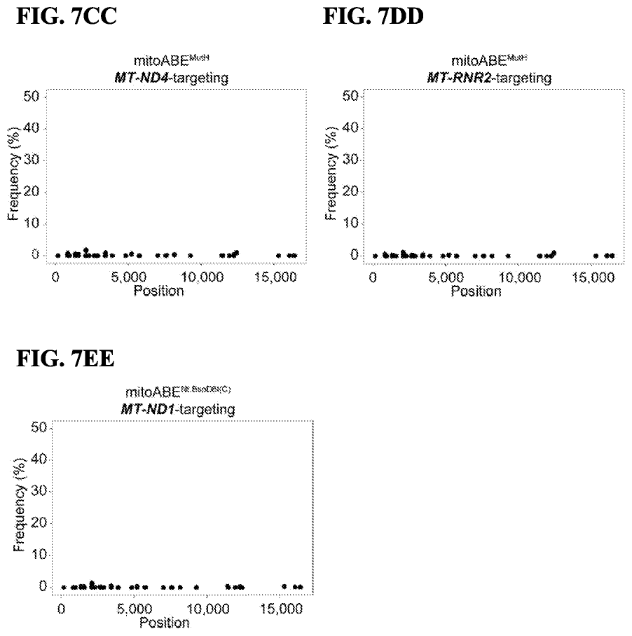

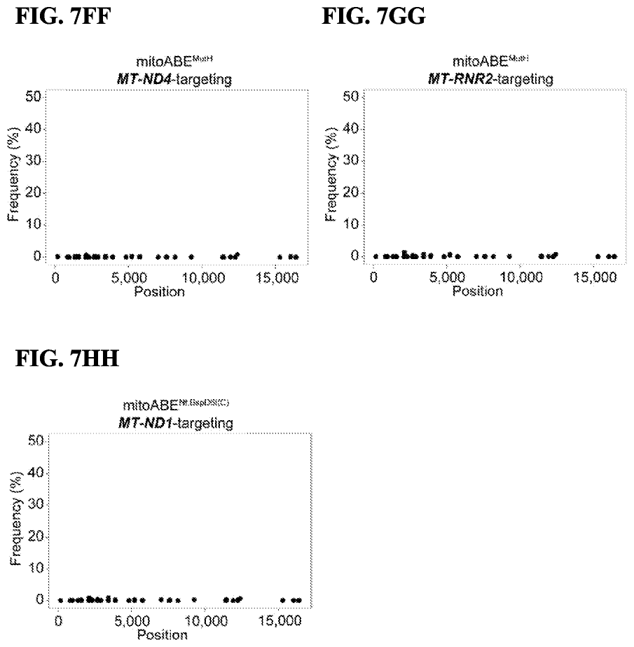

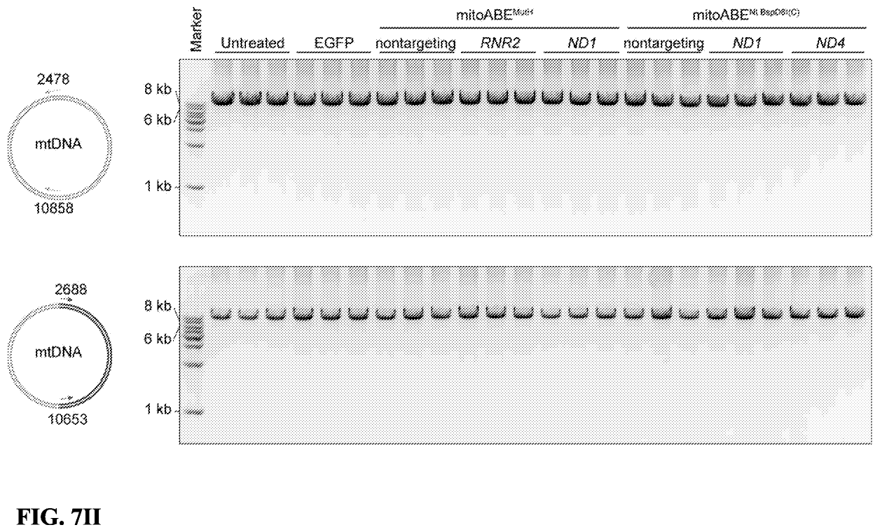

Mitochondrial DNA sequencing analysis was performed to evaluate the editing specificity of mitoBE. HEK293T cells transfected with either mitoABE^{MuH} or mitoABE^{Nt.BspD6I(C)}-expressing plasmids were subjected to mitochondrial DNA sequencing analysis, in which the untreated group (FIG. 7A) and non-targeting groups, including mitoABE^{MutH} and mitoABE^{Nt.BspD6I(C)} without an associated TALE array (FIGS. 7B, 7C), were used as a control. The mean sequencing coverage across the mitochondrial genome was approximately 1193× (FIG. 7L). Mitochondrial DNA sequencing analysis detected only on-target editing compared to the controls (untreaded and non-targeting), and did not detect nonspecific editing in any experimental group (FIGS. 7A-7I). Of note, the fact that there was no difference between the nontargeting (FIGS. 7B, 7C) and untreated groups (FIG. 7A) suggests that the free form of either TALE-deaminase or TALE-nickase does not cause unwanted off-target effects. We also assessed the editing specificity of monomeric mitoABEs (monomeric mitoABE^{MutH} and mitoABE^{Nt.BspD6I(C)} and found their specificity to be comparable to that of dimeric mitoABEs (FIGS. 7P-7W). This suggests that both monomeric and dimeric mitoABEs display high specificity when editing the mitochondrial gemone. Additionally, we compared the off-target editing of mitoCBEs to that of DdCBEs with the same TALE array and found that mitoCBEs induced lower off-target editing in the mitochondrial genome, particularly at the MT-ND4-targeted site (FIGS. 7J, 7K, 7X, 7Y). These results demonstrated that mitoBEs represent reliable mitochondrial editing tools with minimal off-target editing on mitochondrial DNA.

Mitochondrial gene editing tools such as DdCBEs are known to cause off-target effects in the nuclear genome. To investigate whether mitoBEs also have off-target effects in the nucleus, whole-genome sequencing (with an average coverage of ~58.4×) was performed and overall off-target editing in the targeting group (including mitoABE^{MutH} and mitoABE^{Nt.BspD6I(C)}) was compared to that of the EGFP and non-targeting control groups. No significant difference between the targeted groups and the control groups was found (FIGS. 6M, 6N). Furthermore, the presence of TALE-dependent off-target effects was analyzed using the whole genome sequencing data and no off-target editing was found within ±200 bp of TALE-array binding sequences (including 0 or 1 mismatch) in the nuclear genome. These findings suggest that mitoBEs exhibit low off-target effects in the nuclear genome.

To further evaluate the effect of mitoABEs on mitochondria, the copy number and integrity of mtDNA was measured. Mitochondrial DNA sequencing data was used to search for the presence of indels within mitochondrial DNA. No difference was found between the targeted group (FIGS. 7CC-7HH) and the controls (FIGS. 7Z-7BB). Real-time quantitative PCR and long-range PCR analysis demonstrated that the copy number and integrity of mitochondrial DNA in the targeted group remained the same as those in the controls (FIGS. 7O, 7II). Collectively, mitoABEs showed high specificity in human cells.

### Example 8: Circular RNA-Encoded mitoABE Systems Enable Strand-Specific Editing in Multiple Cell Lines

This example demonstrates a circular RNA delivery system to efficiently induce mitochondrial DNA base editing in various human cell lines.

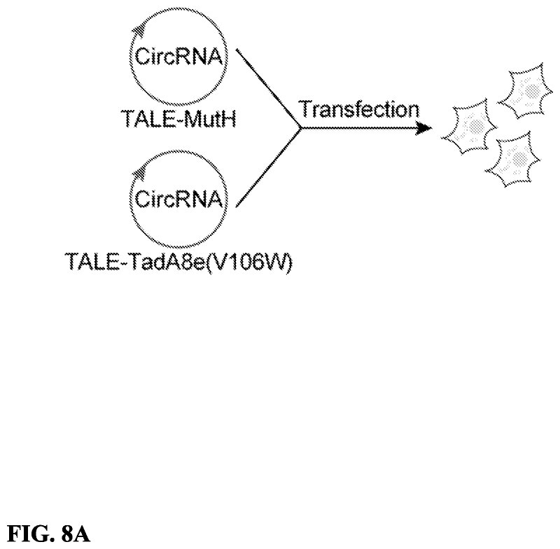

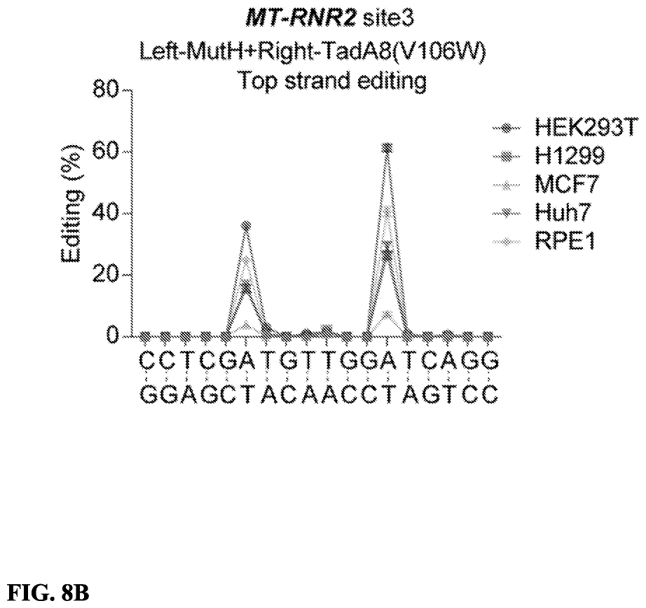

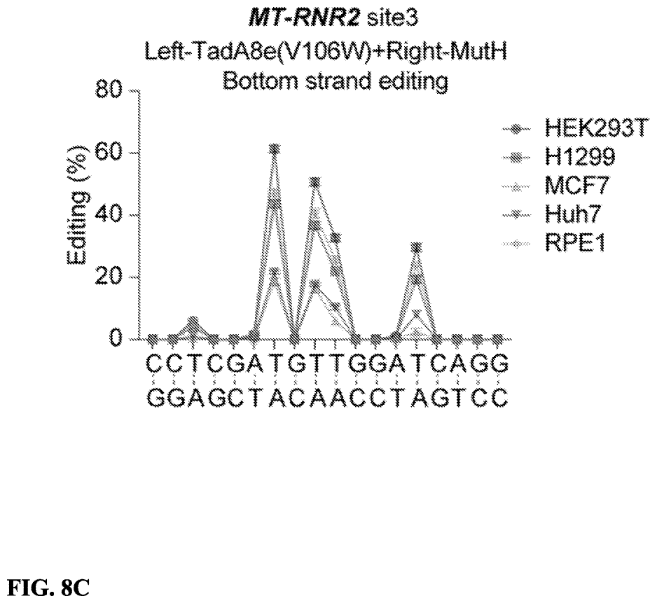

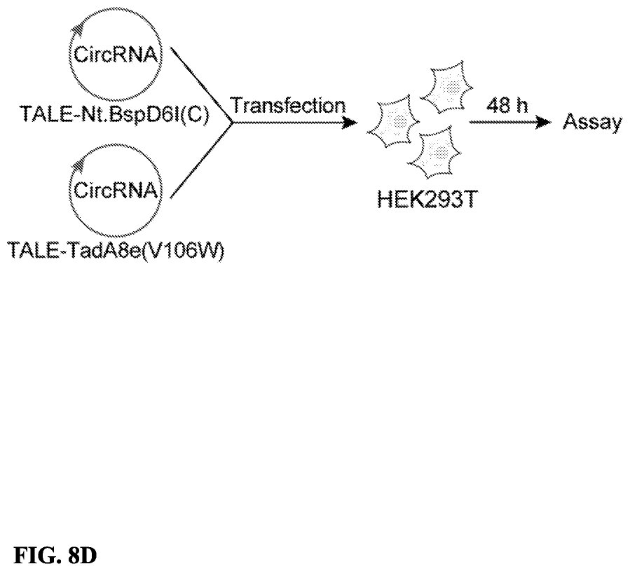

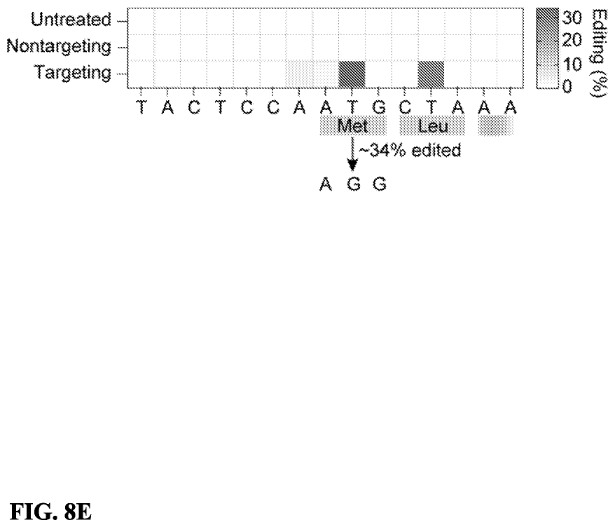

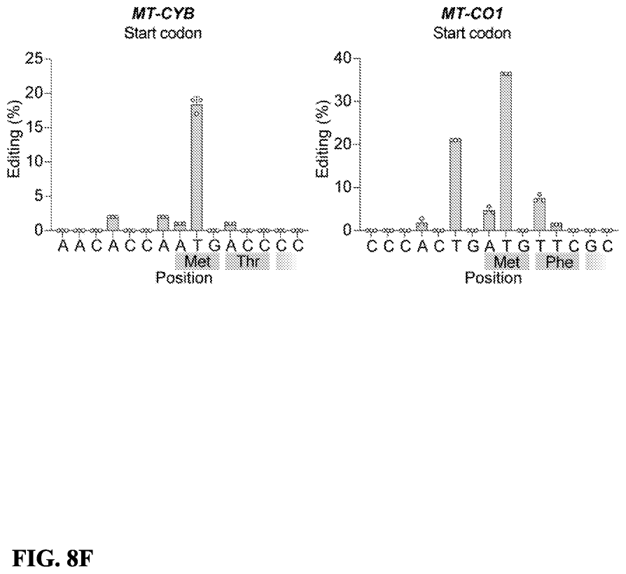

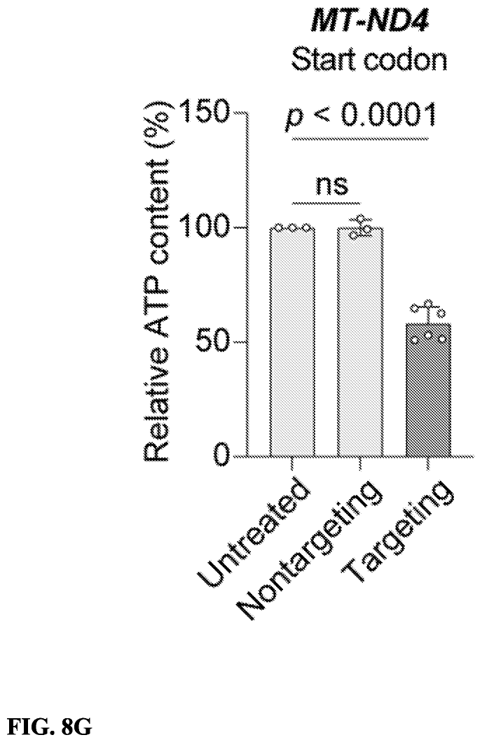

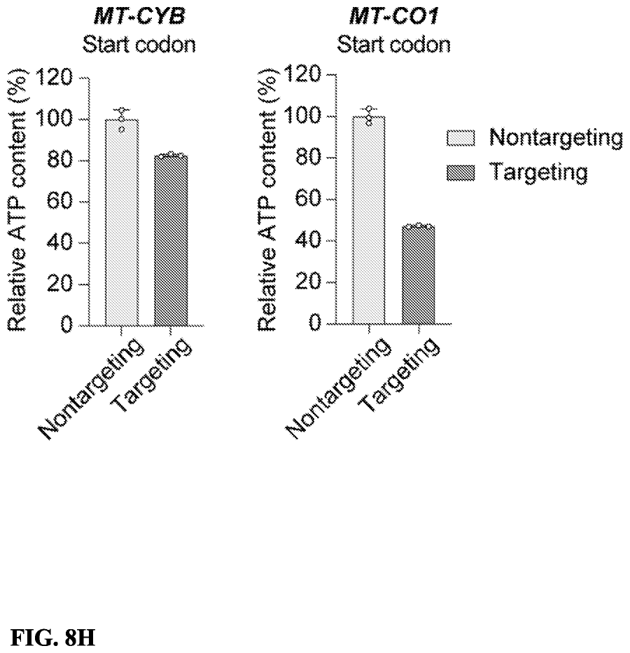

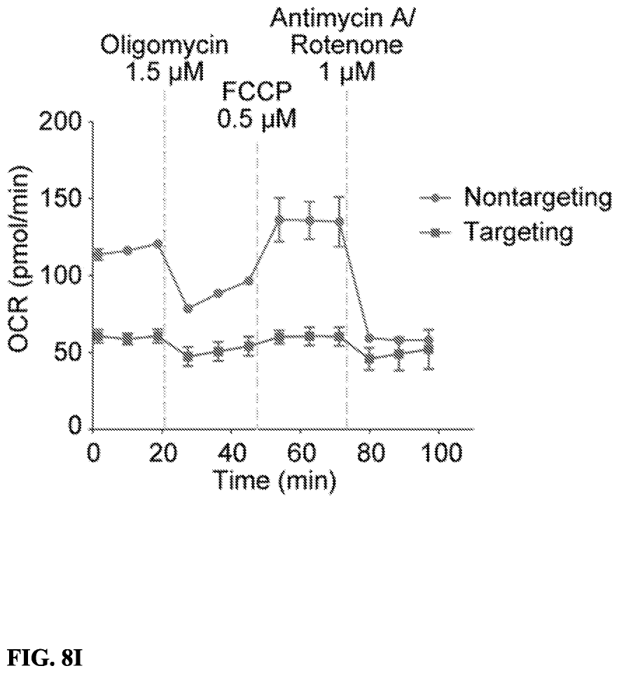

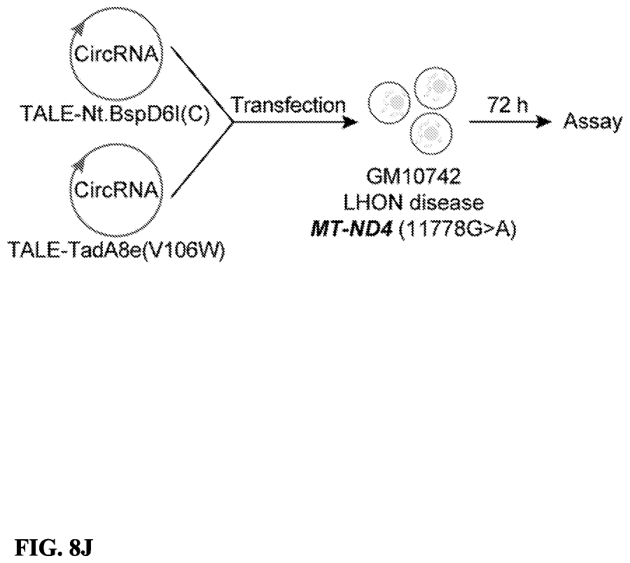

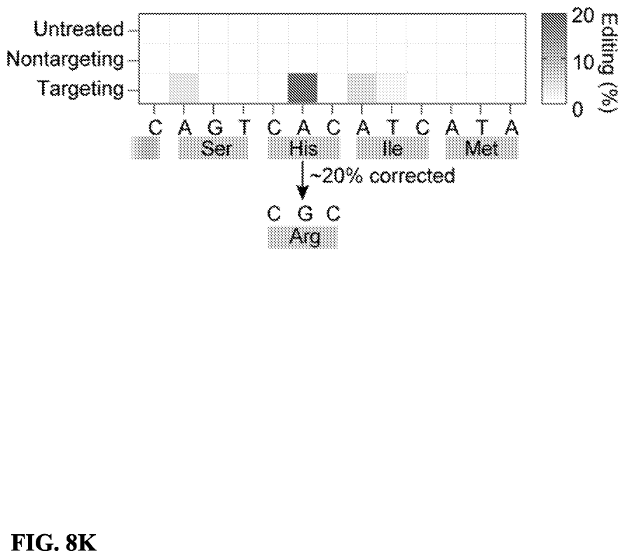

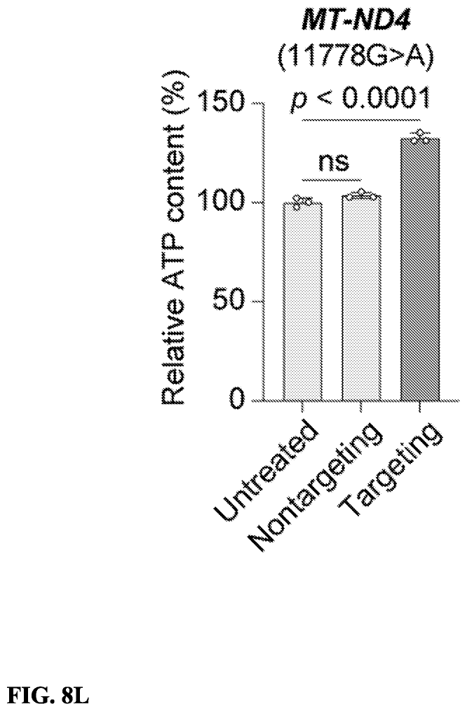

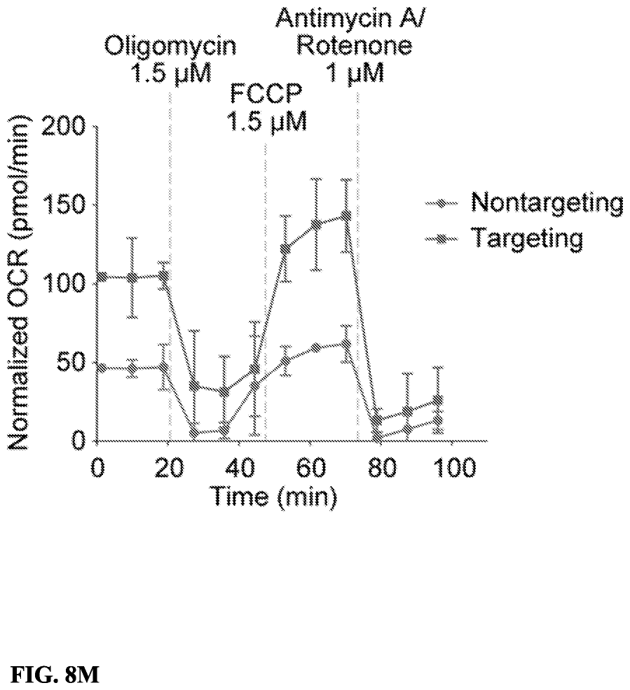

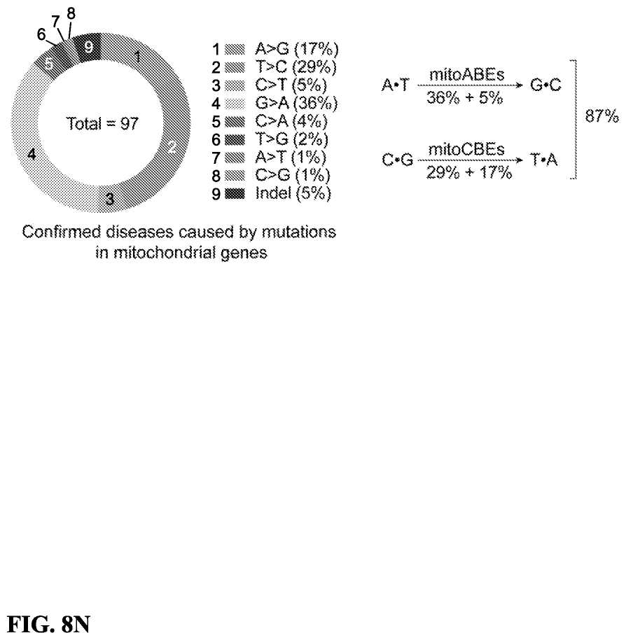

Treatment of disease by direct delivery of RNA shows good potential. Since mitoABE, unlike the CRISPR system, does not require RNA components to function, mitochondrial editing was tested using circular RNA to encode mitoABE. Circular RNA-encoded mitoABE conferred strand-specific editing in various human cell types, including H1299, MCF7, Huh7, and RPE1, indicating that mitoABEs are versatile tools compatible with various delivery routes to achieve efficient and precise mitochondrial DNA base editing (FIGS. 8A-C).

### Example 9: Editing Start Codons of Mitochondrial Genes Perturbs the Function of the Respiratory Chain

This example demonstrates that a mitoABE system can effectively edit mitochondrial DNA to create cell-based models for mitochondrial dysfunction.

Mitochondrial diseases are a group of genetic disorders caused by mutations in either nuclear or mitochondrial DNA, which are characterized by defects in oxidative phosphorylation. Approximately 90% of mitochondrial genetic disorders caused by mitochondrial DNA mutations are due to single base mutations of the mitochondrial coding genes. The leading cause of these genetic disorders is a decrease in ATP production due to the defective assembly of the mitochondrial respiratory complex. Using circRNA-encoded mitoABE to target the start codons of three genes in HEK293T cells, these new editing tools were successfully able to generate phenotypes mimicking real mitochondrial diseases (FIG. 7D). Three target loci were chosen MT-ND4, MT-CYB, and MT-CO1, which encode proteins that are components of mitochondrial complex I, mitochondrial complex III, and mitochondrial complex IV, respectively. Effective editing by mitoABEs altered all ATG start codons at these three loci by changing T (actually edited A on the noncoding strand) to C, with editing efficiencies of 34%, 18% and 36%, respectively (FIGS. 7E, 7F). By measuring the level of intracellular ATP content, editing at all three loci resulted in a decrease in intracellular ATP content (FIGS. 7G, 7H). In addition, the cells with the edited start codon of MT-ND4 exhibited a low rate of respiration oxygen consumption (FIG. 7I). Collectively, these results demonstrated that mitoABEs could effectively edit DNA to create mitochondrial disease models with oxidative respiratory defects.

### Example 10: Correcting Mitochondrial Pathogenic DNA Mutations Via a mitoABE System

This example demonstrates that the mitoABE system is capable of correcting inherited mitochondrial mutations which contribute to mitochondrial disease. Importantly, when correcting mutations within mitochondrial oxidative respiratory chain proteins, physiological efficacy can be demonstrated by measuring ATP content and respiratory oxygen consumption rates in targeted cells.

Leber hereditary optic neuropathy (LHON) is the most common inherited mitochondrial disease that affects young adults, ultimately leading to acute or subacute blindness. LHON is usually caused by one of three pathogenic mitochondrial DNA (mtDNA) point mutations. These mutations are located at nucleotide positions 11778 G to A, 3460 G to A, and 14484 T to C in the MT-ND4, MT-ND1, and MT-ND6 subunit genes of the mitochondrial oxidative respiratory chain complex I, respectively. The 11778 G to A mutation located at MT-ND4 changes the highly conserved arginine to histidine (R340H), which accounts for 50% of LHON cases among Caucasians and over 90% of the cases in Asians. Using circRNA-encoded mitoABEs to target LHON patient-derived GM10742 cells³⁷, a repair efficiency of 20% was detected on pathogenic mutation (G11778A) (FIGS. 7J, K). Importantly, such correction through mitoABE resulted in a significant increase in ATP content and respiratory oxygen consumption rate in GM10742 cells (FIG. 7L, M). This result demonstrates the strong therapeutic potential of mitoABE in treating LHON diseases and potentially many other mitochondrial genetic disorders caused by SNPs. Currently, 97 mtDNA mutations have been linked to human diseases, with the majority being point mutations (MITOMAP, Table 1). Of these, 46% are attributed to A·T to G·C mutations, while 41% are caused by C·G to T·A mutations. Theoretically, mitoBEs have the potential to model or correct these disease-associated mutations (FIG. 8N).

TABLE 1
mtDNA Mutations Linked to Human Disease

| Index | Locus | Associated Diseases | Allele | Position |
|---|---|---|---|---|
| 1 | MT-TF | MELAS/MM & EXIT | m.583G > A | 583 |
| 2 | MT-TF | Maternally inherited epilepsy/mito | m.616T > C | 616 |
| | | tubulointerstitial kidney disease (MITKD)/ | | |
| | | Gitelman-like syndrome | | |
| 3 | MT- | DEAF | m.1494C > T | 1494 |
| | RNR1 | | | |
| 4 | MT- | DEAF; autism spectrum intellectual disability; | m.1555A > G | 1555 |
| | RNR1 | possibly antiatherosclerotic | | |
| 5 | MT-TV | AMDF | m.1606G > A | 1606 |
| 6 | MT-TV | MNGIE-like disease/MELAS | m.1630A > G | 1630 |
| 7 | MT-TV | Leigh Syndrome/HCM/MELAS | m.1644G > A | 1644 |
| 8 | MT-TL1 | MELAS/Leigh Syndrome/DMDF/MIDD/ | m.3243A > G | 3243 |
| | | SNHL/CPEO/MM/FSGS/ASD/ | | |
| | | Cardiac + multi-organ dysfunction | | |
| 9 | MT-TL1 | MM/MELAS/SNHL/CPEO | m.3243A > T | 3243 |
| 10 | MT-TL1 | MELAS; possible atherosclerosis risk | m.3256C > T | 3256 |
| 11 | MT-TL1 | MELAS/Myopathy | m.3258T > C | 3258 |
| 12 | MT-TL1 | MMC/MELAS | m.3260A > G | 3260 |
| 13 | MT-TL1 | MELAS/DM | m.3271T > C | 3271 |
| 14 | MT-TL1 | PEM/retinal dystrophy in MELAS | m.3273delT | 3273 |
| 15 | MT-TL1 | Myopathy | m.3280A > G | 3280 |
| 16 | MT-TL1 | MELAS/Myopathy/Deafness + Cognitive | m.3291T > C | 3291 |
| | | Impairment | | |
| 17 | MT-TL1 | MM | m.3302A > G | 3302 |
| 18 | MT-TL1 | MMC | m.3303C > T | 3303 |
| 19 | MT-ND1 | LHON MELAS overlap | m.3376G > A | 3376 |
| 20 | MT-ND1 | LHON | m.3460G > A | 3460 |
| 21 | MT-ND1 | LHON | m.3635G > A | 3635 |
| 22 | MT-ND1 | MELAS/Leigh Syndrome/LDYT/BSN | m.3697G > A | 3697 |
| 23 | MT-ND1 | LHON | m.3700G > A | 3700 |
| 24 | MT-ND1 | LHON | m.3733G > A | 3733 |
| 25 | MT-ND1 | Progressive Encephalomyopathy/Leigh | m.3890G > A | 3890 |
| | | Syndrome/Optic Atrophy | | |
| 26 | MT-ND1 | EXIT + myalgia/severe LA + cardiac/3-MGA | m.3902_3908- | 3902 |
| | | aciduria/nephropathy + deafness + diabetes | ACCTTGCinv | |
| 27 | MT-ND1 | LHON/Leigh-like phenotype | m.4171C > A | 4171 |
| 28 | MT-TI | CPEO/MS | m.4298G > A | 4298 |
| 29 | MT-TI | MICM | m.4300A > G | 4300 |
| 30 | MT-TI | CPEO | m.4308G > A | 4308 |
| 31 | MT-TQ | Encephalopathy/MELAS | m.4332G > A | 4332 |
| 32 | MT-TM | Myopathy/MELAS/Leigh Syndrome | m.4450G > A | 4450 |
| 33 | MT-TW | Mitochondrial myopathy | m.5521G > A | 5521 |
| 34 | MT-TW | Leigh Syndrome | m.5537_5538insT | 5537 |
| 35 | MT-TA | Myopathy | m.5650G > A | 5650 |
| 36 | MT-TN | CPEO + ptosis + proximal myopathy | m.5690A > G | 5690 |
| 37 | MT-TN | CPEO/MM | m.5703G > A | 5703 |
| 38 | MT-TN | Multiorgan failure/myopathy | m.5728T > C | 5728 |
| 39 | MT-CO1 | SNHL | m. 7445A > G | 7445 |
| 40 | MT-TS1 | SNHL | m. 7445A > G | 7445 |
| | precursor | | | |
| 41 | MT-TS1 | PEM/AMDF/Motor neuron disease-like | m.7471_7472insC | 7471 |
| 42 | MT-TS1 | MM/EXIT | m.7497G > A | 7497 |
| 43 | MT-TS1 | SNHL | m.7510T > C | 7510 |
| 44 | MT-TS1 | SNHL/Deafness | m.7511T > C | 7511 |
| 45 | MT-TK | Severe adult-onset multisymptom myopathy/ | m.8306T > C | 8306 |
| | | Myoclonic epilepsy | | |
| 46 | MT-TK | MNGIE/Progressive mito cytopathy | m.8313G > A | 8313 |
| 47 | MT-TK | Myopathy/Exercise Intolerance/Eye | m.8340G > A | 8340 |
| | | disease + SNHL | | |
| 48 | MT-TK | MERRF; Other - LD/Depressive mood | m.8344A > G | 8344 |
| | | disorder/leukoencephalopathy/HiCM | | |
| 49 | MT-TK | MERRF | m.8356T > C | 8356 |
| 50 | MT-TK | MICM + DEAF/MERRF/Autism/Leigh | m.8363G > A | 8363 |
| | | Syndrome/Ataxia | | |
| 51 | MT- | Infantile cardiomyopathy/hyperammonemia | m.8528T > C | 8528 |
| | ATP8/6 | | | |
| 52 | MT-ATP6 | BSN/Leigh syndrome | m.8851T > C | 8851 |
| 53 | MT-ATP6 | Mitochondrial myopathy, lactic acidosis and | m.8969G > A | 8969 |
| | | sideroblastic anemia (MLASA)/IgG | | |
| | | nephropathy | | |
| 54 | MT-ATP6 | NARP/Leigh Disease/MILS/other | m.8993T > C | 8993 |
| 55 | MT-ATP6 | NARP/Leigh Disease/MILS/other | m.8993T > G | 8993 |
| 56 | MT-ATP6 | Ataxia syndromes | m.9035T > C | 9035 |
| 57 | MT-ATP6 | MIDD, renal insufficiency | m.9155A > G | 9155 |
| 58 | MT-ATP6 | FBSN/Leigh Disease/Spinocerebellar Ataxia | m.9176T > C | 9176 |
| 59 | MT-ATP6 | Leigh Disease/Spastic Paraplegia/ | m.9176T > G | 9176 |
| | | Spinocerebellar Ataxia | | |
| 60 | MT-ATP6 | Leigh Disease/Ataxia syndromes/NARP-like | m.9185T > C | 9185 |
| | | disease/Episodic weakness and Charcot-Marie- | | |
| | | Tooth | | |
| 61 | MT-ATP6 | Leigh Disease | m.9191T > C | 9191 |
| 62 | MT-ATP6 | Encephalopathy/Seizures/Lacticacidemia | m.9205_9206- | 9205 |
| | | | delTA | |
| 63 | MT-TG | PEM | m.10010T > C | 10010 |
| 64 | MT-ND3 | Leigh Disease/MELAS | m.10158T > C | 10158 |
| 65 | MT-ND3 | Leigh Disease/Leigh-like Disease/ESOC | m.10191T > C | 10191 |
| 66 | MT-ND3 | Leigh Disease/Dystonia/Stroke/LDYT | m.10197G > A | 10197 |
| 67 | MT- | LHON | m.10663T > C | 10663 |
| | ND4L | | | |
| 68 | MT-ND4 | Leigh Disease | m.11777C > A | 11777 |
| 69 | MT-ND4 | LHON/Progressive Dystonia | m.11778G > A | 11778 |
| 70 | MT-TH | MERRF-MELAS/Encephalopathy | m.12147G > A | 12147 |
| 71 | MT-TH | Maternally inherited non-syndromic deafness | m.12201T > C | 12201 |
| 72 | MT-TS2 | DMDF/RP + SNHL | m.12258C > A | 12258 |
| 73 | MT-TL2 | CPEO | m.12276G > A | 12276 |
| 74 | MT-TL2 | CPEO/EXIT + Ophthalmoplegia | m. 12294G > A | 12294 |
| 75 | MT-TL2 | CPEO/KSS/possible carotid atherosclerosis | m.12315G > A | 12315 |
| | | risk, trend toward myocardial infarction risk | | |
| 76 | MT-TL2 | CPEO | m.12316G > A | 12316 |
| 77 | MT-ND5 | Leigh Disease | m.12706T > C | 12706 |
| 78 | MT-ND5 | Optic neuropathy/retinopathy/LD | m.13042G > A | 13042 |
| 79 | MT-ND5 | LHON | m.13051G > A | 13051 |
| 80 | MT-ND5 | Ataxia + PEO/MELAS, LD, LHON, myoclonus, | m.13094T > C | 13094 |
| | | fatigue | | |
| 81 | MT-ND5 | LHON | m.13379A > G | 13379 |
| 82 | MT-ND5 | Leigh Disease/MELAS/LHON-MELAS | m.13513G > A | 13513 |
| | | Overlap Syndrome/negative association w | | |
| | | Carotid Atherosclerosis | | |
| 83 | MT-ND5 | Leigh Disease/MELAS/Ca2+ downregulation | m.13514A > G | 13514 |
| 84 | MT-ND6 | MELAS/Leigh Disease | m.14453G > A | 14453 |
| 85 | MT-ND6 | LDYT/Leigh Disease/dystonia/carotid | m.14459G > A | 14459 |
| | | atherosclerosis risk | | |
| 86 | MT-ND6 | LHON | m.14482C > A | 14482 |
| 87 | MT-ND6 | LHON | m.14482C > G | 14482 |
| 88 | MT-ND6 | LHON | m.14484T > C | 14484 |
| 89 | MT-ND6 | Dystonia/Leigh Disease/ataxia/ptosis/ | m.14487T > C | 14487 |
| | | epilepsy | | |
| 90 | MT-ND6 | LHON | m.14495A > G | 14495 |
| 91 | MT-ND6 | LHON | m.14568C > T | 14568 |
| 92 | MT-TE | Reversible COX deficiency myopathy | m.14674T > C | 14674 |
| 93 | MT-TE | MM + DMDF/Encephalomyopathy/ | m.14709T > C | 14709 |
| | | Dementia + diabetes + ophthalmoplegia | | |
| 94 | MT-TE | Encephalomyopathy + Retinopathy | m.14710G > A | 14710 |
| 95 | MT-CYB | EXIT/Septo-Optic Dysplasia | m.14849T > C | 14849 |
| 96 | MT-CYB | Multisystem Disorder, EXIT | m.15579A > G | 15579 |
| 97 | MT-TP | MM/PEO | m.15990C > T | 15990 |

What is claimed is:

1. A nucleobase editor system comprising:
a first unit comprising a first double-stranded (ds) DNA binding polypeptide associated with a single-stranded (ss-) nickase; and
a second unit comprising a second dsDNA binding polypeptide associated with a deaminase,
wherein the nucleobase editor system is configured such that the first dsDNA binding polypeptide of the first unit and the second dsDNA binding polypeptide of the second unit, when associated with a dsDNA, position the ss-nickase and the deaminase such that a site action for the ss-nickase and a site of action for the deaminase are within an editing region on the dsDNA.

2. The nucleobase editor system of claim 1, wherein the ss-nickase of the first unit recognizes a recognition sequence present in the editing region of the dsDNA.

3. The nucleobase editor system of claim 2, wherein the recognition sequence of the ss-nickase is a palindromic recognition sequence.

4. The nucleobase editor system of claim 2 or 3, wherein the recognition sequence of the ss-nickase is 5′-GATC-3′.

5. The nucleobase editor system of claim 2, wherein the recognition sequence of the ss-nickase is a non-palindromic recognition sequence.

6. The nucleobase editor system of claim 5, wherein the recognition sequence of the ss-nickase is 5′-GATD-3′, where D is a base selected from the group consisting of G, A, and T.

7. The nucleobase editor system of claim 5, wherein the recognition sequence of the ss-nickase is 5′-GAGTC-3′.

8. The nucleobase editor system of any one of claims 2-7, wherein the recognition sequence of the ss-nickase is hemimethylated.

9. The nucleobase editor system of any one of claims 1-8, wherein the first dsDNA binding polypeptide of the first unit comprises a transcription activator-like effector (TALE) domain.

10. The nucleobase editor system of any one of claims 1-8, wherein the first dsDNA binding polypeptide of the first unit comprises a zinc finger (ZF) domain.

11. The nucleobase editor system of any one of claims 1-10, wherein the first dsDNA binding polypeptide of the first unit specifically associates with a portion of the dsDNA upstream of the editing region on the dsDNA sequence.

12. The nucleobase editor system of any one of claims 1-10, wherein the first dsDNA binding polypeptide of the first unit specifically associates with a portion of the dsDNA downstream of the editing region on the dsDNA sequence.

13. The nucleobase editor system of any one of claims 2-12, wherein the site of action of the ss-nickase is on the same strand of the dsDNA that is bound by the first dsDNA binding polypeptide of the first unit.

14. The nucleobase editor system of claim 13, wherein the first dsDNA binding polypeptide of the first unit binds the dsDNA 5-9 bases away from the recognition sequence of the ss-nickase.

15. The nucleobase editor system of any one of claims 2-12, wherein the site of action of the ss-nickase is on the opposite strand of the dsDNA that is bound by the first dsDNA binding polypeptide of the first unit.

16. The nucleobase editor system of claim 15, wherein the first dsDNA binding polypeptide of the first unit binds 0-4 bases away from the recognition sequence of the ss-nickase.

17. The nucleobase editor system of any one of claims 1-16, wherein the ss-nickase is heterologous.

18. The nucleobase editor system of any one of claims 1-17, wherein the ss-nickase of the first unit is a type I nickase, type II nickase, type III nickase, or a type IV nickase.

19. The nucleobase editor system of claims 1-18, wherein the ss-nickase is MutH or Nt.BspD6I, or a nickase derived therefrom.

20. The nucleobase editor system of any one of claims 1-19, wherein the second dsDNA binding polypeptide of the second unit comprises a transcription activator-like effector (TALE) domain.

21. The nucleobase editor system of any one of claims 1-19, wherein the second dsDNA binding polypeptide of the second unit comprises a zinc finger (ZF) domain.

22. The nucleobase editor system of any one of claims 1-21, wherein the second dsDNA binding polypeptide binds to the stand of the dsDNA not bound by the first dsDNA binding polypeptide of the first unit.

23. The nucleobase editor system of claim 11, wherein the second dsDNA binding polypeptide of the second unit specifically associates with a portion of the dsDNA downstream of the editing region on the dsDNA sequence.

24. The nucleobase editor system of claim 12, wherein the second dsDNA binding polypeptide of the second unit specifically associates with a portion of the dsDNA upstream of the editing region on the dsDNA sequence.

25. The nucleobase editor system of any one of claims 1-24, wherein the site of action of the deaminase is part of the non-nicked strand of the dsDNA.

26. The nucleobase editor system of any one of claims 1-25, wherein the deaminase is heterologous.

27. The nucleobase editor system of any one of claims 1-26, wherein the deaminase is a single-stranded deaminase (ss-deaminase).

28. The nucleobase editor system of any one of claims 1-27, wherein the deaminase of the second unit is a cytosine-to-uracil deaminase, a 5-methylcytosine-to-thymine deaminase, a guanine-to-xanthine deaminase, an adenine-to-hypoxanthine deaminase, or an adenine-to-inosine deaminase.

29. The nucleobase editor system of claims 1-28, wherein the deaminase is TadA8e, APOBEC, or AID.

30. The nucleobase editor system of any one of claims 1-29, wherein the editing region on the dsDNA is 1-24 base pairs in length.

31. The nucleobase editor system of any one of claims 1-30, wherein the site of action of the ss-nickase is no more than 10 base pairs from the site of action of the deaminase.

32. The nucleobase editor system of any one of claims 1-31, wherein the first unit is a fusion polypeptide, wherein the first dsDNA binding polypeptide of the first unit is fused to the ss-nickase of the first unit.

33. The nucleobase editor system of claim 32, wherein the first dsDNA binding polypeptide of the first unit is fused to the C-terminus of the ss-nickase of the first unit.

34. The nucleobase editor system of claim 32, wherein the first dsDNA binding polypeptide of the first unit is fused to the N-terminus of the ss-nickase of the first unit.

35. The nucleobase editor system of any one of claims 32-34, wherein the first unit further comprises a linker associating the first dsDNA binding polypeptide and the ss-nickase.

36. The nucleobase editor system of any one of claims 1-35, wherein the second unit is a fusion polypeptide, wherein the second dsDNA binding polypeptide of the second unit is fused to the deaminase of the second unit.

37. The nucleobase editor system of claim 36, wherein the second dsDNA binding polypeptide of the second unit is fused to the C-terminus of the deaminase of the second unit.

38. The nucleobase editor system of claim 36, wherein the second dsDNA binding polypeptide of the second unit is fused to the N-terminus of the deaminase of the second unit.

39. The nucleobase editor system of any one of claims 36-38, wherein the second unit further comprises a linker associating the second dsDNA binding polypeptide and the deaminase.

40. The nucleobase editor system of claim 35 or 38, wherein the linker of the first unit and/or the linker of the second unit comprise a polypeptide linker.

41. The nucleobase editor system of claim 40, wherein the polypeptide linker is from 2-100 amino acids in length.

42. The nucleobase editor system of any one of claims 1-31, wherein the first dsDNA binding polypeptide and the ss-nickase of the first unit associate non-covalently.

43. The nucleobase editor system of any one of claims 1-31, wherein the second dsDNA binding polypeptide and the deaminase of the second unit associate non-covalently.

44. The nucleobase editor system of any one of claims 1-43, wherein the first unit further comprises a mitochondrial localization signal (MLS).

45. The nucleobase editor system of claim 44, wherein the MLS is positioned at the N-terminus of the first unit.

46. The nucleobase editor system of any one of claims 1-45, wherein the second unit further comprises a mitochondrial localization signal (MLS).

47. The nucleobase editor system of claim 46, wherein the MLS is positioned at the N-terminus of the second unit.

48. The nucleobase editor system of any one of claims 1-47, wherein the dsDNA is a circularized dsDNA.

49. The nucleobase editor system of any one of claims 1-48, wherein the dsDNA is mitochondrial DNA (mtDNA).

50. The nucleobase editor system of any one of claims 1-49, wherein the dsDNA is a B-DNA conformation.

51. A nucleobase editor system comprising:
a single-stranded (ss-) nickase;
a deaminase; and
a double-stranded (ds) DNA binding polypeptide,
wherein the ss-nickase, the deaminase, and dsDNA binding polypeptide form a complex configured such that the dsDNA binding polypeptide, when associated with a dsDNA, positions the ss-nickase and the deaminase such that a site action for the ss-nickase and a site of action for the deaminase are within an editing region on the dsDNA.

52. A non-naturally occurring polynucleotide encoding:
a first unit comprising a first double-stranded (ds) DNA binding polypeptide associated with a single-stranded (ss-) nickase; and/or
a second unit comprising a second dsDNA binding polypeptide associated with a deaminase.

53. A non-naturally occurring polynucleotide encoding:
a first unit comprising a first double-stranded (ds) DNA binding polypeptide associated with a single-stranded (ss-) nickase; and
a second unit comprising a second dsDNA binding polypeptide associated with a deaminase,
wherein the first unit and second unit, when expressed, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

54. A non-naturally occurring polynucleotide encoding:
a single-stranded (ss-) nickase;
a deaminase; and
a double-stranded (ds) DNA binding polypeptide,
wherein the ss-nickase, the deaminase, and dsDNA binding polypeptide, when expressed, form a complex configured such that the dsDNA binding polypeptide, when associated with a dsDNA, positions the ss-nickase and the deaminase with a site action for the ss-nickase and a site of action for the deaminase within an editing region on the dsDNA.

55. A method of editing a target nucleotide in an editing region in a cell, the method comprising delivering the nucleobase editor system of any one of claims 1-51 or the polynucleotide of any one of claims 52-54 to the cell.

56. The method of claim 55, wherein the editing region is on a mitochondrial DNA.

57. A method of treating an individual having a disease associated with a DNA mutation, the method comprising administering one or more polynucleotides encoding:
a first unit comprising a first double-stranded (ds) DNA binding polypeptide associated with a single-stranded (ss-) nickase; and
a second unit comprising a second dsDNA binding polypeptide associated with a deaminase,
wherein the first unit and second unit, when expressed in the individual, are configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

58. The method of claim 57, wherein the mutated DNA is mitochondrial DNA.

59. A kit for a nucleobase editing system, the kit comprising:
a first unit comprising a first double-stranded (ds) DNA binding polypeptide associated with a single-stranded (ss-) nickase; and
a second unit comprising a second dsDNA binding polypeptide associated with a deaminase,
wherein the nucleobase editor system is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

60. A kit for a nucleobase editor system, the kit comprising one or more polynucleotides encoding:
a first unit comprising a first double-stranded (ds) DNA binding polypeptide associated with a single-stranded (ss-) nickase; and
a second unit comprising a second dsDNA binding polypeptide associated with a deaminase,
wherein the nucleobase editor system, when expressed, is configured such that the first dsDNA binding polypeptide of the first unit and the second dsDNA binding polypeptide of the second unit, when associated with a dsDNA, position the ss-nickase and the deaminase with a site of action for the ss-nickase and a site of action for the deaminase within an editing region on the dsDNA.

61. A non-naturally occurring polypeptide having nickase activity, the non-naturally occurring polypeptide comprising the amino acid sequence of SEQ ID NO:1 with the following mutations of E91A and F94A (SEQ ID NO:2).

62. The non-naturally occurring polypeptide of claim 61, wherein the polypeptide is isolated.

63. A polynucleotide encoding the non-naturally occurring polypeptide of claim 61.
